SEQUENCE LISTING (55 sequences)

SEQ ID NO: 1 - protein, 925 aa, Artificial sequence
  hGAAwt w/o sp
GHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 2 - protein, 952 aa, homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 3 - protein, 18 aa, Artificial sequence
  sp7
MAFLWLLSCWALLGTTFG

SEQ ID NO: 4 - protein, 27 aa, Artificial sequence
  sp1
MGVRHPPCSHRLLAVCALVSLATAALL

SEQ ID NO: 5 - protein, 24 aa, Artificial sequence
  sp2
MPSSVSWGILLLAGLCCLVPVSLA

SEQ ID NO: 6 - protein, 25 aa, Artificial sequence
  sp6
MPPPRTGRGLLWLGLVLSSVCVALG

SEQ ID NO: 7 - protein, 22 aa, Artificial sequence
  sp8
MASRLTLLTLLLLLLAGDRASS

SEQ ID NO: 8 - DNA, 2859 bp, homo sapiens
atgggagtgaggcacccgccctgctcccaccggctcctggccgtctgcgccctcgtgtccttggcaaccgcagcgctcctggggcacatcctactccatgatttcctgctggttccccgagagctgagtggctcctccccagtcctggaggagactcacccagctcaccagcagggagccagcagaccagggccccgggatgcccaggcacaccccgggcggccgcgagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 9 - DNA, 2778 bp, Artificial sequence
  hGAAwt w/o sp
gggcacatcctactccatgatttcctgctggttccccgagagctgagtggctcctccccagtcctggaggagactcacccagctcaccagcagggagccagcagaccagggccccgggatgcccaggcacaccccgggcggccgcgagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 10 - DNA, 2778 bp, Artificial sequence
  hGAAco1 w/o sp
ggccatatcctgctgcacgactttctactagtgcccagagagctgagcggcagctctcccgtgctggaagaaacacaccctgcccatcagcagggcgcctctagacctggacctagagatgcccaggcccaccccggcagacctagagctgtgcctacccagtgtgacgtgccccccaacagcagattcgactgcgcccctgacaaggccatcacccaggaacagtgcgaggccagaggctgctgctacatccctgccaagcagggactgcagggcgctcagatgggacagccctggtgcttcttcccaccctcctaccccagctacaagctggaaaacctgagcagcagcgagatgggctacaccgccaccctgaccagaaccacccccacattcttcccaaaggacatcctgaccctgcggctggacgtgatgatggaaaccgagaaccggctgcacttcaccatcaaggaccccgccaatcggagatacgaggtgcccctggaaaccccccacgtgcactctagagcccccagccctctgtacagcgtggaattcagcgaggaacccttcggcgtgatcgtgcggagacagctggatggcagagtgctgctgaacaccaccgtggcccctctgttcttcgccgaccagttcctgcagctgagcaccagcctgcccagccagtacatcacaggactggccgagcacctgagccccctgatgctgagcacatcctggacccggatcaccctgtggaacagggatctggcccctacccctggcgccaatctgtacggcagccaccctttctacctggccctggaagatggcggatctgcccacggagtgtttctgctgaactccaacgccatggacgtggtgctgcagcctagccctgccctgtcttggagaagcacaggcggcatcctggatgtgtacatctttctgggccccgagcccaagagcgtggtgcagcagtatctggatgtcgtgggctaccccttcatgcccccttactggggcctgggattccacctgtgcagatggggctactccagcaccgccatcaccagacaggtggtggaaaacatgaccagagcccacttcccactggatgtgcagtggaacgacctggactacatggacagcagacgggacttcaccttcaacaaggacggcttccgggacttccccgccatggtgcaggaactgcatcagggcggcagacggtacatgatgatcgtggatcccgccatcagctcctctggccctgccggctcttacagaccctacgacgagggcctgcggagaggcgtgttcatcaccaacgagacaggccagcccctgatcggcaaagtgtggcctggcagcacagccttccccgacttcaccaatcctaccgccctggcttggtgggaggacatggtggccgagttccacgaccaggtgcccttcgacggcatgtggatcgacatgaacgagcccagcaacttcatccggggcagcgaggatggctgccccaacaacgaactggaaaatcccccttacgtgcccggcgtcgtgggcggaacactgcaggccgctacaatctgtgccagcagccaccagtttctgagcacccactacaacctgcacaacctgtacggcctgaccgaggccattgccagccaccgcgctctcgtgaaagccagaggcacacggcccttcgtgatcagcagaagcacctttgccggccacggcagatacgccggacattggactggcgacgtgtggtcctcttgggagcagctggcctctagcgtgcccgagatcctgcagttcaatctgctgggcgtgccactcgtgggcgccgatgtgtgtggcttcctgggcaacacctccgaggaactgtgtgtgcggtggacacagctgggcgccttctaccctttcatgagaaaccacaacagcctgctgagcctgccccaggaaccctacagctttagcgagcctgcacagcaggccatgcggaaggccctgacactgagatacgctctgctgccccacctgtacaccctgtttcaccaggcccatgtggccggcgagacagtggccagacctctgtttctggaattccccaaggacagcagcacctggaccgtggaccatcagctgctgtggggagaggctctgctgattaccccagtgctgcaggcaggcaaggccgaagtgaccggctactttcccctgggcacttggtacgacctgcagaccgtgcctgtggaagccctgggatctctgcctccacctcctgccgctcctagagagcctgccattcactctgagggccagtgggtcacactgcctgcccccctggataccatcaacgtgcacctgagggccggctacatcataccactgcagggacctggcctgaccaccaccgagtctagacagcagccaatggccctggccgtggccctgaccaaaggcggagaagctaggggcgagctgttctgggacgatggcgagagcctggaagtgctggaaagaggcgcctatacccaagtgatcttcctggcccggaacaacaccatcgtgaacgagctggtgcgcgtgacctctgaaggcgctggactgcagctgcagaaagtgaccgtgctgggagtggccacagcccctcagcaggtgctgtctaatggcgtgcccgtgtccaacttcacctacagccccgacaccaaggtgctggacatctgcgtgtcactgctgatgggagagcagtttctggtgtcctggtgctga

SEQ ID NO: 11 - DNA, 2778 bp, Artificial sequence
  hGAAco2 w/o sp
ggacacatcctgctgcacgacttcctgttggtgcctagagagctgagcggatcatccccagtgctggaggagactcatcctgctcaccaacagggagcttccagaccaggaccgagagacgcccaagcccatcctggtagaccaagagctgtgcctacccaatgcgacgtgccacccaactcccgattcgactgcgcgccagataaggctattacccaagagcagtgtgaagccagaggttgctgctacatcccagcgaagcaaggattgcaaggcgcccaaatgggacaaccttggtgtttcttccccccttcgtacccatcatataaactcgaaaacctgtcctcttcggaaatgggttatactgccaccctcaccagaactactcctactttcttcccgaaagacatcttgaccttgaggctggacgtgatgatggagactgaaaaccggctgcatttcactatcaaagatcctgccaatcggcgatacgaggtccctctggaaacccctcacgtgcactcacgggctccttctccgctttactccgtcgaattctctgaggaacccttcggagtgatcgttagacgccagctggatggtagagtgctgttgaacactactgtggccccacttttcttcgctgaccagtttctgcaactgtccacttccctgccatcccagtacattactggactcgccgaacacctgtcgccactgatgctctcgacctcttggactagaatcactttgtggaacagagacttggcccctactccgggagcaaatctgtacggaagccaccctttttacctggcgctcgaagatggcggatccgctcacggagtgttcctgctgaatagcaacgcaatggacgtggtgctgcaaccttcccctgcactcagttggagaagtaccgggggtattctggacgtgtacatcttcctcggaccagaacccaagagcgtggtgcagcaatatctggacgtggtcggatacccttttatgcctccttactggggactgggattccacctttgccgttggggctactcatccaccgccattaccagacaggtggtggagaatatgaccagagcccacttccctctcgacgtgcagtggaacgatctggactatatggactcccggagagatttcaccttcaacaaggacgggttccgcgattttcccgcgatggttcaagagctccaccagggtggtcgaagatatatgatgatcgtcgacccagccatttcgagcagcggacccgctggatcttatagaccttacgacgaaggccttaggagaggagtgttcatcacaaacgagactggacagcctttgatcggtaaagtgtggcctggatcaaccgcctttcctgactttaccaatcccactgccttggcttggtgggaggacatggtggccgaattccacgaccaagtcccctttgatggaatgtggatcgatatgaacgaaccaagcaattttatcagaggttccgaagacggttgccccaacaacgaactggaaaaccctccttatgtgcccggagtcgtgggcggaacattacaggccgcgactatttgcgccagcagccaccaattcctgtccactcactacaacctccacaacctttatggattaaccgaagctattgcaagtcacagggctctggtgaaggctagagggactaggccctttgtgatctcccgatccacctttgccggacacgggagatacgccggtcactggactggtgacgtgtggagctcatgggaacaactggcctcctccgtgccggaaatcttacagttcaaccttctgggtgtccctcttgtcggagcagacgtgtgtgggtttcttggtaacacctccgaggaactgtgtgtgcgctggactcaactgggtgcattctacccattcatgagaaaccacaactccttgctgtccctgccacaagagccctactcgttcagcgagcctgcacaacaggctatgcggaaggcactgaccctgagatacgccctgcttccacacttatacactctcttccatcaagcgcatgtggcaggagaaaccgttgcaaggcctcttttccttgaattccccaaggattcctcgacttggacggtggatcatcagctgctgtggggagaagctctgctgattactccagtgttgcaagccggaaaagctgaggtgaccggatactttccgctgggaacctggtacgacctccagactgtccctgttgaagcccttggatcactgcctccgcctccggcagctccacgcgaaccagctatacattccgagggacagtgggttacattaccagctcctctggacacaatcaacgtccacttaagagctggctacattatccctctgcaaggaccaggactgactacgaccgagagcagacagcagccaatggcactggctgtggctctgaccaagggaggggaagctagaggagaactcttctgggatgatggggagtcccttgaagtgctggaaagaggcgcttacactcaagtcattttccttgcacggaacaacaccattgtgaacgaattggtgcgagtgaccagcgaaggagctggacttcaactgcagaaggtcactgtgctcggagtggctaccgctcctcagcaagtgctgtcgaatggagtccccgtgtcaaactttacctactcccctgacactaaggtgctcgacatttgcgtgtccctcctgatgggagagcagttccttgtgtcctggtgttga

SEQ ID NO: 12 - DNA, 2754 bp, Artificial sequence
  hGAAco1-delta-8 w/o sp
ctactagtgcccagagagctgagcggcagctctcccgtgctggaagaaacacaccctgcccatcagcagggcgcctctagacctggacctagagatgcccaggcccaccccggcagacctagagctgtgcctacccagtgtgacgtgccccccaacagcagattcgactgcgcccctgacaaggccatcacccaggaacagtgcgaggccagaggctgctgctacatccctgccaagcagggactgcagggcgctcagatgggacagccctggtgcttcttcccaccctcctaccccagctacaagctggaaaacctgagcagcagcgagatgggctacaccgccaccctgaccagaaccacccccacattcttcccaaaggacatcctgaccctgcggctggacgtgatgatggaaaccgagaaccggctgcacttcaccatcaaggaccccgccaatcggagatacgaggtgcccctggaaaccccccacgtgcactctagagcccccagccctctgtacagcgtggaattcagcgaggaacccttcggcgtgatcgtgcggagacagctggatggcagagtgctgctgaacaccaccgtggcccctctgttcttcgccgaccagttcctgcagctgagcaccagcctgcccagccagtacatcacaggactggccgagcacctgagccccctgatgctgagcacatcctggacccggatcaccctgtggaacagggatctggcccctacccctggcgccaatctgtacggcagccaccctttctacctggccctggaagatggcggatctgcccacggagtgtttctgctgaactccaacgccatggacgtggtgctgcagcctagccctgccctgtcttggagaagcacaggcggcatcctggatgtgtacatctttctgggccccgagcccaagagcgtggtgcagcagtatctggatgtcgtgggctaccccttcatgcccccttactggggcctgggattccacctgtgcagatggggctactccagcaccgccatcaccagacaggtggtggaaaacatgaccagagcccacttcccactggatgtgcagtggaacgacctggactacatggacagcagacgggacttcaccttcaacaaggacggcttccgggacttccccgccatggtgcaggaactgcatcagggcggcagacggtacatgatgatcgtggatcccgccatcagctcctctggccctgccggctcttacagaccctacgacgagggcctgcggagaggcgtgttcatcaccaacgagacaggccagcccctgatcggcaaagtgtggcctggcagcacagccttccccgacttcaccaatcctaccgccctggcttggtgggaggacatggtggccgagttccacgaccaggtgcccttcgacggcatgtggatcgacatgaacgagcccagcaacttcatccggggcagcgaggatggctgccccaacaacgaactggaaaatcccccttacgtgcccggcgtcgtgggcggaacactgcaggccgctacaatctgtgccagcagccaccagtttctgagcacccactacaacctgcacaacctgtacggcctgaccgaggccattgccagccaccgcgctctcgtgaaagccagaggcacacggcccttcgtgatcagcagaagcacctttgccggccacggcagatacgccggacattggactggcgacgtgtggtcctcttgggagcagctggcctctagcgtgcccgagatcctgcagttcaatctgctgggcgtgccactcgtgggcgccgatgtgtgtggcttcctgggcaacacctccgaggaactgtgtgtgcggtggacacagctgggcgccttctaccctttcatgagaaaccacaacagcctgctgagcctgccccaggaaccctacagctttagcgagcctgcacagcaggccatgcggaaggccctgacactgagatacgctctgctgccccacctgtacaccctgtttcaccaggcccatgtggccggcgagacagtggccagacctctgtttctggaattccccaaggacagcagcacctggaccgtggaccatcagctgctgtggggagaggctctgctgattaccccagtgctgcaggcaggcaaggccgaagtgaccggctactttcccctgggcacttggtacgacctgcagaccgtgcctgtggaagccctgggatctctgcctccacctcctgccgctcctagagagcctgccattcactctgagggccagtgggtcacactgcctgcccccctggataccatcaacgtgcacctgagggccggctacatcataccactgcagggacctggcctgaccaccaccgagtctagacagcagccaatggccctggccgtggccctgaccaaaggcggagaagctaggggcgagctgttctgggacgatggcgagagcctggaagtgctggaaagaggcgcctatacccaagtgatcttcctggcccggaacaacaccatcgtgaacgagctggtgcgcgtgacctctgaaggcgctggactgcagctgcagaaagtgaccgtgctgggagtggccacagcccctcagcaggtgctgtctaatggcgtgcccgtgtccaacttcacctacagccccgacaccaaggtgctggacatctgcgtgtcactgctgatgggagagcagtttctggtgtcctggtgctga

SEQ ID NO: 13 - DNA, 2754 bp, Artificial sequence
  hGAAco2-delta8 w/o sp
ctgttggtgcctagagagctgagcggatcatccccagtgctggaggagactcatcctgctcaccaacagggagcttccagaccaggaccgagagacgcccaagcccatcctggtagaccaagagctgtgcctacccaatgcgacgtgccacccaactcccgattcgactgcgcgccagataaggctattacccaagagcagtgtgaagccagaggttgctgctacatcccagcgaagcaaggattgcaaggcgcccaaatgggacaaccttggtgtttcttccccccttcgtacccatcatataaactcgaaaacctgtcctcttcggaaatgggttatactgccaccctcaccagaactactcctactttcttcccgaaagacatcttgaccttgaggctggacgtgatgatggagactgaaaaccggctgcatttcactatcaaagatcctgccaatcggcgatacgaggtccctctggaaacccctcacgtgcactcacgggctccttctccgctttactccgtcgaattctctgaggaacccttcggagtgatcgttagacgccagctggatggtagagtgctgttgaacactactgtggccccacttttcttcgctgaccagtttctgcaactgtccacttccctgccatcccagtacattactggactcgccgaacacctgtcgccactgatgctctcgacctcttggactagaatcactttgtggaacagagacttggcccctactccgggagcaaatctgtacggaagccaccctttttacctggcgctcgaagatggcggatccgctcacggagtgttcctgctgaatagcaacgcaatggacgtggtgctgcaaccttcccctgcactcagttggagaagtaccgggggtattctggacgtgtacatcttcctcggaccagaacccaagagcgtggtgcagcaatatctggacgtggtcggatacccttttatgcctccttactggggactgggattccacctttgccgttggggctactcatccaccgccattaccagacaggtggtggagaatatgaccagagcccacttccctctcgacgtgcagtggaacgatctggactatatggactcccggagagatttcaccttcaacaaggacgggttccgcgattttcccgcgatggttcaagagctccaccagggtggtcgaagatatatgatgatcgtcgacccagccatttcgagcagcggacccgctggatcttatagaccttacgacgaaggccttaggagaggagtgttcatcacaaacgagactggacagcctttgatcggtaaagtgtggcctggatcaaccgcctttcctgactttaccaatcccactgccttggcttggtgggaggacatggtggccgaattccacgaccaagtcccctttgatggaatgtggatcgatatgaacgaaccaagcaattttatcagaggttccgaagacggttgccccaacaacgaactggaaaaccctccttatgtgcccggagtcgtgggcggaacattacaggccgcgactatttgcgccagcagccaccaattcctgtccactcactacaacctccacaacctttatggattaaccgaagctattgcaagtcacagggctctggtgaaggctagagggactaggccctttgtgatctcccgatccacctttgccggacacgggagatacgccggtcactggactggtgacgtgtggagctcatgggaacaactggcctcctccgtgccggaaatcttacagttcaaccttctgggtgtccctcttgtcggagcagacgtgtgtgggtttcttggtaacacctccgaggaactgtgtgtgcgctggactcaactgggtgcattctacccattcatgagaaaccacaactccttgctgtccctgccacaagagccctactcgttcagcgagcctgcacaacaggctatgcggaaggcactgaccctgagatacgccctgcttccacacttatacactctcttccatcaagcgcatgtggcaggagaaaccgttgcaaggcctcttttccttgaattccccaaggattcctcgacttggacggtggatcatcagctgctgtggggagaagctctgctgattactccagtgttgcaagccggaaaagctgaggtgaccggatactttccgctgggaacctggtacgacctccagactgtccctgttgaagcccttggatcactgcctccgcctccggcagctccacgcgaaccagctatacattccgagggacagtgggttacattaccagctcctctggacacaatcaacgtccacttaagagctggctacattatccctctgcaaggaccaggactgactacgaccgagagcagacagcagccaatggcactggctgtggctctgaccaagggaggggaagctagaggagaactcttctgggatgatggggagtcccttgaagtgctggaaagaggcgcttacactcaagtcattttccttgcacggaacaacaccattgtgaacgaattggtgcgagtgaccagcgaaggagctggacttcaactgcagaaggtcactgtgctcggagtggctaccgctcctcagcaagtgctgtcgaatggagtccccgtgtcaaactttacctactcccctgacactaaggtgctcgacatttgcgtgtccctcctgatgggagagcagttccttgtgtcctggtgttga

SEQ ID NO: 14 - DNA, 397 bp, Artificial sequence
  hAAT promoter
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 15 - DNA, 321 bp, Artificial sequence
  ApoE control region
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 16 - DNA, 441 bp, Artificial sequence
  HBB2 intron
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaag

SEQ ID NO: 17 - DNA, 441 bp, Artificial sequence
  modified HBB2 intron
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaag

SEQ ID NO: 18 - DNA, 1438 bp, Artificial sequence
  FIX intron
ggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacaatgcttatgatgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaaatgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaacgcttttcatggattaggaaaaaatcattttgtctctatgtcaaacatcttggagttgatatttggggaaacacaatactcagttgagttccctaggggagaaaagcacgcttaagaattgacataaagagtaggaagttagctaatgcaacatatatcactttgttttttcacaactacagtgactttatgtatttcccagaggaaggcatacagggaagaaattatcccatttggacaaacagcatgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttca

SEQ ID NO: 19 - DNA, 1438 bp, Artificial sequence
  modified FIX intron
ggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacattgcttttgttgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaattgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaacgcttttcatggattaggaaaaaatcattttgtctctttgtcaaacatcttggagttgatatttggggaaacacaatactcagttgagttccctaggggagaaaagcacgcttaagaattgacataaagagtaggaagttagctattgcaacatatatcactttgttttttcacaactacagtgactttttgtatttcccagaggaaggcatacagggaagaaattatcccatttggacaaacagcttgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgttggatcactttgcaaagatcctcattgagctattttcaagtgttgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcttgttgttttcttttttgctaaaactaaagaattattcttttacatttca

SEQ ID NO: 20 - DNA, 881 bp, Artificial sequence
  chicken beta-globin intron
gcgggagtcgctgcgttgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctc

SEQ ID NO: 21 - DNA, 881 bp, Artificial sequence
  modified chicken beta-globin intron
gcgggagtcgctgcgttgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttttggtaatcgtgcgagagggcgcagggacttcctttgtcccaaatctgtgcggagccgaaatctgggaggcgccgccgcaccccctctagcgggcgcggggcgaagcggtgcggcgccggcaggaaggaattgggcggggagggccttcgtgcgtcgccgcgccgccgtccccttctccctc

SEQ ID NO: 22 - DNA, 4318 bp, Artificial sequence
  construct sp2+hGAAwt-delta-8
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatgccgtcttctgtctcgtggggcatcctcctgctggcaggcctgtgctgcctggtccctgtctccctggctctgctggttccccgagagctgagtggctcctccccagtcctggaggagactcacccagctcaccagcagggagccagcagaccagggccccgggatgcccaggcacaccccgggcggccgcgagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagctcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 23 - DNA, 4318 bp, Artificial sequence
  construct sp2+hGAAco1-delta-8
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatgcctagctctgtgtcctggggcattctgctgctggccggcctgtgttgtctggtgcctgtgtctctggccctactagtgcccagagagctgagcggcagctctcccgtgctggaagaaacacaccctgcccatcagcagggcgcctctagacctggacctagagatgcccaggcccaccccggcagacctagagctgtgcctacccagtgtgacgtgccccccaacagcagattcgactgcgcccctgacaaggccatcacccaggaacagtgcgaggccagaggctgctgctacatccctgccaagcagggactgcagggcgctcagatgggacagccctggtgcttcttcccaccctcctaccccagctacaagctggaaaacctgagcagcagcgagatgggctacaccgccaccctgaccagaaccacccccacattcttcccaaaggacatcctgaccctgcggctggacgtgatgatggaaaccgagaaccggctgcacttcaccatcaaggaccccgccaatcggagatacgaggtgcccctggaaaccccccacgtgcactctagagcccccagccctctgtacagcgtggaattcagcgaggaacccttcggcgtgatcgtgcggagacagctggatggcagagtgctgctgaacaccaccgtggcccctctgttcttcgccgaccagttcctgcagctgagcaccagcctgcccagccagtacatcacaggactggccgagcacctgagccccctgatgctgagcacatcctggacccggatcaccctgtggaacagggatctggcccctacccctggcgccaatctgtacggcagccaccctttctacctggccctggaagatggcggatctgcccacggagtgtttctgctgaactccaacgccatggacgtggtgctgcagcctagccctgccctgtcttggagaagcacaggcggcatcctggatgtgtacatctttctgggccccgagcccaagagcgtggtgcagcagtatctggatgtcgtgggctaccccttcatgcccccttactggggcctgggattccacctgtgcagatggggctactccagcaccgccatcaccagacaggtggtggaaaacatgaccagagcccacttcccactggatgtgcagtggaacgacctggactacatggacagcagacgggacttcaccttcaacaaggacggcttccgggacttccccgccatggtgcaggaactgcatcagggcggcagacggtacatgatgatcgtggatcccgccatcagctcctctggccctgccggctcttacagaccctacgacgagggcctgcggagaggcgtgttcatcaccaacgagacaggccagcccctgatcggcaaagtgtggcctggcagcacagccttccccgacttcaccaatcctaccgccctggcttggtgggaggacatggtggccgagttccacgaccaggtgcccttcgacggcatgtggatcgacatgaacgagcccagcaacttcatccggggcagcgaggatggctgccccaacaacgaactggaaaatcccccttacgtgcccggcgtcgtgggcggaacactgcaggccgctacaatctgtgccagcagccaccagtttctgagcacccactacaacctgcacaacctgtacggcctgaccgaggccattgccagccaccgcgctctcgtgaaagccagaggcacacggcccttcgtgatcagcagaagcacctttgccggccacggcagatacgccggacattggactggcgacgtgtggtcctcttgggagcagctggcctctagcgtgcccgagatcctgcagttcaatctgctgggcgtgccactcgtgggcgccgatgtgtgtggcttcctgggcaacacctccgaggaactgtgtgtgcggtggacacagctgggcgccttctaccctttcatgagaaaccacaacagcctgctgagcctgccccaggaaccctacagctttagcgagcctgcacagcaggccatgcggaaggccctgacactgagatacgctctgctgccccacctgtacaccctgtttcaccaggcccatgtggccggcgagacagtggccagacctctgtttctggaattccccaaggacagcagcacctggaccgtggaccatcagctgctgtggggagaggctctgctgattaccccagtgctgcaggcaggcaaggccgaagtgaccggctactttcccctgggcacttggtacgacctgcagaccgtgcctgtggaagccctgggatctctgcctccacctcctgccgctcctagagagcctgccattcactctgagggccagtgggtcacactgcctgcccccctggataccatcaacgtgcacctgagggccggctacatcataccactgcagggacctggcctgaccaccaccgagtctagacagcagccaatggccctggccgtggccctgaccaaaggcggagaagctaggggcgagctgttctgggacgatggcgagagcctggaagtgctggaaagaggcgcctatacccaagtgatcttcctggcccggaacaacaccatcgtgaacgagctggtgcgcgtgacctctgaaggcgctggactgcagctgcagaaagtgaccgtgctgggagtggccacagcccctcagcaggtgctgtctaatggcgtgcccgtgtccaacttcacctacagccccgacaccaaggtgctggacatctgcgtgtcactgctgatgggagagcagtttctggtgtcctggtgctgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 24 - DNA, 4318 bp, Artificial sequence
  construct sp2+hGAAco2-delta-8
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatgccatcgtcagtgtcttggggcattcttctgctcgccggattgtgttgcctggtgcctgtctcattggccctgttggtgcctagagagctgagcggatcatccccagtgctggaggagactcatcctgctcaccaacagggagcttccagaccaggaccgagagacgcccaagcccatcctggtagaccaagagctgtgcctacccaatgcgacgtgccacccaactcccgattcgactgcgcgccagataaggctattacccaagagcagtgtgaagccagaggttgctgctacatcccagcgaagcaaggattgcaaggcgcccaaatgggacaaccttggtgtttcttccccccttcgtacccatcatataaactcgaaaacctgtcctcttcggaaatgggttatactgccaccctcaccagaactactcctactttcttcccgaaagacatcttgaccttgaggctggacgtgatgatggagactgaaaaccggctgcatttcactatcaaagatcctgccaatcggcgatacgaggtccctctggaaacccctcacgtgcactcacgggctccttctccgctttactccgtcgaattctctgaggaacccttcggagtgatcgttagacgccagctggatggtagagtgctgttgaacactactgtggccccacttttcttcgctgaccagtttctgcaactgtccacttccctgccatcccagtacattactggactcgccgaacacctgtcgccactgatgctctcgacctcttggactagaatcactttgtggaacagagacttggcccctactccgggagcaaatctgtacggaagccaccctttttacctggcgctcgaagatggcggatccgctcacggagtgttcctgctgaatagcaacgcaatggacgtggtgctgcaaccttcccctgcactcagttggagaagtaccgggggtattctggacgtgtacatcttcctcggaccagaacccaagagcgtggtgcagcaatatctggacgtggtcggatacccttttatgcctccttactggggactgggattccacctttgccgttggggctactcatccaccgccattaccagacaggtggtggagaatatgaccagagcccacttccctctcgacgtgcagtggaacgatctggactatatggactcccggagagatttcaccttcaacaaggacgggttccgcgattttcccgcgatggttcaagagctccaccagggtggtcgaagatatatgatgatcgtcgacccagccatttcgagcagcggacccgctggatcttatagaccttacgacgaaggccttaggagaggagtgttcatcacaaacgagactggacagcctttgatcggtaaagtgtggcctggatcaaccgcctttcctgactttaccaatcccactgccttggcttggtgggaggacatggtggccgaattccacgaccaagtcccctttgatggaatgtggatcgatatgaacgaaccaagcaattttatcagaggttccgaagacggttgccccaacaacgaactggaaaaccctccttatgtgcccggagtcgtgggcggaacattacaggccgcgactatttgcgccagcagccaccaattcctgtccactcactacaacctccacaacctttatggattaaccgaagctattgcaagtcacagggctctggtgaaggctagagggactaggccctttgtgatctcccgatccacctttgccggacacgggagatacgccggtcactggactggtgacgtgtggagctcatgggaacaactggcctcctccgtgccggaaatcttacagttcaaccttctgggtgtccctcttgtcggagcagacgtgtgtgggtttcttggtaacacctccgaggaactgtgtgtgcgctggactcaactgggtgcattctacccattcatgagaaaccacaactccttgctgtccctgccacaagagccctactcgttcagcgagcctgcacaacaggctatgcggaaggcactgaccctgagatacgccctgcttccacacttatacactctcttccatcaagcgcatgtggcaggagaaaccgttgcaaggcctcttttccttgaattccccaaggattcctcgacttggacggtggatcatcagctgctgtggggagaagctctgctgattactccagtgttgcaagccggaaaagctgaggtgaccggatactttccgctgggaacctggtacgacctccagactgtccctgttgaagcccttggatcactgcctccgcctccggcagctccacgcgaaccagctatacattccgagggacagtgggttacattaccagctcctctggacacaatcaacgtccacttaagagctggctacattatccctctgcaaggaccaggactgactacgaccgagagcagacagcagccaatggcactggctgtggctctgaccaagggaggggaagctagaggagaactcttctgggatgatggggagtcccttgaagtgctggaaagaggcgcttacactcaagtcattttccttgcacggaacaacaccattgtgaacgaattggtgcgagtgaccagcgaaggagctggacttcaactgcagaaggtcactgtgctcggagtggctaccgctcctcagcaagtgctgtcgaatggagtccccgtgtcaaactttacctactcccctgacactaaggtgctcgacatttgcgtgtccctcctgatgggagagcagttccttgtgtcctggtgttgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 25 - DNA, 4300 bp, Artificial sequence
  construct sp7+hGAAco1-delta-8
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggcctttctgtggctgctgagctgttgggccctgctgggcaccaccttcggcctactagtgcccagagagctgagcggcagctctcccgtgctggaagaaacacaccctgcccatcagcagggcgcctctagacctggacctagagatgcccaggcccaccccggcagacctagagctgtgcctacccagtgtgacgtgccccccaacagcagattcgactgcgcccctgacaaggccatcacccaggaacagtgcgaggccagaggctgctgctacatccctgccaagcagggactgcagggcgctcagatgggacagccctggtgcttcttcccaccctcctaccccagctacaagctggaaaacctgagcagcagcgagatgggctacaccgccaccctgaccagaaccacccccacattcttcccaaaggacatcctgaccctgcggctggacgtgatgatggaaaccgagaaccggctgcacttcaccatcaaggaccccgccaatcggagatacgaggtgcccctggaaaccccccacgtgcactctagagcccccagccctctgtacagcgtggaattcagcgaggaacccttcggcgtgatcgtgcggagacagctggatggcagagtgctgctgaacaccaccgtggcccctctgttcttcgccgaccagttcctgcagctgagcaccagcctgcccagccagtacatcacaggactggccgagcacctgagccccctgatgctgagcacatcctggacccggatcaccctgtggaacagggatctggcccctacccctggcgccaatctgtacggcagccaccctttctacctggccctggaagatggcggatctgcccacggagtgtttctgctgaactccaacgccatggacgtggtgctgcagcctagccctgccctgtcttggagaagcacaggcggcatcctggatgtgtacatctttctgggccccgagcccaagagcgtggtgcagcagtatctggatgtcgtgggctaccccttcatgcccccttactggggcctgggattccacctgtgcagatggggctactccagcaccgccatcaccagacaggtggtggaaaacatgaccagagcccacttcccactggatgtgcagtggaacgacctggactacatggacagcagacgggacttcaccttcaacaaggacggcttccgggacttccccgccatggtgcaggaactgcatcagggcggcagacggtacatgatgatcgtggatcccgccatcagctcctctggccctgccggctcttacagaccctacgacgagggcctgcggagaggcgtgttcatcaccaacgagacaggccagcccctgatcggcaaagtgtggcctggcagcacagccttccccgacttcaccaatcctaccgccctggcttggtgggaggacatggtggccgagttccacgaccaggtgcccttcgacggcatgtggatcgacatgaacgagcccagcaacttcatccggggcagcgaggatggctgccccaacaacgaactggaaaatcccccttacgtgcccggcgtcgtgggcggaacactgcaggccgctacaatctgtgccagcagccaccagtttctgagcacccactacaacctgcacaacctgtacggcctgaccgaggccattgccagccaccgcgctctcgtgaaagccagaggcacacggcccttcgtgatcagcagaagcacctttgccggccacggcagatacgccggacattggactggcgacgtgtggtcctcttgggagcagctggcctctagcgtgcccgagatcctgcagttcaatctgctgggcgtgccactcgtgggcgccgatgtgtgtggcttcctgggcaacacctccgaggaactgtgtgtgcggtggacacagctgggcgccttctaccctttcatgagaaaccacaacagcctgctgagcctgccccaggaaccctacagctttagcgagcctgcacagcaggccatgcggaaggccctgacactgagatacgctctgctgccccacctgtacaccctgtttcaccaggcccatgtggccggcgagacagtggccagacctctgtttctggaattccccaaggacagcagcacctggaccgtggaccatcagctgctgtggggagaggctctgctgattaccccagtgctgcaggcaggcaaggccgaagtgaccggctactttcccctgggcacttggtacgacctgcagaccgtgcctgtggaagccctgggatctctgcctccacctcctgccgctcctagagagcctgccattcactctgagggccagtgggtcacactgcctgcccccctggataccatcaacgtgcacctgagggccggctacatcataccactgcagggacctggcctgaccaccaccgagtctagacagcagccaatggccctggccgtggccctgaccaaaggcggagaagctaggggcgagctgttctgggacgatggcgagagcctggaagtgctggaaagaggcgcctatacccaagtgatcttcctggcccggaacaacaccatcgtgaacgagctggtgcgcgtgacctctgaaggcgctggactgcagctgcagaaagtgaccgtgctgggagtggccacagcccctcagcaggtgctgtctaatggcgtgcccgtgtccaacttcacctacagccccgacaccaaggtgctggacatctgcgtgtcactgctgatgggagagcagtttctggtgtcctggtgctgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 26 - DNA, 4198 bp, Artificial sequence
  sp7+hGAAco1-delta-42
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggcctttctgtggctgctgagctgttgggccctgctgggcaccaccttcggcgcccaccccggcagacctagagctgtgcctacccagtgtgacgtgccccccaacagcagattcgactgcgcccctgacaaggccatcacccaggaacagtgcgaggccagaggctgctgctacatccctgccaagcagggactgcagggcgctcagatgggacagccctggtgcttcttcccaccctcctaccccagctacaagctggaaaacctgagcagcagcgagatgggctacaccgccaccctgaccagaaccacccccacattcttcccaaaggacatcctgaccctgcggctggacgtgatgatggaaaccgagaaccggctgcacttcaccatcaaggaccccgccaatcggagatacgaggtgcccctggaaaccccccacgtgcactctagagcccccagccctctgtacagcgtggaattcagcgaggaacccttcggcgtgatcgtgcggagacagctggatggcagagtgctgctgaacaccaccgtggcccctctgttcttcgccgaccagttcctgcagctgagcaccagcctgcccagccagtacatcacaggactggccgagcacctgagccccctgatgctgagcacatcctggacccggatcaccctgtggaacagggatctggcccctacccctggcgccaatctgtacggcagccaccctttctacctggccctggaagatggcggatctgcccacggagtgtttctgctgaactccaacgccatggacgtggtgctgcagcctagccctgccctgtcttggagaagcacaggcggcatcctggatgtgtacatctttctgggccccgagcccaagagcgtggtgcagcagtatctggatgtcgtgggctaccccttcatgcccccttactggggcctgggattccacctgtgcagatggggctactccagcaccgccatcaccagacaggtggtggaaaacatgaccagagcccacttcccactggatgtgcagtggaacgacctggactacatggacagcagacgggacttcaccttcaacaaggacggcttccgggacttccccgccatggtgcaggaactgcatcagggcggcagacggtacatgatgatcgtggatcccgccatcagctcctctggccctgccggctcttacagaccctacgacgagggcctgcggagaggcgtgttcatcaccaacgagacaggccagcccctgatcggcaaagtgtggcctggcagcacagccttccccgacttcaccaatcctaccgccctggcttggtgggaggacatggtggccgagttccacgaccaggtgcccttcgacggcatgtggatcgacatgaacgagcccagcaacttcatccggggcagcgaggatggctgccccaacaacgaactggaaaatcccccttacgtgcccggcgtcgtgggcggaacactgcaggccgctacaatctgtgccagcagccaccagtttctgagcacccactacaacctgcacaacctgtacggcctgaccgaggccattgccagccaccgcgctctcgtgaaagccagaggcacacggcccttcgtgatcagcagaagcacctttgccggccacggcagatacgccggacattggactggcgacgtgtggtcctcttgggagcagctggcctctagcgtgcccgagatcctgcagttcaatctgctgggcgtgccactcgtgggcgccgatgtgtgtggcttcctgggcaacacctccgaggaactgtgtgtgcggtggacacagctgggcgccttctaccctttcatgagaaaccacaacagcctgctgagcctgccccaggaaccctacagctttagcgagcctgcacagcaggccatgcggaaggccctgacactgagatacgctctgctgccccacctgtacaccctgtttcaccaggcccatgtggccggcgagacagtggccagacctctgtttctggaattccccaaggacagcagcacctggaccgtggaccatcagctgctgtggggagaggctctgctgattaccccagtgctgcaggcaggcaaggccgaagtgaccggctactttcccctgggcacttggtacgacctgcagaccgtgcctgtggaagccctgggatctctgcctccacctcctgccgctcctagagagcctgccattcactctgagggccagtgggtcacactgcctgcccccctggataccatcaacgtgcacctgagggccggctacatcataccactgcagggacctggcctgaccaccaccgagtctagacagcagccaatggccctggccgtggccctgaccaaaggcggagaagctaggggcgagctgttctgggacgatggcgagagcctggaagtgctggaaagaggcgcctatacccaagtgatcttcctggcccggaacaacaccatcgtgaacgagctggtgcgcgtgacctctgaaggcgctggactgcagctgcagaaagtgaccgtgctgggagtggccacagcccctcagcaggtgctgtctaatggcgtgcccgtgtccaacttcacctacagccccgacaccaaggtgctggacatctgcgtgtcactgctgatgggagagcagtttctggtgtcctggtgctgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 27 - protein, 917 aa, Artificial sequence
  hGAA-delta-8
LLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 28 - protein, 883 aa, Artificial sequence
  hGAA-delta-42
AHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 29 - protein, 952 aa, homo sapiens [sequence omitted]

SEQ ID NO: 30 - protein, 952 aa, homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 31 - protein, 957 aa, homo sapiens [sequence omitted]

SEQ ID NO: 32 - protein, 952 aa, homo sapiens [sequence omitted]

SEQ ID NO: 33 - protein, 925 aa, Artificial sequence
  variant hGAAwt w/o sp
GHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 34 - protein, 896 aa, Artificial sequence
  hGAA-delta-29
QQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 35 - protein, 882 aa, Artificial sequence
  hGAA-delta-43
HPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 36 - protein, 878 aa, Artificial sequence
  hGAA-delta-47
PRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 37 - DNA, 2745 bp, Artificial sequence
  sp7+hGAAwt-delta-29
atggcctttctgtggctgctgagctgttgggccctgctgggcaccaccttcggccagcagggagccagcagaccagggccccgggatgcccaggcacaccccgggcggccgcgagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 38 - DNA, 2745 bp, Artificial sequence
  sp7+hGAAco1-delta-29
atggcctttctgtggctgctgagctgttgggccctgctgggcaccaccttcggccagcagggcgcctctagacctggacctagagatgcccaggcccaccccggcagacctagagctgtgcctacccagtgtgacgtgccccccaacagcagattcgactgcgcccctgacaaggccatcacccaggaacagtgcgaggccagaggctgctgctacatccctgccaagcagggactgcagggcgctcagatgggacagccctggtgcttcttcccaccctcctaccccagctacaagctggaaaacctgagcagcagcgagatgggctacaccgccaccctgaccagaaccacccccacattcttcccaaaggacatcctgaccctgcggctggacgtgatgatggaaaccgagaaccggctgcacttcaccatcaaggaccccgccaatcggagatacgaggtgcccctggaaaccccccacgtgcactctagagcccccagccctctgtacagcgtggaattcagcgaggaacccttcggcgtgatcgtgcggagacagctggatggcagagtgctgctgaacaccaccgtggcccctctgttcttcgccgaccagttcctgcagctgagcaccagcctgcccagccagtacatcacaggactggccgagcacctgagccccctgatgctgagcacatcctggacccggatcaccctgtggaacagggatctggcccctacccctggcgccaatctgtacggcagccaccctttctacctggccctggaagatggcggatctgcccacggagtgtttctgctgaactccaacgccatggacgtggtgctgcagcctagccctgccctgtcttggagaagcacaggcggcatcctggatgtgtacatctttctgggccccgagcccaagagcgtggtgcagcagtatctggatgtcgtgggctaccccttcatgcccccttactggggcctgggattccacctgtgcagatggggctactccagcaccgccatcaccagacaggtggtggaaaacatgaccagagcccacttcccactggatgtgcagtggaacgacctggactacatggacagcagacgggacttcaccttcaacaaggacggcttccgggacttccccgccatggtgcaggaactgcatcagggcggcagacggtacatgatgatcgtggatcccgccatcagctcctctggccctgccggctcttacagaccctacgacgagggcctgcggagaggcgtgttcatcaccaacgagacaggccagcccctgatcggcaaagtgtggcctggcagcacagccttccccgacttcaccaatcctaccgccctggcttggtgggaggacatggtggccgagttccacgaccaggtgcccttcgacggcatgtggatcgacatgaacgagcccagcaacttcatccggggcagcgaggatggctgccccaacaacgaactggaaaatcccccttacgtgcccggcgtcgtgggcggaacactgcaggccgctacaatctgtgccagcagccaccagtttctgagcacccactacaacctgcacaacctgtacggcctgaccgaggccattgccagccaccgcgctctcgtgaaagccagaggcacacggcccttcgtgatcagcagaagcacctttgccggccacggcagatacgccggacattggactggcgacgtgtggtcctcttgggagcagctggcctctagcgtgcccgagatcctgcagttcaatctgctgggcgtgccactcgtgggcgccgatgtgtgtggcttcctgggcaacacctccgaggaactgtgtgtgcggtggacacagctgggcgccttctaccctttcatgagaaaccacaacagcctgctgagcctgccccaggaaccctacagctttagcgagcctgcacagcaggccatgcggaaggccctgacactgagatacgctctgctgccccacctgtacaccctgtttcaccaggcccatgtggccggcgagacagtggccagacctctgtttctggaattccccaaggacagcagcacctggaccgtggaccatcagctgctgtggggagaggctctgctgattaccccagtgctgcaggcaggcaaggccgaagtgaccggctactttcccctgggcacttggtacgacctgcagaccgtgcctgtggaagccctgggatctctgcctccacctcctgccgctcctagagagcctgccattcactctgagggccagtgggtcacactgcctgcccccctggataccatcaacgtgcacctgagggccggctacatcataccactgcagggacctggcctgaccaccaccgagtctagacagcagccaatggccctggccgtggccctgaccaaaggcggagaagctaggggcgagctgttctgggacgatggcgagagcctggaagtgctggaaagaggcgcctatacccaagtgatcttcctggcccggaacaacaccatcgtgaacgagctggtgcgcgtgacctctgaaggcgctggactgcagctgcagaaagtgaccgtgctgggagtggccacagcccctcagcaggtgctgtctaatggcgtgcccgtgtccaacttcacctacagccccgacaccaaggtgctggacatctgcgtgtcactgctgatgggagagcagtttctggtgtcctggtgctga

SEQ ID NO: 39 - DNA, 2745 bp, Artificial sequence
  sp7+hGAAco2-delta-29
atggcctttctgtggctgctgagctgttgggccctgctgggcaccaccttcggccaacagggagcttccagaccaggaccgagagacgcccaagcccatcctggtagaccaagagctgtgcctacccaatgcgacgtgccacccaactcccgattcgactgcgcgccagataaggctattacccaagagcagtgtgaagccagaggttgctgctacatcccagcgaagcaaggattgcaaggcgcccaaatgggacaaccttggtgtttcttccccccttcgtacccatcatataaactcgaaaacctgtcctcttcggaaatgggttatactgccaccctcaccagaactactcctactttcttcccgaaagacatcttgaccttgaggctggacgtgatgatggagactgaaaaccggctgcatttcactatcaaagatcctgccaatcggcgatacgaggtccctctggaaacccctcacgtgcactcacgggctccttctccgctttactccgtcgaattctctgaggaacccttcggagtgatcgttagacgccagctggatggtagagtgctgttgaacactactgtggccccacttttcttcgctgaccagtttctgcaactgtccacttccctgccatcccagtacattactggactcgccgaacacctgtcgccactgatgctctcgacctcttggactagaatcactttgtggaacagagacttggcccctactccgggagcaaatctgtacggaagccaccctttttacctggcgctcgaagatggcggatccgctcacggagtgttcctgctgaatagcaacgcaatggacgtggtgctgcaaccttcccctgcactcagttggagaagtaccgggggtattctggacgtgtacatcttcctcggaccagaacccaagagcgtggtgcagcaatatctggacgtggtcggatacccttttatgcctccttactggggactgggattccacctttgccgttggggctactcatccaccgccattaccagacaggtggtggagaatatgaccagagcccacttccctctcgacgtgcagtggaacgatctggactatatggactcccggagagatttcaccttcaacaaggacgggttccgcgattttcccgcgatggttcaagagctccaccagggtggtcgaagatatatgatgatcgtcgacccagccatttcgagcagcggacccgctggatcttatagaccttacgacgaaggccttaggagaggagtgttcatcacaaacgagactggacagcctttgatcggtaaagtgtggcctggatcaaccgcctttcctgactttaccaatcccactgccttggcttggtgggaggacatggtggccgaattccacgaccaagtcccctttgatggaatgtggatcgatatgaacgaaccaagcaattttatcagaggttccgaagacggttgccccaacaacgaactggaaaaccctccttatgtgcccggagtcgtgggcggaacattacaggccgcgactatttgcgccagcagccaccaattcctgtccactcactacaacctccacaacctttatggattaaccgaagctattgcaagtcacagggctctggtgaaggctagagggactaggccctttgtgatctcccgatccacctttgccggacacgggagatacgccggtcactggactggtgacgtgtggagctcatgggaacaactggcctcctccgtgccggaaatcttacagttcaaccttctgggtgtccctcttgtcggagcagacgtgtgtgggtttcttggtaacacctccgaggaactgtgtgtgcgctggactcaactgggtgcattctacccattcatgagaaaccacaactccttgctgtccctgccacaagagccctactcgttcagcgagcctgcacaacaggctatgcggaaggcactgaccctgagatacgccctgcttccacacttatacactctcttccatcaagcgcatgtggcaggagaaaccgttgcaaggcctcttttccttgaattccccaaggattcctcgacttggacggtggatcatcagctgctgtggggagaagctctgctgattactccagtgttgcaagccggaaaagctgaggtgaccggatactttccgctgggaacctggtacgacctccagactgtccctgttgaagcccttggatcactgcctccgcctccggcagctccacgcgaaccagctatacattccgagggacagtgggttacattaccagctcctctggacacaatcaacgtccacttaagagctggctacattatccctctgcaaggaccaggactgactacgaccgagagcagacagcagccaatggcactggctgtggctctgaccaagggaggggaagctagaggagaactcttctgggatgatggggagtcccttgaagtgctggaaagaggcgcttacactcaagtcattttccttgcacggaacaacaccattgtgaacgaattggtgcgagtgaccagcgaaggagctggacttcaactgcagaaggtcactgtgctcggagtggctaccgctcctcagcaagtgctgtcgaatggagtccccgtgtcaaactttacctactcccctgacactaaggtgctcgacatttgcgtgtccctcctgatgggagagcagttccttgtgtcctggtgttga

SEQ ID NO: 40 - DNA, 2706 bp, Artificial sequence
  sp7+hGAAwt-delta-42
atggcctttctgtggctgctgagctgttgggccctgctgggcaccaccttcggcgcacaccccgggcggccgcgagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 41 - DNA, 2706 bp, Artificial sequence
  sp7-hGAAco2-delta-42
atggcctttctgtggctgctgagctgttgggccctgctgggcaccaccttcggcgcccatcctggtagaccaagagctgtgcctacccaatgcgacgtgccacccaactcccgattcgactgcgcgccagataaggctattacccaagagcagtgtgaagccagaggttgctgctacatcccagcgaagcaaggattgcaaggcgcccaaatgggacaaccttggtgtttcttccccccttcgtacccatcatataaactcgaaaacctgtcctcttcggaaatgggttatactgccaccctcaccagaactactcctactttcttcccgaaagacatcttgaccttgaggctggacgtgatgatggagactgaaaaccggctgcatttcactatcaaagatcctgccaatcggcgatacgaggtccctctggaaacccctcacgtgcactcacgggctccttctccgctttactccgtcgaattctctgaggaacccttcggagtgatcgttagacgccagctggatggtagagtgctgttgaacactactgtggccccacttttcttcgctgaccagtttctgcaactgtccacttccctgccatcccagtacattactggactcgccgaacacctgtcgccactgatgctctcgacctcttggactagaatcactttgtggaacagagacttggcccctactccgggagcaaatctgtacggaagccaccctttttacctggcgctcgaagatggcggatccgctcacggagtgttcctgctgaatagcaacgcaatggacgtggtgctgcaaccttcccctgcactcagttggagaagtaccgggggtattctggacgtgtacatcttcctcggaccagaacccaagagcgtggtgcagcaatatctggacgtggtcggatacccttttatgcctccttactggggactgggattccacctttgccgttggggctactcatccaccgccattaccagacaggtggtggagaatatgaccagagcccacttccctctcgacgtgcagtggaacgatctggactatatggactcccggagagatttcaccttcaacaaggacgggttccgcgattttcccgcgatggttcaagagctccaccagggtggtcgaagatatatgatgatcgtcgacccagccatttcgagcagcggacccgctggatcttatagaccttacgacgaaggccttaggagaggagtgttcatcacaaacgagactggacagcctttgatcggtaaagtgtggcctggatcaaccgcctttcctgactttaccaatcccactgccttggcttggtgggaggacatggtggccgaattccacgaccaagtcccctttgatggaatgtggatcgatatgaacgaaccaagcaattttatcagaggttccgaagacggttgccccaacaacgaactggaaaaccctccttatgtgcccggagtcgtgggcggaacattacaggccgcgactatttgcgccagcagccaccaattcctgtccactcactacaacctccacaacctttatggattaaccgaagctattgcaagtcacagggctctggtgaaggctagagggactaggccctttgtgatctcccgatccacctttgccggacacgggagatacgccggtcactggactggtgacgtgtggagctcatgggaacaactggcctcctccgtgccggaaatcttacagttcaaccttctgggtgtccctcttgtcggagcagacgtgtgtgggtttcttggtaacacctccgaggaactgtgtgtgcgctggactcaactgggtgcattctacccattcatgagaaaccacaactccttgctgtccctgccacaagagccctactcgttcagcgagcctgcacaacaggctatgcggaaggcactgaccctgagatacgccctgcttccacacttatacactctcttccatcaagcgcatgtggcaggagaaaccgttgcaaggcctcttttccttgaattccccaaggattcctcgacttggacggtggatcatcagctgctgtggggagaagctctgctgattactccagtgttgcaagccggaaaagctgaggtgaccggatactttccgctgggaacctggtacgacctccagactgtccctgttgaagcccttggatcactgcctccgcctccggcagctccacgcgaaccagctatacattccgagggacagtgggttacattaccagctcctctggacacaatcaacgtccacttaagagctggctacattatccctctgcaaggaccaggactgactacgaccgagagcagacagcagccaatggcactggctgtggctctgaccaagggaggggaagctagaggagaactcttctgggatgatggggagtcccttgaagtgctggaaagaggcgcttacactcaagtcattttccttgcacggaacaacaccattgtgaacgaattggtgcgagtgaccagcgaaggagctggacttcaactgcagaaggtcactgtgctcggagtggctaccgctcctcagcaagtgctgtcgaatggagtccccgtgtcaaactttacctactcccctgacactaaggtgctcgacatttgcgtgtccctcctgatgggagagcagttccttgtgtcctggtgttga

SEQ ID NO: 42 - DNA, 2703 bp, Artificial sequence
  sp7-hGAAwt-delta-43
atggcctttctgtggctgctgagctgttgggccctgctgggcaccaccttcggccaccccgggcggccgcgagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 43 - DNA, 2703 bp, Artificial sequence
  sp7+hGAAco1-delta-43
atggcctttctgtggctgctgagctgttgggccctgctgggcaccaccttcggccaccccggcagacctagagctgtgcctacccagtgtgacgtgccccccaacagcagattcgactgcgcccctgacaaggccatcacccaggaacagtgcgaggccagaggctgctgctacatccctgccaagcagggactgcagggcgctcagatgggacagccctggtgcttcttcccaccctcctaccccagctacaagctggaaaacctgagcagcagcgagatgggctacaccgccaccctgaccagaaccacccccacattcttcccaaaggacatcctgaccctgcggctggacgtgatgatggaaaccgagaaccggctgcacttcaccatcaaggaccccgccaatcggagatacgaggtgcccctggaaaccccccacgtgcactctagagcccccagccctctgtacagcgtggaattcagcgaggaacccttcggcgtgatcgtgcggagacagctggatggcagagtgctgctgaacaccaccgtggcccctctgttcttcgccgaccagttcctgcagctgagcaccagcctgcccagccagtacatcacaggactggccgagcacctgagccccctgatgctgagcacatcctggacccggatcaccctgtggaacagggatctggcccctacccctggcgccaatctgtacggcagccaccctttctacctggccctggaagatggcggatctgcccacggagtgtttctgctgaactccaacgccatggacgtggtgctgcagcctagccctgccctgtcttggagaagcacaggcggcatcctggatgtgtacatctttctgggccccgagcccaagagcgtggtgcagcagtatctggatgtcgtgggctaccccttcatgcccccttactggggcctgggattccacctgtgcagatggggctactccagcaccgccatcaccagacaggtggtggaaaacatgaccagagcccacttcccactggatgtgcagtggaacgacctggactacatggacagcagacgggacttcaccttcaacaaggacggcttccgggacttccccgccatggtgcaggaactgcatcagggcggcagacggtacatgatgatcgtggatcccgccatcagctcctctggccctgccggctcttacagaccctacgacgagggcctgcggagaggcgtgttcatcaccaacgagacaggccagcccctgatcggcaaagtgtggcctggcagcacagccttccccgacttcaccaatcctaccgccctggcttggtgggaggacatggtggccgagttccacgaccaggtgcccttcgacggcatgtggatcgacatgaacgagcccagcaacttcatccggggcagcgaggatggctgccccaacaacgaactggaaaatcccccttacgtgcccggcgtcgtgggcggaacactgcaggccgctacaatctgtgccagcagccaccagtttctgagcacccactacaacctgcacaacctgtacggcctgaccgaggccattgccagccaccgcgctctcgtgaaagccagaggcacacggcccttcgtgatcagcagaagcacctttgccggccacggcagatacgccggacattggactggcgacgtgtggtcctcttgggagcagctggcctctagcgtgcccgagatcctgcagttcaatctgctgggcgtgccactcgtgggcgccgatgtgtgtggcttcctgggcaacacctccgaggaactgtgtgtgcggtggacacagctgggcgccttctaccctttcatgagaaaccacaacagcctgctgagcctgccccaggaaccctacagctttagcgagcctgcacagcaggccatgcggaaggccctgacactgagatacgctctgctgccccacctgtacaccctgtttcaccaggcccatgtggccggcgagacagtggccagacctctgtttctggaattccccaaggacagcagcacctggaccgtggaccatcagctgctgtggggagaggctctgctgattaccccagtgctgcaggcaggcaaggccgaagtgaccggctactttcccctgggcacttggtacgacctgcagaccgtgcctgtggaagccctgggatctctgcctccacctcctgccgctcctagagagcctgccattcactctgagggccagtgggtcacactgcctgcccccctggataccatcaacgtgcacctgagggccggctacatcataccactgcagggacctggcctgaccaccaccgagtctagacagcagccaatggccctggccgtggccctgaccaaaggcggagaagctaggggcgagctgttctgggacgatggcgagagcctggaagtgctggaaagaggcgcctatacccaagtgatcttcctggcccggaacaacaccatcgtgaacgagctggtgcgcgtgacctctgaaggcgctggactgcagctgcagaaagtgaccgtgctgggagtggccacagcccctcagcaggtgctgtctaatggcgtgcccgtgtccaacttcacctacagccccgacaccaaggtgctggacatctgcgtgtcactgctgatgggagagcagtttctggtgtcctggtgctga

SEQ ID NO: 44 - DNA, 2703 bp, Artificial sequence
  sp7+hGAAco2-delta-43
atggcctttctgtggctgctgagctgttgggccctgctgggcaccaccttcggccatcctggtagaccaagagctgtgcctacccaatgcgacgtgccacccaactcccgattcgactgcgcgccagataaggctattacccaagagcagtgtgaagccagaggttgctgctacatcccagcgaagcaaggattgcaaggcgcccaaatgggacaaccttggtgtttcttccccccttcgtacccatcatataaactcgaaaacctgtcctcttcggaaatgggttatactgccaccctcaccagaactactcctactttcttcccgaaagacatcttgaccttgaggctggacgtgatgatggagactgaaaaccggctgcatttcactatcaaagatcctgccaatcggcgatacgaggtccctctggaaacccctcacgtgcactcacgggctccttctccgctttactccgtcgaattctctgaggaacccttcggagtgatcgttagacgccagctggatggtagagtgctgttgaacactactgtggccccacttttcttcgctgaccagtttctgcaactgtccacttccctgccatcccagtacattactggactcgccgaacacctgtcgccactgatgctctcgacctcttggactagaatcactttgtggaacagagacttggcccctactccgggagcaaatctgtacggaagccaccctttttacctggcgctcgaagatggcggatccgctcacggagtgttcctgctgaatagcaacgcaatggacgtggtgctgcaaccttcccctgcactcagttggagaagtaccgggggtattctggacgtgtacatcttcctcggaccagaacccaagagcgtggtgcagcaatatctggacgtggtcggatacccttttatgcctccttactggggactgggattccacctttgccgttggggctactcatccaccgccattaccagacaggtggtggagaatatgaccagagcccacttccctctcgacgtgcagtggaacgatctggactatatggactcccggagagatttcaccttcaacaaggacgggttccgcgattttcccgcgatggttcaagagctccaccagggtggtcgaagatatatgatgatcgtcgacccagccatttcgagcagcggacccgctggatcttatagaccttacgacgaaggccttaggagaggagtgttcatcacaaacgagactggacagcctttgatcggtaaagtgtggcctggatcaaccgcctttcctgactttaccaatcccactgccttggcttggtgggaggacatggtggccgaattccacgaccaagtcccctttgatggaatgtggatcgatatgaacgaaccaagcaattttatcagaggttccgaagacggttgccccaacaacgaactggaaaaccctccttatgtgcccggagtcgtgggcggaacattacaggccgcgactatttgcgccagcagccaccaattcctgtccactcactacaacctccacaacctttatggattaaccgaagctattgcaagtcacagggctctggtgaaggctagagggactaggccctttgtgatctcccgatccacctttgccggacacgggagatacgccggtcactggactggtgacgtgtggagctcatgggaacaactggcctcctccgtgccggaaatcttacagttcaaccttctgggtgtccctcttgtcggagcagacgtgtgtgggtttcttggtaacacctccgaggaactgtgtgtgcgctggactcaactgggtgcattctacccattcatgagaaaccacaactccttgctgtccctgccacaagagccctactcgttcagcgagcctgcacaacaggctatgcggaaggcactgaccctgagatacgccctgcttccacacttatacactctcttccatcaagcgcatgtggcaggagaaaccgttgcaaggcctcttttccttgaattccccaaggattcctcgacttggacggtggatcatcagctgctgtggggagaagctctgctgattactccagtgttgcaagccggaaaagctgaggtgaccggatactttccgctgggaacctggtacgacctccagactgtccctgttgaagcccttggatcactgcctccgcctccggcagctccacgcgaaccagctatacattccgagggacagtgggttacattaccagctcctctggacacaatcaacgtccacttaagagctggctacattatccctctgcaaggaccaggactgactacgaccgagagcagacagcagccaatggcactggctgtggctctgaccaagggaggggaagctagaggagaactcttctgggatgatggggagtcccttgaagtgctggaaagaggcgcttacactcaagtcattttccttgcacggaacaacaccattgtgaacgaattggtgcgagtgaccagcgaaggagctggacttcaactgcagaaggtcactgtgctcggagtggctaccgctcctcagcaagtgctgtcgaatggagtccccgtgtcaaactttacctactcccctgacactaaggtgctcgacatttgcgtgtccctcctgatgggagagcagttccttgtgtcctggtgttga

SEQ ID NO: 45 - DNA, 2691 bp, Artificial sequence
  sp7+hGAAwt-delta-47
atggcctttctgtggctgctgagctgttgggccctgctgggcaccaccttcggcccgcgagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 46 - DNA, 2691 bp, Artificial sequence
  sp7+hGAAco1-delta-47
atggcctttctgtggctgctgagctgttgggccctgctgggcaccaccttcggccctagagctgtgcctacccagtgtgacgtgccccccaacagcagattcgactgcgcccctgacaaggccatcacccaggaacagtgcgaggccagaggctgctgctacatccctgccaagcagggactgcagggcgctcagatgggacagccctggtgcttcttcccaccctcctaccccagctacaagctggaaaacctgagcagcagcgagatgggctacaccgccaccctgaccagaaccacccccacattcttcccaaaggacatcctgaccctgcggctggacgtgatgatggaaaccgagaaccggctgcacttcaccatcaaggaccccgccaatcggagatacgaggtgcccctggaaaccccccacgtgcactctagagcccccagccctctgtacagcgtggaattcagcgaggaacccttcggcgtgatcgtgcggagacagctggatggcagagtgctgctgaacaccaccgtggcccctctgttcttcgccgaccagttcctgcagctgagcaccagcctgcccagccagtacatcacaggactggccgagcacctgagccccctgatgctgagcacatcctggacccggatcaccctgtggaacagggatctggcccctacccctggcgccaatctgtacggcagccaccctttctacctggccctggaagatggcggatctgcccacggagtgtttctgctgaactccaacgccatggacgtggtgctgcagcctagccctgccctgtcttggagaagcacaggcggcatcctggatgtgtacatctttctgggccccgagcccaagagcgtggtgcagcagtatctggatgtcgtgggctaccccttcatgcccccttactggggcctgggattccacctgtgcagatggggctactccagcaccgccatcaccagacaggtggtggaaaacatgaccagagcccacttcccactggatgtgcagtggaacgacctggactacatggacagcagacgggacttcaccttcaacaaggacggcttccgggacttccccgccatggtgcaggaactgcatcagggcggcagacggtacatgatgatcgtggatcccgccatcagctcctctggccctgccggctcttacagaccctacgacgagggcctgcggagaggcgtgttcatcaccaacgagacaggccagcccctgatcggcaaagtgtggcctggcagcacagccttccccgacttcaccaatcctaccgccctggcttggtgggaggacatggtggccgagttccacgaccaggtgcccttcgacggcatgtggatcgacatgaacgagcccagcaacttcatccggggcagcgaggatggctgccccaacaacgaactggaaaatcccccttacgtgcccggcgtcgtgggcggaacactgcaggccgctacaatctgtgccagcagccaccagtttctgagcacccactacaacctgcacaacctgtacggcctgaccgaggccattgccagccaccgcgctctcgtgaaagccagaggcacacggcccttcgtgatcagcagaagcacctttgccggccacggcagatacgccggacattggactggcgacgtgtggtcctcttgggagcagctggcctctagcgtgcccgagatcctgcagttcaatctgctgggcgtgccactcgtgggcgccgatgtgtgtggcttcctgggcaacacctccgaggaactgtgtgtgcggtggacacagctgggcgccttctaccctttcatgagaaaccacaacagcctgctgagcctgccccaggaaccctacagctttagcgagcctgcacagcaggccatgcggaaggccctgacactgagatacgctctgctgccccacctgtacaccctgtttcaccaggcccatgtggccggcgagacagtggccagacctctgtttctggaattccccaaggacagcagcacctggaccgtggaccatcagctgctgtggggagaggctctgctgattaccccagtgctgcaggcaggcaaggccgaagtgaccggctactttcccctgggcacttggtacgacctgcagaccgtgcctgtggaagccctgggatctctgcctccacctcctgccgctcctagagagcctgccattcactctgagggccagtgggtcacactgcctgcccccctggataccatcaacgtgcacctgagggccggctacatcataccactgcagggacctggcctgaccaccaccgagtctagacagcagccaatggccctggccgtggccctgaccaaaggcggagaagctaggggcgagctgttctgggacgatggcgagagcctggaagtgctggaaagaggcgcctatacccaagtgatcttcctggcccggaacaacaccatcgtgaacgagctggtgcgcgtgacctctgaaggcgctggactgcagctgcagaaagtgaccgtgctgggagtggccacagcccctcagcaggtgctgtctaatggcgtgcccgtgtccaacttcacctacagccccgacaccaaggtgctggacatctgcgtgtcactgctgatgggagagcagtttctggtgtcctggtgctga

SEQ ID NO: 47 - DNA, 2691 bp, Artificial sequence
  sp7+hGAAco2-delta-47
atggcctttctgtggctgctgagctgttgggccctgctgggcaccaccttcggcccaagagctgtgcctacccaatgcgacgtgccacccaactcccgattcgactgcgcgccagataaggctattacccaagagcagtgtgaagccagaggttgctgctacatcccagcgaagcaaggattgcaaggcgcccaaatgggacaaccttggtgtttcttccccccttcgtacccatcatataaactcgaaaacctgtcctcttcggaaatgggttatactgccaccctcaccagaactactcctactttcttcccgaaagacatcttgaccttgaggctggacgtgatgatggagactgaaaaccggctgcatttcactatcaaagatcctgccaatcggcgatacgaggtccctctggaaacccctcacgtgcactcacgggctccttctccgctttactccgtcgaattctctgaggaacccttcggagtgatcgttagacgccagctggatggtagagtgctgttgaacactactgtggccccacttttcttcgctgaccagtttctgcaactgtccacttccctgccatcccagtacattactggactcgccgaacacctgtcgccactgatgctctcgacctcttggactagaatcactttgtggaacagagacttggcccctactccgggagcaaatctgtacggaagccaccctttttacctggcgctcgaagatggcggatccgctcacggagtgttcctgctgaatagcaacgcaatggacgtggtgctgcaaccttcccctgcactcagttggagaagtaccgggggtattctggacgtgtacatcttcctcggaccagaacccaagagcgtggtgcagcaatatctggacgtggtcggatacccttttatgcctccttactggggactgggattccacctttgccgttggggctactcatccaccgccattaccagacaggtggtggagaatatgaccagagcccacttccctctcgacgtgcagtggaacgatctggactatatggactcccggagagatttcaccttcaacaaggacgggttccgcgattttcccgcgatggttcaagagctccaccagggtggtcgaagatatatgatgatcgtcgacccagccatttcgagcagcggacccgctggatcttatagaccttacgacgaaggccttaggagaggagtgttcatcacaaacgagactggacagcctttgatcggtaaagtgtggcctggatcaaccgcctttcctgactttaccaatcccactgccttggcttggtgggaggacatggtggccgaattccacgaccaagtcccctttgatggaatgtggatcgatatgaacgaaccaagcaattttatcagaggttccgaagacggttgccccaacaacgaactggaaaaccctccttatgtgcccggagtcgtgggcggaacattacaggccgcgactatttgcgccagcagccaccaattcctgtccactcactacaacctccacaacctttatggattaaccgaagctattgcaagtcacagggctctggtgaaggctagagggactaggccctttgtgatctcccgatccacctttgccggacacgggagatacgccggtcactggactggtgacgtgtggagctcatgggaacaactggcctcctccgtgccggaaatcttacagttcaaccttctgggtgtccctcttgtcggagcagacgtgtgtgggtttcttggtaacacctccgaggaactgtgtgtgcgctggactcaactgggtgcattctacccattcatgagaaaccacaactccttgctgtccctgccacaagagccctactcgttcagcgagcctgcacaacaggctatgcggaaggcactgaccctgagatacgccctgcttccacacttatacactctcttccatcaagcgcatgtggcaggagaaaccgttgcaaggcctcttttccttgaattccccaaggattcctcgacttggacggtggatcatcagctgctgtggggagaagctctgctgattactccagtgttgcaagccggaaaagctgaggtgaccggatactttccgctgggaacctggtacgacctccagactgtccctgttgaagcccttggatcactgcctccgcctccggcagctccacgcgaaccagctatacattccgagggacagtgggttacattaccagctcctctggacacaatcaacgtccacttaagagctggctacattatccctctgcaaggaccaggactgactacgaccgagagcagacagcagccaatggcactggctgtggctctgaccaagggaggggaagctagaggagaactcttctgggatgatggggagtcccttgaagtgctggaaagaggcgcttacactcaagtcattttccttgcacggaacaacaccattgtgaacgaattggtgcgagtgaccagcgaaggagctggacttcaactgcagaaggtcactgtgctcggagtggctaccgctcctcagcaagtgctgtcgaatggagtccccgtgtcaaactttacctactcccctgacactaaggtgctcgacatttgcgtgtccctcctgatgggagagcagttccttgtgtcctggtgttga

SEQ ID NO: 48 - DNA, 2652 bp, Artificial sequence
  hGAAco1-delta-42
gcccaccccggcagacctagagctgtgcctacccagtgtgacgtgccccccaacagcagattcgactgcgcccctgacaaggccatcacccaggaacagtgcgaggccagaggctgctgctacatccctgccaagcagggactgcagggcgctcagatgggacagccctggtgcttcttcccaccctcctaccccagctacaagctggaaaacctgagcagcagcgagatgggctacaccgccaccctgaccagaaccacccccacattcttcccaaaggacatcctgaccctgcggctggacgtgatgatggaaaccgagaaccggctgcacttcaccatcaaggaccccgccaatcggagatacgaggtgcccctggaaaccccccacgtgcactctagagcccccagccctctgtacagcgtggaattcagcgaggaacccttcggcgtgatcgtgcggagacagctggatggcagagtgctgctgaacaccaccgtggcccctctgttcttcgccgaccagttcctgcagctgagcaccagcctgcccagccagtacatcacaggactggccgagcacctgagccccctgatgctgagcacatcctggacccggatcaccctgtggaacagggatctggcccctacccctggcgccaatctgtacggcagccaccctttctacctggccctggaagatggcggatctgcccacggagtgtttctgctgaactccaacgccatggacgtggtgctgcagcctagccctgccctgtcttggagaagcacaggcggcatcctggatgtgtacatctttctgggccccgagcccaagagcgtggtgcagcagtatctggatgtcgtgggctaccccttcatgcccccttactggggcctgggattccacctgtgcagatggggctactccagcaccgccatcaccagacaggtggtggaaaacatgaccagagcccacttcccactggatgtgcagtggaacgacctggactacatggacagcagacgggacttcaccttcaacaaggacggcttccgggacttccccgccatggtgcaggaactgcatcagggcggcagacggtacatgatgatcgtggatcccgccatcagctcctctggccctgccggctcttacagaccctacgacgagggcctgcggagaggcgtgttcatcaccaacgagacaggccagcccctgatcggcaaagtgtggcctggcagcacagccttccccgacttcaccaatcctaccgccctggcttggtgggaggacatggtggccgagttccacgaccaggtgcccttcgacggcatgtggatcgacatgaacgagcccagcaacttcatccggggcagcgaggatggctgccccaacaacgaactggaaaatcccccttacgtgcccggcgtcgtgggcggaacactgcaggccgctacaatctgtgccagcagccaccagtttctgagcacccactacaacctgcacaacctgtacggcctgaccgaggccattgccagccaccgcgctctcgtgaaagccagaggcacacggcccttcgtgatcagcagaagcacctttgccggccacggcagatacgccggacattggactggcgacgtgtggtcctcttgggagcagctggcctctagcgtgcccgagatcctgcagttcaatctgctgggcgtgccactcgtgggcgccgatgtgtgtggcttcctgggcaacacctccgaggaactgtgtgtgcggtggacacagctgggcgccttctaccctttcatgagaaaccacaacagcctgctgagcctgccccaggaaccctacagctttagcgagcctgcacagcaggccatgcggaaggccctgacactgagatacgctctgctgccccacctgtacaccctgtttcaccaggcccatgtggccggcgagacagtggccagacctctgtttctggaattccccaaggacagcagcacctggaccgtggaccatcagctgctgtggggagaggctctgctgattaccccagtgctgcaggcaggcaaggccgaagtgaccggctactttcccctgggcacttggtacgacctgcagaccgtgcctgtggaagccctgggatctctgcctccacctcctgccgctcctagagagcctgccattcactctgagggccagtgggtcacactgcctgcccccctggataccatcaacgtgcacctgagggccggctacatcataccactgcagggacctggcctgaccaccaccgagtctagacagcagccaatggccctggccgtggccctgaccaaaggcggagaagctaggggcgagctgttctgggacgatggcgagagcctggaagtgctggaaagaggcgcctatacccaagtgatcttcctggcccggaacaacaccatcgtgaacgagctggtgcgcgtgacctctgaaggcgctggactgcagctgcagaaagtgaccgtgctgggagtggccacagcccctcagcaggtgctgtctaatggcgtgcccgtgtccaacttcacctacagccccgacaccaaggtgctggacatctgcgtgtcactgctgatgggagagcagtttctggtgtcctggtgctga

SEQ ID NO: 49 - DNA, 2652 bp, Artificial sequence
  hGAAco2-delta-42
gcccatcctggtagaccaagagctgtgcctacccaatgcgacgtgccacccaactcccgattcgactgcgcgccagataaggctattacccaagagcagtgtgaagccagaggttgctgctacatcccagcgaagcaaggattgcaaggcgcccaaatgggacaaccttggtgtttcttccccccttcgtacccatcatataaactcgaaaacctgtcctcttcggaaatgggttatactgccaccctcaccagaactactcctactttcttcccgaaagacatcttgaccttgaggctggacgtgatgatggagactgaaaaccggctgcatttcactatcaaagatcctgccaatcggcgatacgaggtccctctggaaacccctcacgtgcactcacgggctccttctccgctttactccgtcgaattctctgaggaacccttcggagtgatcgttagacgccagctggatggtagagtgctgttgaacactactgtggccccacttttcttcgctgaccagtttctgcaactgtccacttccctgccatcccagtacattactggactcgccgaacacctgtcgccactgatgctctcgacctcttggactagaatcactttgtggaacagagacttggcccctactccgggagcaaatctgtacggaagccaccctttttacctggcgctcgaagatggcggatccgctcacggagtgttcctgctgaatagcaacgcaatggacgtggtgctgcaaccttcccctgcactcagttggagaagtaccgggggtattctggacgtgtacatcttcctcggaccagaacccaagagcgtggtgcagcaatatctggacgtggtcggatacccttttatgcctccttactggggactgggattccacctttgccgttggggctactcatccaccgccattaccagacaggtggtggagaatatgaccagagcccacttccctctcgacgtgcagtggaacgatctggactatatggactcccggagagatttcaccttcaacaaggacgggttccgcgattttcccgcgatggttcaagagctccaccagggtggtcgaagatatatgatgatcgtcgacccagccatttcgagcagcggacccgctggatcttatagaccttacgacgaaggccttaggagaggagtgttcatcacaaacgagactggacagcctttgatcggtaaagtgtggcctggatcaaccgcctttcctgactttaccaatcccactgccttggcttggtgggaggacatggtggccgaattccacgaccaagtcccctttgatggaatgtggatcgatatgaacgaaccaagcaattttatcagaggttccgaagacggttgccccaacaacgaactggaaaaccctccttatgtgcccggagtcgtgggcggaacattacaggccgcgactatttgcgccagcagccaccaattcctgtccactcactacaacctccacaacctttatggattaaccgaagctattgcaagtcacagggctctggtgaaggctagagggactaggccctttgtgatctcccgatccacctttgccggacacgggagatacgccggtcactggactggtgacgtgtggagctcatgggaacaactggcctcctccgtgccggaaatcttacagttcaaccttctgggtgtccctcttgtcggagcagacgtgtgtgggtttcttggtaacacctccgaggaactgtgtgtgcgctggactcaactgggtgcattctacccattcatgagaaaccacaactccttgctgtccctgccacaagagccctactcgttcagcgagcctgcacaacaggctatgcggaaggcactgaccctgagatacgccctgcttccacacttatacactctcttccatcaagcgcatgtggcaggagaaaccgttgcaaggcctcttttccttgaattccccaaggattcctcgacttggacggtggatcatcagctgctgtggggagaagctctgctgattactccagtgttgcaagccggaaaagctgaggtgaccggatactttccgctgggaacctggtacgacctccagactgtccctgttgaagcccttggatcactgcctccgcctccggcagctccacgcgaaccagctatacattccgagggacagtgggttacattaccagctcctctggacacaatcaacgtccacttaagagctggctacattatccctctgcaaggaccaggactgactacgaccgagagcagacagcagccaatggcactggctgtggctctgaccaagggaggggaagctagaggagaactcttctgggatgatggggagtcccttgaagtgctggaaagaggcgcttacactcaagtcattttccttgcacggaacaacaccattgtgaacgaattggtgcgagtgaccagcgaaggagctggacttcaactgcagaaggtcactgtgctcggagtggctaccgctcctcagcaagtgctgtcgaatggagtccccgtgtcaaactttacctactcccctgacactaaggtgctcgacatttgcgtgtccctcctgatgggagagcagttccttgtgtcctggtgttga

SEQ ID NO: 50 - DNA, 2649 bp, Artificial sequence
  hGAAco1-delta-43
caccccggcagacctagagctgtgcctacccagtgtgacgtgccccccaacagcagattcgactgcgcccctgacaaggccatcacccaggaacagtgcgaggccagaggctgctgctacatccctgccaagcagggactgcagggcgctcagatgggacagccctggtgcttcttcccaccctcctaccccagctacaagctggaaaacctgagcagcagcgagatgggctacaccgccaccctgaccagaaccacccccacattcttcccaaaggacatcctgaccctgcggctggacgtgatgatggaaaccgagaaccggctgcacttcaccatcaaggaccccgccaatcggagatacgaggtgcccctggaaaccccccacgtgcactctagagcccccagccctctgtacagcgtggaattcagcgaggaacccttcggcgtgatcgtgcggagacagctggatggcagagtgctgctgaacaccaccgtggcccctctgttcttcgccgaccagttcctgcagctgagcaccagcctgcccagccagtacatcacaggactggccgagcacctgagccccctgatgctgagcacatcctggacccggatcaccctgtggaacagggatctggcccctacccctggcgccaatctgtacggcagccaccctttctacctggccctggaagatggcggatctgcccacggagtgtttctgctgaactccaacgccatggacgtggtgctgcagcctagccctgccctgtcttggagaagcacaggcggcatcctggatgtgtacatctttctgggccccgagcccaagagcgtggtgcagcagtatctggatgtcgtgggctaccccttcatgcccccttactggggcctgggattccacctgtgcagatggggctactccagcaccgccatcaccagacaggtggtggaaaacatgaccagagcccacttcccactggatgtgcagtggaacgacctggactacatggacagcagacgggacttcaccttcaacaaggacggcttccgggacttccccgccatggtgcaggaactgcatcagggcggcagacggtacatgatgatcgtggatcccgccatcagctcctctggccctgccggctcttacagaccctacgacgagggcctgcggagaggcgtgttcatcaccaacgagacaggccagcccctgatcggcaaagtgtggcctggcagcacagccttccccgacttcaccaatcctaccgccctggcttggtgggaggacatggtggccgagttccacgaccaggtgcccttcgacggcatgtggatcgacatgaacgagcccagcaacttcatccggggcagcgaggatggctgccccaacaacgaactggaaaatcccccttacgtgcccggcgtcgtgggcggaacactgcaggccgctacaatctgtgccagcagccaccagtttctgagcacccactacaacctgcacaacctgtacggcctgaccgaggccattgccagccaccgcgctctcgtgaaagccagaggcacacggcccttcgtgatcagcagaagcacctttgccggccacggcagatacgccggacattggactggcgacgtgtggtcctcttgggagcagctggcctctagcgtgcccgagatcctgcagttcaatctgctgggcgtgccactcgtgggcgccgatgtgtgtggcttcctgggcaacacctccgaggaactgtgtgtgcggtggacacagctgggcgccttctaccctttcatgagaaaccacaacagcctgctgagcctgccccaggaaccctacagctttagcgagcctgcacagcaggccatgcggaaggccctgacactgagatacgctctgctgccccacctgtacaccctgtttcaccaggcccatgtggccggcgagacagtggccagacctctgtttctggaattccccaaggacagcagcacctggaccgtggaccatcagctgctgtggggagaggctctgctgattaccccagtgctgcaggcaggcaaggccgaagtgaccggctactttcccctgggcacttggtacgacctgcagaccgtgcctgtggaagccctgggatctctgcctccacctcctgccgctcctagagagcctgccattcactctgagggccagtgggtcacactgcctgcccccctggataccatcaacgtgcacctgagggccggctacatcataccactgcagggacctggcctgaccaccaccgagtctagacagcagccaatggccctggccgtggccctgaccaaaggcggagaagctaggggcgagctgttctgggacgatggcgagagcctggaagtgctggaaagaggcgcctatacccaagtgatcttcctggcccggaacaacaccatcgtgaacgagctggtgcgcgtgacctctgaaggcgctggactgcagctgcagaaagtgaccgtgctgggagtggccacagcccctcagcaggtgctgtctaatggcgtgcccgtgtccaacttcacctacagccccgacaccaaggtgctggacatctgcgtgtcactgctgatgggagagcagtttctggtgtcctggtgctga

SEQ ID NO: 51 - DNA, 2649 bp, Artificial sequence
  hGAAco2-delta-43
catcctggtagaccaagagctgtgcctacccaatgcgacgtgccacccaactcccgattcgactgcgcgccagataaggctattacccaagagcagtgtgaagccagaggttgctgctacatcccagcgaagcaaggattgcaaggcgcccaaatgggacaaccttggtgtttcttccccccttcgtacccatcatataaactcgaaaacctgtcctcttcggaaatgggttatactgccaccctcaccagaactactcctactttcttcccgaaagacatcttgaccttgaggctggacgtgatgatggagactgaaaaccggctgcatttcactatcaaagatcctgccaatcggcgatacgaggtccctctggaaacccctcacgtgcactcacgggctccttctccgctttactccgtcgaattctctgaggaacccttcggagtgatcgttagacgccagctggatggtagagtgctgttgaacactactgtggccccacttttcttcgctgaccagtttctgcaactgtccacttccctgccatcccagtacattactggactcgccgaacacctgtcgccactgatgctctcgacctcttggactagaatcactttgtggaacagagacttggcccctactccgggagcaaatctgtacggaagccaccctttttacctggcgctcgaagatggcggatccgctcacggagtgttcctgctgaatagcaacgcaatggacgtggtgctgcaaccttcccctgcactcagttggagaagtaccgggggtattctggacgtgtacatcttcctcggaccagaacccaagagcgtggtgcagcaatatctggacgtggtcggatacccttttatgcctccttactggggactgggattccacctttgccgttggggctactcatccaccgccattaccagacaggtggtggagaatatgaccagagcccacttccctctcgacgtgcagtggaacgatctggactatatggactcccggagagatttcaccttcaacaaggacgggttccgcgattttcccgcgatggttcaagagctccaccagggtggtcgaagatatatgatgatcgtcgacccagccatttcgagcagcggacccgctggatcttatagaccttacgacgaaggccttaggagaggagtgttcatcacaaacgagactggacagcctttgatcggtaaagtgtggcctggatcaaccgcctttcctgactttaccaatcccactgccttggcttggtgggaggacatggtggccgaattccacgaccaagtcccctttgatggaatgtggatcgatatgaacgaaccaagcaattttatcagaggttccgaagacggttgccccaacaacgaactggaaaaccctccttatgtgcccggagtcgtgggcggaacattacaggccgcgactatttgcgccagcagccaccaattcctgtccactcactacaacctccacaacctttatggattaaccgaagctattgcaagtcacagggctctggtgaaggctagagggactaggccctttgtgatctcccgatccacctttgccggacacgggagatacgccggtcactggactggtgacgtgtggagctcatgggaacaactggcctcctccgtgccggaaatcttacagttcaaccttctgggtgtccctcttgtcggagcagacgtgtgtgggtttcttggtaacacctccgaggaactgtgtgtgcgctggactcaactgggtgcattctacccattcatgagaaaccacaactccttgctgtccctgccacaagagccctactcgttcagcgagcctgcacaacaggctatgcggaaggcactgaccctgagatacgccctgcttccacacttatacactctcttccatcaagcgcatgtggcaggagaaaccgttgcaaggcctcttttccttgaattccccaaggattcctcgacttggacggtggatcatcagctgctgtggggagaagctctgctgattactccagtgttgcaagccggaaaagctgaggtgaccggatactttccgctgggaacctggtacgacctccagactgtccctgttgaagcccttggatcactgcctccgcctccggcagctccacgcgaaccagctatacattccgagggacagtgggttacattaccagctcctctggacacaatcaacgtccacttaagagctggctacattatccctctgcaaggaccaggactgactacgaccgagagcagacagcagccaatggcactggctgtggctctgaccaagggaggggaagctagaggagaactcttctgggatgatggggagtcccttgaagtgctggaaagaggcgcttacactcaagtcattttccttgcacggaacaacaccattgtgaacgaattggtgcgagtgaccagcgaaggagctggacttcaactgcagaaggtcactgtgctcggagtggctaccgctcctcagcaagtgctgtcgaatggagtccccgtgtcaaactttacctactcccctgacactaaggtgctcgacatttgcgtgtccctcctgatgggagagcagttccttgtgtcctggtgttga

SEQ ID NO: 52 - DNA, 2637 bp, Artificial sequence
  hGAAco1-delta-47
cctagagctgtgcctacccagtgtgacgtgccccccaacagcagattcgactgcgcccctgacaaggccatcacccaggaacagtgcgaggccagaggctgctgctacatccctgccaagcagggactgcagggcgctcagatgggacagccctggtgcttcttcccaccctcctaccccagctacaagctggaaaacctgagcagcagcgagatgggctacaccgccaccctgaccagaaccacccccacattcttcccaaaggacatcctgaccctgcggctggacgtgatgatggaaaccgagaaccggctgcacttcaccatcaaggaccccgccaatcggagatacgaggtgcccctggaaaccccccacgtgcactctagagcccccagccctctgtacagcgtggaattcagcgaggaacccttcggcgtgatcgtgcggagacagctggatggcagagtgctgctgaacaccaccgtggcccctctgttcttcgccgaccagttcctgcagctgagcaccagcctgcccagccagtacatcacaggactggccgagcacctgagccccctgatgctgagcacatcctggacccggatcaccctgtggaacagggatctggcccctacccctggcgccaatctgtacggcagccaccctttctacctggccctggaagatggcggatctgcccacggagtgtttctgctgaactccaacgccatggacgtggtgctgcagcctagccctgccctgtcttggagaagcacaggcggcatcctggatgtgtacatctttctgggccccgagcccaagagcgtggtgcagcagtatctggatgtcgtgggctaccccttcatgcccccttactggggcctgggattccacctgtgcagatggggctactccagcaccgccatcaccagacaggtggtggaaaacatgaccagagcccacttcccactggatgtgcagtggaacgacctggactacatggacagcagacgggacttcaccttcaacaaggacggcttccgggacttccccgccatggtgcaggaactgcatcagggcggcagacggtacatgatgatcgtggatcccgccatcagctcctctggccctgccggctcttacagaccctacgacgagggcctgcggagaggcgtgttcatcaccaacgagacaggccagcccctgatcggcaaagtgtggcctggcagcacagccttccccgacttcaccaatcctaccgccctggcttggtgggaggacatggtggccgagttccacgaccaggtgcccttcgacggcatgtggatcgacatgaacgagcccagcaacttcatccggggcagcgaggatggctgccccaacaacgaactggaaaatcccccttacgtgcccggcgtcgtgggcggaacactgcaggccgctacaatctgtgccagcagccaccagtttctgagcacccactacaacctgcacaacctgtacggcctgaccgaggccattgccagccaccgcgctctcgtgaaagccagaggcacacggcccttcgtgatcagcagaagcacctttgccggccacggcagatacgccggacattggactggcgacgtgtggtcctcttgggagcagctggcctctagcgtgcccgagatcctgcagttcaatctgctgggcgtgccactcgtgggcgccgatgtgtgtggcttcctgggcaacacctccgaggaactgtgtgtgcggtggacacagctgggcgccttctaccctttcatgagaaaccacaacagcctgctgagcctgccccaggaaccctacagctttagcgagcctgcacagcaggccatgcggaaggccctgacactgagatacgctctgctgccccacctgtacaccctgtttcaccaggcccatgtggccggcgagacagtggccagacctctgtttctggaattccccaaggacagcagcacctggaccgtggaccatcagctgctgtggggagaggctctgctgattaccccagtgctgcaggcaggcaaggccgaagtgaccggctactttcccctgggcacttggtacgacctgcagaccgtgcctgtggaagccctgggatctctgcctccacctcctgccgctcctagagagcctgccattcactctgagggccagtgggtcacactgcctgcccccctggataccatcaacgtgcacctgagggccggctacatcataccactgcagggacctggcctgaccaccaccgagtctagacagcagccaatggccctggccgtggccctgaccaaaggcggagaagctaggggcgagctgttctgggacgatggcgagagcctggaagtgctggaaagaggcgcctatacccaagtgatcttcctggcccggaacaacaccatcgtgaacgagctggtgcgcgtgacctctgaaggcgctggactgcagctgcagaaagtgaccgtgctgggagtggccacagcccctcagcaggtgctgtctaatggcgtgcccgtgtccaacttcacctacagccccgacaccaaggtgctggacatctgcgtgtcactgctgatgggagagcagtttctggtgtcctggtgctga

SEQ ID NO: 53 - DNA, 2637 bp, Artificial sequence
  hGAAco2-delta-47
ccaagagctgtgcctacccaatgcgacgtgccacccaactcccgattcgactgcgcgccagataaggctattacccaagagcagtgtgaagccagaggttgctgctacatcccagcgaagcaaggattgcaaggcgcccaaatgggacaaccttggtgtttcttccccccttcgtacccatcatataaactcgaaaacctgtcctcttcggaaatgggttatactgccaccctcaccagaactactcctactttcttcccgaaagacatcttgaccttgaggctggacgtgatgatggagactgaaaaccggctgcatttcactatcaaagatcctgccaatcggcgatacgaggtccctctggaaacccctcacgtgcactcacgggctccttctccgctttactccgtcgaattctctgaggaacccttcggagtgatcgttagacgccagctggatggtagagtgctgttgaacactactgtggccccacttttcttcgctgaccagtttctgcaactgtccacttccctgccatcccagtacattactggactcgccgaacacctgtcgccactgatgctctcgacctcttggactagaatcactttgtggaacagagacttggcccctactccgggagcaaatctgtacggaagccaccctttttacctggcgctcgaagatggcggatccgctcacggagtgttcctgctgaatagcaacgcaatggacgtggtgctgcaaccttcccctgcactcagttggagaagtaccgggggtattctggacgtgtacatcttcctcggaccagaacccaagagcgtggtgcagcaatatctggacgtggtcggatacccttttatgcctccttactggggactgggattccacctttgccgttggggctactcatccaccgccattaccagacaggtggtggagaatatgaccagagcccacttccctctcgacgtgcagtggaacgatctggactatatggactcccggagagatttcaccttcaacaaggacgggttccgcgattttcccgcgatggttcaagagctccaccagggtggtcgaagatatatgatgatcgtcgacccagccatttcgagcagcggacccgctggatcttatagaccttacgacgaaggccttaggagaggagtgttcatcacaaacgagactggacagcctttgatcggtaaagtgtggcctggatcaaccgcctttcctgactttaccaatcccactgccttggcttggtgggaggacatggtggccgaattccacgaccaagtcccctttgatggaatgtggatcgatatgaacgaaccaagcaattttatcagaggttccgaagacggttgccccaacaacgaactggaaaaccctccttatgtgcccggagtcgtgggcggaacattacaggccgcgactatttgcgccagcagccaccaattcctgtccactcactacaacctccacaacctttatggattaaccgaagctattgcaagtcacagggctctggtgaaggctagagggactaggccctttgtgatctcccgatccacctttgccggacacgggagatacgccggtcactggactggtgacgtgtggagctcatgggaacaactggcctcctccgtgccggaaatcttacagttcaaccttctgggtgtccctcttgtcggagcagacgtgtgtgggtttcttggtaacacctccgaggaactgtgtgtgcgctggactcaactgggtgcattctacccattcatgagaaaccacaactccttgctgtccctgccacaagagccctactcgttcagcgagcctgcacaacaggctatgcggaaggcactgaccctgagatacgccctgcttccacacttatacactctcttccatcaagcgcatgtggcaggagaaaccgttgcaaggcctcttttccttgaattccccaaggattcctcgacttggacggtggatcatcagctgctgtggggagaagctctgctgattactccagtgttgcaagccggaaaagctgaggtgaccggatactttccgctgggaacctggtacgacctccagactgtccctgttgaagcccttggatcactgcctccgcctccggcagctccacgcgaaccagctatacattccgagggacagtgggttacattaccagctcctctggacacaatcaacgtccacttaagagctggctacattatccctctgcaaggaccaggactgactacgaccgagagcagacagcagccaatggcactggctgtggctctgaccaagggaggggaagctagaggagaactcttctgggatgatggggagtcccttgaagtgctggaaagaggcgcttacactcaagtcattttccttgcacggaacaacaccattgtgaacgaattggtgcgagtgaccagcgaaggagctggacttcaactgcagaaggtcactgtgctcggagtggctaccgctcctcagcaagtgctgtcgaatggagtccccgtgtcaaactttacctactcccctgacactaaggtgctcgacatttgcgtgtccctcctgatgggagagcagttccttgtgtcctggtgttga

SEQ ID NO: 54 - DNA, 54 bp, Artificial Sequence
  sp7
atggcctttctgtggctgctgagctgttgggccctgctgggcaccaccttcggc

SEQ ID NO: 55 - DNA, 1121 bp, Artificial Sequence
  LiMP
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggaccccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatct

(19) United States
(12) Patent Application Publication
(10) Pub. No.: US 2022/0331408 A1
(43) Pub. Date: Oct. 20, 2022

(54) TREATMENT OF GLYCOGEN STORAGE DISEASE (GSD)

(71) Applicants: GENETHON, EVRY (FR); INSTITUT NATIONAL DE LA SANTE ET DE LA RECHERCHE MEDICALE (INSERM), PARIS (FR); UNIVERSITE D'EVRY VAL D'ESSONNE, EVRY (FR)

(72) Inventors: Federico MINGOZZI, Paris (FR); Giuseppe RONZITTI, Fontainebleau (FR); Fanny COLLAUD, Paris (FR)

(21) Appl. No.: 17/625,440

(22) PCT Filed: Jul. 9, 2020
(86) PCT No.: PCT/EP2020/069432

§ 371 Date: Jan. 7, 2022

(30) Foreign Application Priority Data
Jul. 9, 2019 (EP) 19305928.4

(51) Int. Cl. A61K 38/47; A61K 31/44; A61K 31/137; A61P 3/00

(52) U.S. Cl. CPC A61K 38/47; A61K 31/44; A61K 31/137; A61P 3/00; A61K 48/00

(57) ABSTRACT

The invention relates to a kit of parts comprising (i) pharmacological chaperones or a pharmaceutically acceptable salt thereof and (ii) a therapeutic acid-alpha glucosidase (GAA) polypeptide or a nucleic acid molecule encoding a therapeutic GAA polypeptide, wherein said pharmacological chaperones are 1-deoxynojirimycin (DNJ) or a derivative thereof and ambroxol (ABX) or a derivative thereof.

### CROSS REFERENCE TO RELATED APPLICATIONS

This application is a U.S. National Stage Application of PCT/EP2020/069432 filed 9 Jul. 2020, which claims priority to European Patent Application No. 19305928.4 filed on 9 Jul. 2019, the entire disclosures of which are hereby incorporated by reference in their entireties.

### SEQUENCE LISTING

The instant application contains a Sequence Listing which has been submitted electronically in ASCII format and is hereby incorporated by reference in its entirety. Said ASCII copy, created on 7 Jan. 2022, is named 0177_0186_ST25.txt and is 213 bytes in size.

### BACKGROUND OF THE INVENTION

Glycogen storage disease (GSD) is a metabolic disorder caused by enzyme deficiencies affecting glycogen synthesis, glycogen breakdown or glycolysis, typically within muscles and/or liver cells. GSD is classified in different types, from GSD type 0 to GSD type XV (Table 1).

TABLE 1
GSD types

| Type | Common name | Enzyme deficiency (Gene) |
|---|---|---|
| GSD 0 | | Glycogen synthase |
| | | (GYS2) |
| GSD I | von Gierke's disease | Glucose-6-phosphatase |
| | | (G6PC/SLC37A4) |
| GSD II | Pompe's disease | Acid alpha-glucosidase |
| | | (GAA) |
| GSD III | Cori's disease or | Glycogen debranching enzyme |
| | Forbes' disease | (AGL) |
| GSD IV | Andersen disease | Glycogen branching enzyme |
| | | (GBE1) |
| GSD V | McArdle disease | Muscle glycogen phosphorylase |
| | | (PYGM) |
| GSD VI | Hers' disease | Liver glycogen phosphorylase |
| | | (PYGL) |
| | | Muscle phosphoglycerate mutase |
| | | (PGAM2) |
| GSD VII | Tarui's disease | Muscle phosphofructokinase |
| | | (PKFM) |
| GSD IX | | Phosphorylase kinase |
| | | (PHKA2/PHKB/PHKG2/PHKA1) |
| GSD X | | Phosphoglycerate mutase |
| | | (PGAM2) |
| GSD XI | | Muscle lactate dehydrogenase |
| | | (LDHA) |
| GSD XII | Aldolase A | Aldolase A |
| | deficiency | (ALDOA) |
| GSD XIII | | β-enolase |
| | | (ENO3) |
| GSD XV | | Glycogenin-1 |
| | | (GYG1) |

Pompe disease, also known as GSD II or acid maltase deficiency, is an autosomal recessive metabolic myopathy caused by a deficiency of the lysosomal enzyme acid alpha-glucosidase (GAA). GAA is an exo-1,4 and 1,6-α-glucosidase that hydrolyzes glycogen to glucose in the lysosome. Deficiency of GAA leads to glycogen accumulation in lysosomes and causes progressive damage to respiratory, cardiac, and skeletal muscle. The disease ranges from a rapidly progressive infantile course that is usually fatal by 1-2 years of age to a more slowly progressive and heterogeneous course that causes significant morbidity and early mortality in children and adults [1] and [2].

Central nervous system (CNS) dysfunctions, in particular respiratory neuromuscular dysfunction, are prominent in GSD, particularly in early and late onset Pompe disease patients. Studies showed that glycogen accumulation in central nervous system causes respiratory dysfunction in patients suffering from Pompe disease, leading to respiratory impairments at 4-6 months age [3] and [4]. It is therefore important to develop therapies that improve correction of the respiratory dysfunction in Pompe disease, in particular therapies that improve correction of the pathogenic accumulation of glycogen in a tissue of the nervous system of GSD patients.

Current human therapy for treating Pompe disease involves administration of recombinant human GAA, otherwise termed enzyme-replacement therapy (ERT). ERT has demonstrated efficacy for severe, infantile GSD II. However the benefit of ERT is limited by the need for frequent infusions and the development of inhibitor antibodies against recombinant hGAA [5]. Furthermore, ERT does not correct efficiently the entire body, probably because of a combination of poor biodistribution of the protein following peripheral vein delivery, lack of uptake from several tissues, and high immunogenicity. Especially, it has been shown that ERT is less effective for treating CNS dysfunctions, especially to improve correction of the accumulation of glycogen in a tissue of the nervous system of GSD patients, since the recombinant GAA does not cross the blood brain [6] and [7].

Gene therapy approaches have also been investigated to treat Pompe disease. For example, WO2018/046774 discloses the use of a nucleic acid molecule encoding a truncated GAA polypeptide fused with a signal peptide, for improving the tissue uptake of GAA. However, glycogen accumulation was only partially rescued in the central nervous system.

Another approach developed for treating Pompe disease is the administration of a pharmacological chaperone for facilitating the folding and enhancing the stability of GAA. Several pharmacological chaperones have been tested, for example 1-deoxynojirimycin (DNJ) or a derivative thereof (WO2006/125141), in particular a derivative of DNJ named NB-DNJ (AT2221 or miglustat). Miglustat has also been tested in combination with a recombinant GAA polypeptide to promote GAA activity and improve muscle function [8]. However, DNJ or its derivatives did not increase the uptake of GAA in a tissue of the nervous system.

ABX or a combination of ABX and DNJ have also been tested on Pompe cells model expressing mutant forms of GAA [9]. However, ABX or ABX and DNJ did not increase the tissue uptake of GAA, especially in a tissue of the nervous system.

Therefore, there is still a need to provide better therapies for treating GSD, such as Pompe disease. In particular, there is a need to provide better therapies for increasing the uptake of GAA in a tissue of the nervous system, for treating CNS dysfunctions in GSD, for improving the respiratory neuromuscular function and/or for decreasing respiratory impairments in a subject having a GSD.

### SUMMARY OF THE INVENTION

In a first aspect, the invention relates to kit of parts comprising (i) pharmacological chaperones or a pharmaceutically acceptable salt thereof and (ii) a therapeutic acid-alpha glucosidase (GAA) polypeptide or a nucleic acid molecule encoding a therapeutic GAA polypeptide, wherein said pharmacological chaperones are 1-deoxynojirimycin (DNJ) or a derivative thereof and ambroxol (ABX) or a derivative thereof. The kit of parts according to the invention may be used:
as a medicament,
in the treatment of glycogen storage disease (GSD), and/or
in a method for treating the central nervous system (CNS) dysfunctions of GSD.

In a second aspect, the invention relates to a composition comprising pharmacological chaperones or a pharmaceutically acceptable salt thereof, for use:
in the treatment of glycogen storage disease (GSD) in a subject receiving a therapeutic acid-alpha glucosidase (GAA) treatment for treating said GSD,
in a method of increasing the uptake of GAA in a tissue of the nervous system in a subject receiving a therapeutic GAA treatment for treating a GSD, and/or
in a method for treating the central nervous system (CNS) dysfunctions of GSD in a subject receiving a therapeutic GAA treatment for treating a GSD.
   wherein said pharmacological chaperones are DNJ or a derivative thereof and ABX or a derivative thereof.

### LEGENDS TO THE FIGURES

FIG. 1 represents the experimental design of the study on WT mice. 6-8-week-old C57Bl/6J male mice were intravenously injected with PBS or an AAV8 vector expressing secretable GAA (AAV-GAA) at 5×10¹¹ vg/kg. Two months after vector injection, mice were left untreated or treated for four weeks with seven chaperone molecules or combination of them in drinking water. Each week of treatment consisted in three days under treatment and four days off (Wash-out). Three months after gene therapy treatment, mice were bled and sacrificed to harvest tissues.

FIG. 2 shows that chaperone treatment results in improved circulating GAA levels in wild-type mice. Three months after vector injection, the levels of GAA in blood were measured by Western-blot in mice treated as described in FIG. 1. Data were expressed as the ratio between the human GAA band intensity and a non-specific band intensity used for normalization. Error bars represent the standard deviation of the mean. Statistical analysis was performed by ANOVA (*=p<0.05 vs. AAV-GAA injected mice who received normal water during the third month, n=5 per group except for DNJ-ABX treated group (n=3), VOGLIBOSE and ACARBOSE treated group (n=4), CTRL represents mice injected with PBS and not receiving AAV-GAA).

FIG. 3 shows that chaperone treatment results in improved GAA uptake in tissues of wild-type mice. Three months after vector injection, the levels of lysosomal GAA were measured by Western-blot in heart (FIG. 3A), triceps (FIG. 3B), diaphragm (FIG. 3C), quadriceps (FIG. 3D), brain (FIG. 3E) and spinal cord (FIG. 3F) of mice treated as described in FIG. 1. Data were expressed as the ratio between the human GAA band intensity and GAPDH intensity used for normalization. Error bars represent the standard deviation of the mean. Statistical analyses were performed by ANOVA (*=p<0.05 vs. AAV-GAA injected mice who received normal water during the third month, n=5 per group, except for DNJ-ABX treated group (n=3), VOGLIBOSE and ACARBOSE treated group (n=4)).

FIG. 4 represents the experimental design of the study on GAA deficient (Knock-out) mice. Three to four-month-old GAA deficient male mice were intravenously injected with PBS or an AAV8 vector expressing secretable GAA (AAV-GAA) at 1×10¹¹ vg/kg in combination with pharmacological chaperones (PC) dissolved in the drinking water. Untreated GAA wild-type mice and two groups of GAA deficient mice injected with the AAV-GAA vector or PBS were used as controls. PC molecules were orally administered to the mice, using a “3 days on/4 days off” regimen, consisting in three consecutive days of treatment followed by four consecutive days with drinking water only. Two months after gene therapy treatment in combination with the pharmacological chaperones, mice were bled and sacrificed to harvest tissues.

FIG. 5 shows that chaperone treatment results in improved circulating GAA levels in GAA KO mice. Two months after vector injection, the levels of GAA in blood were measured by Western-blot in mice treated as described in FIG. 4. Data were expressed as the ratio between the human GAA band intensity and a non-specific band intensity used for normalization. Error bars represent the standard deviation of the mean. Statistical analysis was performed by ANOVA (*=p<0.05 vs. AAV-GAA injected mice who received normal water during the two month, n=8 per group except for ABX treated group (n=7), GAA^{+/+} mice (WT) and GAA^{-/-} mice (KO) were injected with PBS and received normal water).

FIG. 6 shows that chaperone treatment results in improved circulating GAA activity in GAA KO mice. Two months after vector injection, the GAA activity in blood was measured in blood of mice treated as described in FIG. 4. Error bars represent the standard deviation of the mean. Statistical analysis was performed by ANOVA (*=p<0.05 vs. AAV-GAA injected mice who received normal water during the two month, n=8 per group except for ABX treated group (n=7), GAA^{+/+} mice (WT) and GAA^{-/-} mice (KO) were injected with PBS and received normal water).

FIG. 7 shows that chaperone treatment results in improved GAA uptake in tissues of GAA KO mice. Two months after vector injection, mice treated as described in FIG. 4 were sacrificed and tissues were collected. Results of GAA activity detected in heart (FIG. 7A), diaphragm (FIG. 7B), triceps (FIG. 7C) and quadriceps (FIG. 7D) are represented. Error bars represent the standard deviation of the mean. Statistical analyses were performed by ANOVA (*=p<0.05 vs. AAV-GAA injected mice who received normal water during the third month, n=8 per group, except for ABX treated group (n=7)).

FIG. 8 shows that chaperone treatment results in reduced glycogen accumulation in tissues of GAA KO mice. Two months after vector injection, mice treated as described in FIG. 4 were sacrificed and tissues were collected. Glycogen content was measured in heart (FIG. 8A), diaphragm (FIG. 8B), triceps (FIG. 8C) and quadriceps (FIG. 8D). Error bars represent the standard deviation of the mean. Statistical analyses were performed by ANOVA (*=p<0.05 vs. AAV-GAA injected mice who received normal water during the third month, n=8 per group, except for ABX treated group (n=7)).

FIG. 9 represents the experimental design of the study on GAA deficient (Knock-out) mice. Three to four-month-old GAA deficient male mice were treated intravenously by ERT (alglucosidase alpha, 20 mg/kg) in combination with pharmacological chaperones (PC) dissolved in the drinking water. Untreated GAA wild-type mice, untreated GAA deficient mice and GAA deficient mice treated by ERT only were used as controls. Blood sampling was performed three hours post ERT.

FIG. 10 shows that chaperone treatment results in improved circulating GAA levels and activity in GAA KO mice. Three hours after ERT, the GAA levels (FIG. 10A) and activity (FIG. 10B) in blood were measured in mice treated as described in FIG. 9. GAA levels results were expressed as the ratio between the human GAA band intensity and a non-specific band intensity used for normalization. Error bars represent the standard deviation of the mean. Statistical analysis was performed by ANOVA (*=p<0.05 vs. ERT-mice who received normal water during the protocol, n=8 per group, untreated GAA^{+/+} mice (WT) and GAA^{-/-} mice (KO) received normal water).

### DETAILED DESCRIPTION OF THE INVENTION

### Definitions

The term “pharmacological chaperone” refers to a small molecule that stabilizes an already folded protein by binding to it and stabilizing it against thermal denaturation and proteolytic degradation. According to the present invention, a pharmacological chaperone may be in the form of a salt, e.g. a pharmaceutically acceptable salt.

The term “pharmacological chaperones” according to the invention means both (i) 1-deoxynojirimycin (DNJ) or a derivative thereof and (ii) ambroxol (ABX) or a derivative thereof.

The term “pharmaceutically acceptable salt” refers to salts of acids or bases, known for their use in the preparation of active principles for their use in therapy. Examples of pharmaceutically acceptable acids suitable as source of anions are those disclosed in the Handbook of Pharmaceutical Salts: Properties, Selection and Use (P. H. Stahl and C. G. Wermuth, Weinheim/ZOrich:Wiley-VCH/VHCA, 200). Salts that are approved by a regulatory agency of the Federal or a state government or listed in the U.S. or European Pharmacopeia or other generally recognized pharmacopeia for use in animals, and humans. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

The term “DNJ” according to the invention means 1-deoxynojirimycin (CAS Number 19130-96-2, INN duvoglustat). The term “DNJ derivative” according to the invention means a compound derived from DNJ. A DNJ derivative may be selected from N-methyl-DNJ, N-butyl-DNJ, N-cyclopropylmethyl-DNJ, N-(2-(N,N-dimethylamido)ethyloxy-DNJ, N-4-t-butyloxycarbonyl-piperidnylmethyl-DNJ, N-2-R-tetrahydrofuranylmethyl-DNJ, N-2-R-tetrahydrofuranylmethyl-DNJ, N-(2-(2,2,2-trifluoroethoxy)ethyl-DNJ, N-2-methoxyethyl-DNJ, N-2-ethoxyethyl-DNJ, N-4-trifluoromethylbenzyl-DNJ, N-alpha-cyano-4-trifluoromethylbenzyl-DNJ, N-4-trifluoromethoxybenzyl-DNJ, N-4-n-pentoxybenzyl-DNJ, and N-4-n-butoxybenzyl-DNJ, or Cl-nonyl DNJ, preferably N-butyl-DNJ (CAS Number 72599-27-0, INN miglustat). DNJ or a derivative thereof can be prepared by methods known in the art, for example as described in WO 2006/125141. DNJ or a derivative thereof may be in the form of a salt, such as DNJ hydrochloride (CAS Number 73285-50-4) or N-butyl-DNJ hydrochloride (CAS Number 210110-90-0). In some embodiments, DNJ is duvoglustat (CAS number 19130-96-2), duvoglustat hydrochloride (CAS number 73285-50-4), miglustat (CAS number 72599-27-0) or miglustat hydrochloride (CAS number 210110-90-0).

The term “ABX” according to the invention means ambroxol (CAS Number 18683-91-5). The term “ABX derivative” means a compound derived from ABX. A ABX derivative may be bromhexine (CAS Number 3572-43-8). ABX or a derivative thereof can be in the form of a salt, such as ABX hydrochloride (CAS Number 23828-92-4). ABX or a derivative thereof can be prepared by methods known in the art, for example as described in US2004/0242700.

In one embodiment, the pharmacological chaperones are duvoglustat (CAS Number 19130-96-2) or duvoglustat hydrochloride (CAS Number 73285-50-4); and ambroxol hydrochloride (CAS Number 23828-92-4). In another embodiment, the pharmacological chaperones are miglustat (CAS Number 72599-27-0) or miglustat hydrochloride (CAS Number 210110-90-0); and ambroxol hydrochloride (CAS Number 23828-92-4).

The term “NAC” according to the invention means N-acetylcysteine (CAS Number 616-91-1 (L)). The term “NAC derivative” means a compound derived from NAC. A NAC derivative may be obtained by coupling NAC with an amino acid by forming ester, amide, and/or hybrid bond(s) between the amino acid and NAC. Any amino acid or amino acid analogs may be used.

The term “pharmaceutically acceptable” means approved by a regulatory agency of the Federal or a state government or listed in the U.S. or European Pharmacopeia or other generally recognized pharmacopeia for use in animals, and humans. A “pharmaceutical composition” means a composition comprising pharmaceutically acceptable carrier. For example, a carrier can be a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like. When the pharmaceutical composition is adapted for oral administration, the tablets or capsules can be prepared by conventional means with pharmaceutically acceptable excipients such as binding agents (e.g. pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or another suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. The composition according to the invention is preferably a pharmaceutical composition.

The term “polypeptide” refers to an amino acid sequence, i.e. a chain of amino acids linked by peptide bonds. The amino acid sequence of the therapeutic GAA polypeptide or its coding sequence can be derived from any source, including avian and mammalian species. The term “avian” as used herein includes, but is not limited to, chickens, ducks, geese, quail, turkeys and pheasants. The term “mammal” or “mammalian” as used herein includes, but is not limited to, humans, simians and other non-human primates, bovines, ovines, caprines, equines, felines, canines, lagomorphs, etc. In embodiments of the invention, the therapeutic GAA polypeptide is a human, mouse or quail, in particular a human, therapeutic GAA polypeptide.

The term “acid α-glucosidase” or “GAA” means an exo-1,4-α-D-glucosidase that hydrolyses both α-1,4 and α-1,6 linkages of oligosaccharides to liberate glucose. A deficiency in GAA results in glycogen storage disease type II (GSDII), also referred to as Pompe disease (although this term formally refers to the infantile onset form of the disease). GAA catalyzes the complete degradation of glycogen with slowing at branching points. The 28 kb human acid α-glucosidase gene on chromosome 17 encodes a 3.6 kb mRNA which produces a 951 amino acid polypeptide [10] and [11]. The enzyme receives co-translational N-linked glycosylation in the endoplasmic reticulum. It is synthesized as a 110-kDa precursor form, which matures by extensive glycosylation modification, phosphorylation and by proteolytic processing through an approximately 90-kDa endosomal intermediate into the final lysosomal 76 and 67 kDa forms [10], [12]; [13] and [14].

The term “glycogen storage disease” or “GSD” refers to a metabolic disorder caused by enzyme deficiencies affecting glycogen synthesis, glycogen breakdown or glycolysis. In a particular, the glycogen storage disease may be one or more type(s) of GSD disclosed in Table 1. According to the invention, the GSD is preferably GSDI (von Gierke's disease), GSDII (Pompe disease), GSDIII (Cori disease), GSDIV, GSDV, GSDVI, GSDVII, GSDVIII or lethal congenital glycogen storage disease of the heart. More particularly, the glycogen storage disease is selected in the group consisting of GSDI, GSDII and GSDIII, even more particularly in the group consisting of GSDII and GSDIII. In an even more particular embodiment, the glycogen storage disease is GSDII.

The term “GAA polypeptide”, without additional precision, refers indistinctly to a GAA having a signal peptide (i.e. GAA precursor) and to a GAA without a signal peptide. It may be a wild type GAA polypeptide or a variant GAA polypeptide.

The term “wild-type GAA polypeptide” refers to the natural form of GAA, for example SEQ ID NO: 2 (corresponding to GenBank Accession number NP_000143.2) or SEQ ID NO: 30 is a wild-type human GAA polypeptide (also found in the Uniprot entry of GAA-accession number P10253; corresponding to GenBank CAA68763.1; SEQ ID NO: 30). Within SEQ ID NO: 2 and SEQ ID NO: 30, amino acid residues 1-27 correspond to the signal peptide of the wild type GAA polypeptide. The term “wild-type GAA polypeptide”, without additional precision, refers indistinctly to a GAA having a signal peptide (i.e. GAA precursor) and to a GAA without a signal peptide.

The term “variant GAA polypeptide” refers to a GAA polypeptide having at least one amino acid modification compared to a wild-type GAA polypeptide, such as substitution, deletion or addition. Illustrative variant GAA polypeptides include SEQ ID NO: 29 (GenBank AAA52506.1); SEQ ID NO: 31 (GenBank: EAW89583.1) and SEQ ID NO: 32 (GenBank ABI53718.1).

Other useful variant GAA polypeptides include those described in Hoefsloot et al. [10]; Van Hove et al. [15], and GenBank Accession number NM_008064 (mouse). Other variant GAA polypeptides include those described in WO2012/145644, WO00/34451 and U.S. Pat. No. 6,858,425. Other useful variant GAA polypeptides also include those described in literature such as GAA II as described by Kunita et al. [16]; GAA polymorphisms and SNPs are described by Hirschhorn, R. and Reuser [1].

The term “therapeutic GAA polypeptide” refers to a GAA polypeptide that can be used in the treatment of GSD. In particular, a therapeutic GAA polypeptide according to the present invention has the functionality of wild-type GAA polypeptide. The functionality of wild-type GAA is to hydrolyse both α-1,4 and α-1,6 linkages of oligosaccharides and polysaccharides, more particularly of glycogen, to liberate glucose. The therapeutic GAA polypeptide may have a hydrolysing activity on glycogen of at least 50%, 60%, 70%, 80%, 90%, 95%, 99%, or at least 100% as compared to the wild-type GAA polypeptide of SEQ ID NO: 2, 30, 29, 31, or SEQ ID NO: 32. The activity of the therapeutic GAA polypeptide may even be of more than 100%, such as of more than 110%, 120%, 130%, 140%, or even more than 150% of the activity of the wild-type GAA protein of SEQ ID NO: 2, 29, 30, 31 or 32. The therapeutic GAA polypeptide may be purified from a recombinant cellular expression system (e.g., mammalian cells or insect cells (see U.S. Pat. Nos. 5,580,757, 6,395,884, 6,458,574, 6,461,609, 210,666, 6,083,725, 6,451,600, 5,236,838, and 5,879,680), human placenta, or animal milk (see U.S. Pat. No. 6,188,045). A therapeutic GAA polypeptide currently approved for the treatment of Pompe disease is the recombinant GAA polypeptide named alglucosidase alfa (marketed by Genzyme, Inc. under the trademark Lumizyme® or Myozyme®).

The therapeutic GAA polypeptide according to the invention comprises a GAA polypeptide moiety and eventually a signal peptide moiety fused to the N-terminal of the GAA polypeptide moiety. Thus, the therapeutic GAA polypeptide may (i) comprise a GAA polypeptide moiety and a signal peptide moiety fused to the N-terminal of the GAA polypeptide moiety or (ii) comprise a GAA polypeptide moiety without a signal peptide moiety fused to the N-terminal of the GAA polypeptide moiety. In some embodiments, the therapeutic GAA polypeptide may also comprise an additional sequence for improving biodistribution, stability and/or tissue uptake of said therapeutic GAA polypeptide.

The term “GAA polypeptide moiety” refers to a GAA polypeptide devoid of a signal peptide. It may be a wild-type GAA polypeptide moiety or a variant GAA polypeptide moiety.

The term “wild-type GAA polypeptide moiety” refers to the natural form of GAA devoid of a signal peptide, for example SEQ ID NO: 1 and SEQ ID NO: 33 are both a wild-type human GAA polypeptide moiety.

The term “variant GAA polypeptide moiety” refers to a GAA polypeptide moiety having at least one amino acid modification compared to a wild-type GAA polypeptide moiety, such as substitution, deletion or addition. Any variant GAA polypeptide may be used as a basis for defining a variant GAA polypeptide moiety. In some embodiments, the variant GAA polypeptide moiety is a truncated GAA polypeptide.

In the context of the present invention, a “truncated GAA polypeptide” means a GAA polypeptide that comprises one or more consecutive amino acids truncated from the N-terminal end of a parent GAA polypeptide devoid of a signal peptide. In one embodiment, the parent GAA polypeptide devoid of a signal peptide is a wild-type GAA polypeptide devoid of a signal peptide, for example a wild-type human GAA polypeptide devoid of a signal peptide represented in SEQ ID NO: 1 or SEQ ID NO: 33. In another embodiment, a parent GAA polypeptide is a variant GAA polypeptide devoid of a signal peptide. Any variant GAA polypeptide known in the art may be used as a basis for defining a parent GAA polypeptide devoid of a signal peptide. Illustrative variant GAA polypeptides include; SEQ ID NO: 29 (GenBank AAA52506.1); SEQ ID NO: 31 (GenBank: EAW89583.1) and SEQ ID NO: 32 (GenBank ABI53718.1). Other useful variants include those described in Hoefsloot et al. [10], Van Hove et al. [15] and GenBank Accession number NM_008064 (mouse). Other variant GAA polypeptides include those described in WO2012/145644, WO00/34451 and U.S. Pat. No. 6,858,425. In a particular embodiment, the parent GAA polypeptide devoid of a signal peptide is derived from the amino acid sequence shown in SEQ ID NO: 2 or SEQ ID NO: 30. Even more particularly, the parent GAA polypeptide devoid of a signal peptide is SEQ ID NO: 1 or SEQ ID: 33, preferably SEQ ID NO: 1.

Especially, the truncated GAA polypeptide according to the invention has 1 to 75 consecutive amino acids truncated at its N-terminal end as compared to a parent GAA polypeptide devoid of its signal peptide. Specifically, the truncated GAA polypeptide may have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 or 75 consecutive amino acids truncated from its N-terminal end as compared to a parent GAA polypeptide devoid of a signal peptide, in particular as compared to SEQ ID NO: 1 or SEQ ID NO: 33. In a preferred embodiment, the truncated GAA polypeptide has 6, 7, 8, 9, 10, 40, 41, 42, 43, 44, 45, 46 or 47 consecutive amino acids truncated at its N-terminal end as compared to a parent GAA polypeptide devoid of its signal peptide, even more particularly 8, 42 or 43 consecutive amino acids truncated at its N-terminal end as compared to a parent GAA polypeptide devoid of a signal peptide, in particular as compared to SEQ ID NO: 1 or SEQ ID NO: 33. In a particular embodiment, the truncated GAA polypeptide of the invention has the sequence shown in SEQ ID NO: 27, SEQ ID NO: 28, SEQ ID NO: 34, SEQ ID NO: 35 and SEQ ID NO: 36. SEQ ID NO: 27 corresponds to a truncated GAA polypeptide having 8 consecutive amino acids truncated from its

N-terminal end as compared to SEQ ID NO: 1. SEQ ID NO: 28 corresponds to a truncated GAA polypeptide having 42 consecutive amino acids truncated from its N-terminal end as compared to SEQ ID NO: 1. SEQ ID NO: 34 corresponds to a truncated GAA polypeptide having 29 consecutive amino acids truncated from its N-terminal end as compared to SEQ ID NO: 1. SEQ ID NO: 35 corresponds to a truncated GAA polypeptide having 43 consecutive amino acids truncated from its N-terminal end as compared to SEQ ID NO: 1. SEQ ID NO: 36 corresponds to a truncated GAA polypeptide having 47 consecutive amino acids truncated from its N-terminal end as compared to SEQ ID NO: 1.

The term “signal peptide moiety” according to the invention means an endogenous (or natural) signal peptide of a wild-type GAA polypeptide, such as the signal peptide encoded by the nucleic acid sequence SEQ ID NO: 4 (named sp1) or an exogenous signal peptide of another protein. Particular exogenous signal peptides workable in the present invention include amino acids 1-20 from chymotrypsinogen B2 (SEQ ID NO: 3) also named sp7), the signal peptide of human alpha-1-antitrypsin (SEQ ID NO: 5, also named sp2), amino acids 1-25 from iduronate-2-sulphatase (SEQ ID NO: 6, also named sp6), and amino acids 1-23 from protease C1 inhibitor (SEQ ID NO: 7, also named sp8). The signal peptides of SEQ ID NO: 3 and SEQ ID NO: 5 to SEQ ID NO: 7, allow higher secretion of the chimeric GAA polypeptide both in vitro and in vivo when compared to a GAA polypeptide comprising its natural signal peptide. In a particular embodiment, the signal peptide has the sequence shown in SEQ ID NO: 3 to 7, or is a functional derivative thereof, i.e. a sequence comprising from 1 to 5, in particular from 1 to 4, in particular from 1 to 3, more particularly from 1 to 2, in particular 1 amino acid deletion(s), insertion(s) or substitution(s) as compared to the sequences shown in SEQ ID NO: 3 to 7, as long as the resulting sequence corresponds to a functional signal peptide, i.e. a signal peptide that allows secretion of a GAA protein. In a particular embodiment, the signal peptide moiety is selected from the group consisting in SEQ ID NO: 3 to 7, preferably SEQ ID NO: 3.

In a specific embodiment, the signal peptide moiety fused to the N-terminal of the GAA polypeptide moiety is selected from SEQ ID NO: 3, SEQ ID NO: 4, SEQ ID NO: 5, SEQ ID NO: 6 or SEQ ID NO: 7, and the GAA polypeptide moiety is selected from SEQ ID NO: 27, SEQ ID NO: 28, SEQ ID NO: 34, SEQ ID NO: 35 or SEQ ID NO: 36.

According to the invention, the term “nucleic acid molecule” refers to a DNA or RNA molecule in single or double stranded form, particularly a DNA. According to the invention, the nucleic acid molecule encodes a therapeutic GAA polypeptide as described above. For example, a nucleic acid molecule encoding a wild-type GAA polypeptide corresponds to SEQ ID NO: 8. A nucleic acid molecule encoding a therapeutic GAA polypeptide devoid of a signal peptide may be the nucleotides sequence 82-2859 of SEQ ID NO: 8, i.e. SEQ ID NO: 9.

The nucleic acid molecule of the invention, encoding a therapeutic GAA polypeptide, can be optimized for expression of the therapeutic GAA polypeptide in vivo. Sequence optimization may include a number of changes in the nucleic acid sequence, including codon optimization, increase of GC content, decrease of the number of CpG islands, decrease of the number of alternative open reading frames (ARFs) and decrease of the number of splice donor and splice acceptor sites. Because of the degeneracy of the genetic code, different nucleic acid molecules may encode the same protein. It is also well known that the genetic codes of different organisms are often biased towards using one of the several codons that encode the same amino acid over the others. Through codon optimization, changes are introduced in a nucleotide sequence that take advantage of the codon bias existing in a given cellular context so that the resulting codon optimized nucleotide sequence is more likely to be expressed in such given cellular context at a relatively high level compared to the non-codon optimised sequence. In a preferred embodiment of the invention, such sequence optimized nucleotide sequence encodes a truncated GAA polypeptide and is codon-optimized to improve its expression in human cells compared to non-codon optimized nucleotide sequences coding for the same truncated GAA polypeptide, for example by taking advantage of the human specific codon usage bias. In a particular embodiment, the nucleic acid molecule encoding a therapeutic GAA polypeptide is codon optimized, and/or has an increased GC content and/or has a decreased number of alternative open reading frames, and/or has a decreased number of splice donor and/or splice acceptor sites, as compared to nucleotides 82-2859 of the wild-type human GAA polypeptide coding sequence, e.g. 82-2859 of SEQ ID NO:8. SEQ ID NO: 8 is a non-optimized nucleotide sequence encoding wild type human GAA polypeptide with signal peptide. SEQ ID NO: 9 is a non-optimized nucleotide sequence encoding wild type human GAA polypeptide devoid of a signal peptide. An optimized nucleotide sequence encoding wild type human GAA polypeptide may be SEQ ID NO: 10 (hGAA co1) or SEQ ID NO: 11 (hGAA co2).

In a particular embodiment, a nucleic acid molecule according to the invention comprises the sequence shown in SEQ ID NO: 12 or SEQ ID NO: 13, encoding the polypeptide having the amino acid sequence shown in SEQ ID NO: 27; the sequence shown in SEQ ID NO: 48 or SEQ ID NO: 49, encoding the polypeptide having the amino acid sequence shown in SEQ ID NO: 28; the sequence shown in SEQ ID NO: 50 or SEQ ID NO: 51, encoding the polypeptide having the amino acid sequence shown in SEQ ID NO: 35; or the sequence shown in SEQ ID NO: 52 or SEQ ID NO: 53, encoding the polypeptide having the amino acid sequence shown in SEQ ID NO: 36. In a preferred embodiment, the nucleic acid molecule of the invention comprises the sequence shown in SEQ ID NO: 12 or SEQ ID NO: 13, encoding the polypeptide having the amino acid sequence shown in SEQ ID NO: 27.

In a preferred embodiment, a nucleic acid molecule encoding a therapeutic GAA polypeptide comprising a GAA polypeptide moiety and a signal peptide moiety fused to the N-terminal of the GAA polypeptide moiety according to the invention is:
SEQ ID NO: 22, including (i) a non-optimized nucleotide sequence encoding a truncated GAA polypeptide having 8 consecutive amino acids truncated from its N-terminal end as compared to the parent hGAA of SEQ ID NO: 1 and (ii) a nucleotide sequence encoding a signal peptide of SEQ ID NO: 5;
SEQ ID NO: 23, including (i) an optimized nucleotide sequence encoding a truncated GAA polypeptide having 8 consecutive amino acids truncated from its N-terminal end as compared to the parent hGAA of SEQ ID NO: 1 (nucleotide sequence derived from the optimized nucleotide sequence of SEQ ID NO: 12) and (ii) a nucleotide sequence encoding a signal peptide of SEQ ID NO: 5;
SEQ ID NO: 24, including (i) an optimized nucleotide sequence encoding a truncated GAA polypeptide having 8 consecutive amino acids truncated from its N-terminal end as compared to the parent hGAA of SEQ ID NO: 1 (nucleotide sequence derived from the optimized nucleotide sequence of SEQ ID NO: 13) and (ii) a nucleotide sequence encoding a signal peptide of SEQ ID NO: 5;
SEQ ID NO: 25, including SEQ ID NO: 12 (nucleotide sequence encoding a truncated GAA polypeptide having 8 consecutive amino acids truncated at its N terminal end as compared to a parent GAA polypeptide moiety of SEQ ID NO: 1) and SEQ ID NO: 54 (nucleotide sequence encoding sp7);
SEQ ID NO: 26, including SEQ ID NO: 48 (nucleotide sequence encoding a truncated GAA polypeptide having 42 consecutive amino acids truncated at its N terminal end as compared to a parent GAA polypeptide moiety of SEQ ID NO: 1) and SEQ ID NO: 54 (nucleotide sequence encoding sp7)
SEQ ID NO: 37, including (i) a non-optimized nucleotide sequence encoding a truncated GAA polypeptide having 29 consecutive amino acids truncated from its N-terminal end as compared to the parent hGAA of SEQ ID NO: 1 (nucleotide sequence derived from the non-optimized nucleotide sequence of SEQ ID NO: 9) and (ii) a nucleotide sequence encoding a signal peptide of SEQ ID NO: 3;
SEQ ID NO: 38, including (i) an optimized nucleotide sequence encoding a truncated GAA polypeptide having 29 consecutive amino acids truncated from its N-terminal end as compared to the parent hGAA of SEQ ID NO: 1 (nucleotide sequence derived from the optimized nucleotide sequence of SEQ ID NO: 12) and a signal peptide of SEQ ID NO: 3;
SEQ ID NO: 39, including (i) an optimized nucleotide sequence encoding a truncated GAA polypeptide having 29 consecutive amino acids truncated from its N-terminal end as compared to the parent hGAA of SEQ ID NO: 1 (nucleotide sequence derived from the optimized nucleotide sequence of SEQ ID NO: 13) and (ii) a nucleotide sequence encoding a signal peptide of SEQ ID NO: 3;
SEQ ID NO: 40: including (i) a non-optimized nucleotide sequence encoding a truncated GAA polypeptide having 42 consecutive amino acids truncated from its N-terminal end as compared to the parent hGAA of SEQ ID NO: 1 (nucleotide sequence derived from the non-optimized nucleotide sequence of SEQ ID NO: 9) and (ii) a nucleotide sequence encoding a signal peptide of SEQ ID NO: 3;
SEQ ID NO: 41, including (i) an optimized nucleotide sequence encoding a truncated GAA polypeptide having 42 consecutive amino acids truncated from its N-terminal end as compared to the parent hGAA of SEQ ID NO: 1 (nucleotide sequence derived from the optimized nucleotide sequence of SEQ ID NO: 13) and (ii) a nucleotide sequence encoding a signal peptide of SEQ ID NO: 3;
SEQ ID NO: 42, including (i) a non-optimized nucleotide sequence encoding a truncated GAA polypeptide having 43 consecutive amino acids truncated from its N-terminal end as compared to the parent hGAA of SEQ ID NO: 1 (nucleotide sequence derived from the non-optimized nucleotide sequence of SEQ ID NO: 9) and (ii) a nucleotide sequence encoding a signal peptide of SEQ ID NO: 3;
SEQ ID NO: 43, including (i) an optimized nucleotide sequence encoding a truncated GAA polypeptide having 43 consecutive amino acids truncated from its N-terminal end as compared to the parent hGAA of SEQ ID NO: 1 (nucleotide sequence derived from the optimized nucleotide sequence of SEQ ID NO: 12) and (ii) a nucleotide sequence encoding a signal peptide of SEQ ID NO: 3; and
SEQ ID NO: 44, including (i) an optimized nucleotide sequence encoding a truncated GAA polypeptide having 43 consecutive amino acids truncated from its N-terminal end as compared to the parent hGAA of SEQ ID NO: 1 (nucleotide sequence derived from the optimized nucleotide sequence of SEQ ID NO: 13) and (ii) a nucleotide sequence encoding a signal peptide of SEQ ID NO: 3;
SEQ ID NO: 45, including (i) a non-optimized nucleotide sequence encoding a truncated GAA polypeptide having 47 consecutive amino acids truncated from its N-terminal end as compared to the parent hGAA of SEQ ID NO: 1 (nucleotide sequence derived from the non-optimized nucleotide sequence of SEQ ID NO: 9) and (ii) a nucleotide sequence encoding a signal peptide of SEQ ID NO: 3;
SEQ ID NO: 46, including (i) an optimized nucleotide sequence encoding a truncated GAA polypeptide having 47 consecutive amino acids truncated from its N-terminal end as compared to the parent hGAA of SEQ ID NO: 1 (nucleotide sequence derived from the optimized nucleotide sequence of SEQ ID NO: 12) and (ii) a nucleotide sequence encoding a signal peptide of SEQ ID NO: 3; and
SEQ ID NO: 47, including (i) an optimized nucleotide sequence encoding a truncated GAA polypeptide having 47 consecutive amino acids truncated from its N-terminal end as compared to the parent hGAA of SEQ ID NO: 1 (nucleotide sequence derived from the optimized nucleotide sequence of SEQ ID NO: 13) and (ii) a nucleotide sequence encoding a signal peptide of SEQ ID NO: 3.

In another preferred embodiment, a nucleic acid molecule encoding a therapeutic GAA polypeptide comprising a GAA polypeptide moiety and a signal peptide moiety fused to the N-terminal of the GAA polypeptide moiety according to the invention is:
(i) a non-optimized nucleotide sequence encoding a truncated GAA polypeptide having 8 consecutive amino acids truncated from its N-terminal end as compared to the parent hGAA of SEQ ID NO: 1 or as compared to the parent hGAA of SEQ ID NO: 33 and (ii) a nucleotide sequence encoding a signal peptide of SEQ ID NO: 4, 5, 6 or 7;
(i) a non-optimized nucleotide sequence encoding a truncated GAA polypeptide having 29 consecutive amino acids truncated from its N-terminal end as compared to the parent hGAA of SEQ ID NO: 1 or as compared to the parent hGAA of SEQ ID NO: 33 and (ii) a nucleotide sequence encoding a signal peptide of SEQ ID NO: 4, 5, 6 or 7;
(i) a non-optimized nucleotide sequence encoding a truncated GAA polypeptide having 42 consecutive amino acids truncated from its N-terminal end as compared to the parent hGAA of SEQ ID NO: 1 or as compared to the parent hGAA of SEQ ID NO: 33 and (ii) a nucleotide sequence encoding a signal peptide of SEQ ID NO: 4, 5, 6 or 7;
(i) a non-optimized nucleotide sequence encoding a truncated GAA polypeptide having 43 consecutive amino acids truncated from its N-terminal end as compared to the parent hGAA of SEQ ID NO: 1 or as compared to the parent hGAA of SEQ ID NO: 33 and (ii) a nucleotide sequence encoding a signal peptide of SEQ ID NO: 4, 5, 6 or 7;
(i) a non-optimized nucleotide sequence encoding a truncated GAA polypeptide having 47 consecutive amino acids truncated from its N-terminal end as compared to the parent hGAA of SEQ ID NO: 1 or as compared to the parent hGAA of SEQ ID NO: 33 and (ii) a nucleotide sequence encoding a signal peptide of SEQ ID NO: 4, 5, 6 or 7;
(i) an optimized nucleotide sequence encoding a truncated GAA polypeptide having 8 consecutive amino acids truncated from its N-terminal end as compared to the parent hGAA of SEQ ID NO: 1 or as compared to the parent hGAA of SEQ ID NO: 33 (nucleotide sequence derived from the optimized sequence of SEQ ID NO: 12) and (ii) a nucleotide sequence encoding a signal peptide of SEQ ID NO: 4, 5, 6 or 7;
(i) an optimized nucleotide sequence encoding a truncated GAA polypeptide having 8 consecutive amino acids truncated from its N-terminal end as compared to the parent hGAA of SEQ ID NO: 1 or as compared to the parent hGAA of SEQ ID NO: 33 (nucleotide sequence derived from the optimized sequence of SEQ ID NO: 13) and (ii) a nucleotide sequence encoding a signal peptide of SEQ ID NO: 4, 5, 6 or 7;
(i) an optimized nucleotide sequence encoding a truncated GAA polypeptide having 29 consecutive amino acids truncated from its N-terminal end as compared to the parent hGAA of SEQ ID NO: 1 or as compared to the parent hGAA of SEQ ID NO: 33 (nucleotide sequence derived from the optimized sequence of SEQ ID NO: 12) and (ii) a nucleotide sequence encoding a signal peptide of SEQ ID NO: 4, 5, 6 or 7;
(i) an optimized nucleotide sequence encoding a truncated GAA polypeptide having 29 consecutive amino acids truncated from its N-terminal end as compared to the parent hGAA of SEQ ID NO: 1 or as compared to the parent hGAA of SEQ ID NO: 33 (nucleotide sequence derived from the optimized sequence of SEQ ID NO: 13) and (ii) a nucleotide sequence encoding a signal peptide of SEQ ID NO: 4, 5, 6 or 7;
(i) an optimized nucleotide sequence encoding a truncated GAA polypeptide having 42 consecutive amino acids truncated from its N-terminal end as compared to the parent hGAA of SEQ ID NO: 1 or as compared to the parent hGAA of SEQ ID NO: 33 (nucleotide sequence derived from the optimized sequence of SEQ ID NO: 12) and (ii) a nucleotide sequence encoding a signal peptide of SEQ ID NO: 4, 5, 6 or 7;
(i) an optimized nucleotide sequence encoding a truncated GAA polypeptide having 42 consecutive amino acids truncated from its N-terminal end as compared to the parent hGAA of SEQ ID NO: 1 or as compared to the parent hGAA of SEQ ID NO: 33 (nucleotide sequence derived from the optimized nucleotide sequence of SEQ ID NO: 13) and (ii) a nucleotide sequence encoding a signal peptide of SEQ ID NO: 4, 5, 6 or 7
(i) an optimized nucleotide sequence encoding a truncated GAA polypeptide having 43 consecutive amino acids truncated from its N-terminal end as compared to the parent hGAA of SEQ ID NO: 1 or as compared to the parent hGAA of SEQ ID NO: 33 (nucleotide sequence derived from the optimized nucleotide sequence of SEQ ID NO: 12) and (ii) a nucleotide sequence encoding a signal peptide of SEQ ID NO: 4, 5, 6 or 7;
(i) an optimized nucleotide sequence encoding a truncated GAA polypeptide having 43 consecutive amino acids truncated from its N-terminal end as compared to the parent hGAA of SEQ ID NO: 1 or as compared to the parent hGAA of SEQ ID NO: 33 (nucleotide sequence derived from the optimized nucleotide sequence of SEQ ID NO: 13) and (ii) a nucleotide sequence encoding a signal peptide of SEQ ID NO: 4, 5, 6 or 7
(i) an optimized nucleotide sequence encoding a truncated GAA polypeptide having 47 consecutive amino acids truncated from its N-terminal end as compared to the parent hGAA of SEQ ID NO: 1 or as compared to the parent hGAA of SEQ ID NO: 33 (nucleotide sequence derived from the optimized nucleotide sequence of SEQ ID NO: 12) and (ii) a nucleotide sequence encoding a signal peptide of SEQ ID NO: 4, 5, 6 or 7;
(i) an optimized nucleotide sequence encoding a truncated GAA polypeptide having 47 consecutive amino acids truncated from its N-terminal end as compared to the parent hGAA of SEQ ID NO: 1 or as compared to the parent hGAA of SEQ ID NO: 33 (nucleotide sequence derived from the optimized nucleotide sequence of SEQ ID NO: 13) and (ii) a nucleotide sequence encoding a signal peptide of SEQ ID NO: 4, 5, 6 or 7.

In a preferred embodiment, the therapeutic GAA polypeptide encoded by a nucleic acid molecule comprises a GAA polypeptide moiety and a signal peptide moiety fused to the N-terminal of the GAA polypeptide moiety, said signal peptide moiety fused to the N-terminal of the GAA polypeptide moiety being selected from SEQ ID NO: 3, SEQ ID NO: 4, SEQ ID NO: 5, SEQ ID NO: 6 or SEQ ID NO: 7, preferably SEQ ID NO: 3, and said GAA polypeptide moiety being selected from SEQ ID NO: 27, SEQ ID NO: 28, SEQ ID NO: 34, SEQ ID NO: 35 or SEQ ID NO: 36, preferably SEQ ID NO: 27.

Most preferably, the nucleic acid molecule is selected from SEQ ID NO: 8, SEQ ID NO: 22, SEQ ID NO: 23, SEQ ID NO: 24, SEQ ID NO: 25, SEQ ID NO: 26, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 or SEQ ID NO: 47, preferably SEQ ID NO: 25.

Preferably, a nucleic acid molecule encoding a therapeutic GAA polypeptide comprising a GAA polypeptide moiety and a signal peptide moiety fused to the N-terminal of the GAA polypeptide moiety according to the invention is:
SEQ ID NO: 25, including SEQ ID NO: 12 (nucleotide sequence encoding a truncated GAA polypeptide having 8 consecutive amino acids truncated at its N terminal end as compared to a parent GAA polypeptide moiety of SEQ ID NO: 1) and SEQ ID NO: 54 (nucleotide sequence encoding sp7), or
SEQ ID NO: 26, including SEQ ID NO: 48 (nucleotide sequence encoding a truncated GAA polypeptide having 42 consecutive amino acids truncated at its N terminal end as compared to a parent GAA polypeptide moiety of SEQ ID NO: 1) and SEQ ID NO: 54 (nucleotide sequence encoding sp7).

A nucleic acid molecule encoding a therapeutic GAA polypeptide according to the invention can be inserted into a nucleic acid construct for expressing said nucleic acid molecule (i.e. the transgene). The nucleic acid construct may comprise a promoter operably linked to one or more expression control sequences and/or other sequences improving the expression of the nucleic acid molecule and/or sequences enhancing the secretion of the therapeutic GAA polypeptide and/or sequences enhancing the tissue uptake of the therapeutic GAA polypeptide. As used herein, the term “operably linked” refers to a linkage of polynucleotide elements in a functional relationship. A nucleic acid is “operably linked” when it is placed into a functional relationship with another nucleic acid sequence. For instance, a promoter, or another transcription regulatory sequence, is operably linked to a coding sequence if it affects the transcription of the coding sequence. Such expression control sequences are known in the art, such as promoters, enhancers (such as cis-regulatory modules (CRMs)), introns, polyA signals, etc.

In particular, the nucleic acid construct comprises a promoter operably linked to the nucleic acid molecule and optionally an intron. The promoter may be an ubiquitous or tissue-specific promoter, in particular a promoter able to promote expression in cells or tissues in which expression of GAA is desirable such as in cells or tissues in which GAA expression is desirable in GAA-deficient patients. In a particular embodiment, the promoter is a liver-specific promoter such as the alpha-1 antitrypsin promoter (hAAT) (SEQ ID NO: 14), the transthyretin promoter, the albumin promoter, the thyroxine-binding globulin (TBG) promoter, the LSP promoter (comprising a thyroid hormone-binding globulin promoter sequence, two copies of an alpha1-microglobulin/bikunin enhancer sequence, and a leader sequence [17], etc. Other useful liver-specific promoters are known in the art, for example those listed in the Liver Specific Gene Promoter Database compiled the Cold Spring Harbor Laboratory (http://rulai.cshl.edu/LSPD/). A preferred promoter in the context of the invention is the hMAT promoter. In another embodiment, the promoter is a promoter directing expression in one tissue or cell of interest (such as in muscle cells), and in liver cells. For example, to some extent, promoters specific of muscle cells such as the desmin, Spc5-12 and MCK promoters may present some leakage of expression into liver cells, which can be advantageous to induce immune tolerance of the subject to the GAA polypeptide expressed from the nucleic acid of the invention. Other tissue-specific or non-tissue-specific promoters may be useful in the practice of the invention. For example, the nucleic acid construct may include a tissue-specific promoter which is a promoter different from a liver specific promoter. For example the promoter may be muscle-specific, such as the desmin promoter (and a desmin promoter variant such as a desmin promoter including natural or artificial enhancers), the SPc5-12 or the MCK promoter. In another embodiment, the promoter is a promoter specific of other cell lineage, such as the erythropoietin promoter, for the expression of the GAA polypeptide from cells of the erythroid lineage. In another embodiment, the promoter is a ubiquitous promoter. Representative ubiquitous promoters include the cytomegalovirus enhancer/chicken beta actin (CAG) promoter, the cytomegalovirus enhancer/promoter (CMV), the PGK promoter, the SV40 early promoter, etc. In addition, the promoter may also be an endogenous promoter such as the albumin promoter or the GAA promoter. In a particular embodiment, the promoter is associated to an enhancer sequence, such as cis-regulatory modules (CRMs) or an artificial enhancer sequence. For example, the promoter may be associated to an enhancer sequence such as the human ApoE control region (or Human apolipoprotein E/C-I gene locus, hepatic control region HCR-1—Genbank accession No. U32510, shown in SEQ ID NO: 15). In a particular embodiment, an enhancer sequence such as the ApoE sequence is associated to a liver-specific promoter such as those listed above, and in particular such as the hAAT promoter. Other CRMs useful in the practice of the present invention include those described in Rincon et al. [18], Chuah et al. [19] or Nair et al.[20]. In another embodiment, the promoter is a hybrid promoter. For example, a hydrid promoter is constituted of liver-selective enhancer(s) operably linked to a short-size muscle-selective promoter, such as spC5-12 promoter, CK6 promoter, CK8 promoter, Acta 1 promoter. The liver-selective enhancer can be selected from HS-CRM1, HS-CRM2, HS-CRM3, HS-CRM4, HS-CRM5, HS-CRM6, HS-CRM7, HS-CRM8, HS-CRM9, HS-CRM10, HS-CRM11, HS-CRM12, HS-CRM13 and HS-CRM14, and can be repeated. Another example of a hybrid promoter is a combination of the ApoE enhancer, hAAT promoter and the spC5.12 promoter (shown in SEQ ID NO: 55); or a combination of the ApoE enhancer, the hAAT promoter and the Syn promoter; or a combination of the ApoE enhancer and the spC5.12 promoter.

In another particular embodiment, the nucleic acid construct comprises an intron, in particular an intron placed between the promoter and the GAA coding sequence. An intron may be introduced to increase mRNA stability and the production of the protein. In a further embodiment, the nucleic acid construct comprises a human beta globin b2 (or HBB2) intron, a coagulation factor IX (FIX) intron, a SV40 intron or a chicken beta-globin intron. In another further embodiment, the nucleic acid construct of the invention contains a modified intron (in particular a modified HBB2 or FIX intron) designed to decrease the number of, or even totally remove, alternative open reading frames (ARFs) found in said intron. Preferably, ARFs are removed whose length spans over 50 bp and have a stop codon in frame with a start codon. ARFs may be removed by modifying the sequence of the intron. For example, modification may be carried out by way of nucleotide substitution, insertion or deletion, preferably by nucleotide substitution. As an illustration, one or more nucleotides, in particular one nucleotide, in an ATG or GTG start codon present in the sequence of the intron of interest may be replaced resulting in a non-start codon. For example, an ATG or a GTG may be replaced by a CTG, which is not a start codon, within the sequence of the intron of interest.

The classical HBB2 intron used in nucleic acid constructs is shown in SEQ ID NO: 16. For example, this HBB2 intron may be modified by eliminating start codons (ATG and GTG codons) within said intron. In a particular embodiment, the modified HBB2 intron comprised in the construct has the sequence shown in SEQ ID NO: 17. The classical FIX intron used in nucleic acid constructs is derived from the first intron of human FIX and is shown in SEQ ID NO: 18. FIX intron may be modified by eliminating start codons (ATG and GTG codons) within said intron. In a particular embodiment, the modified FIX intron comprised in the construct of the invention has the sequence shown in SEQ ID NO: 19. The classical chicken-beta globin intron used in nucleic acid constructs is shown in SEQ ID NO: 20. Chicken-beta globin intron may be modified by eliminating start codons (ATG and GTG codons) within said intron. In a particular embodiment, the modified chicken-beta globin intron comprised in the construct of the invention has the sequence shown in SEQ ID NO: 21. In a preferred embodiment, the intron used in the nucleic acid construct of the invention is the modified HBB2 intron (SEQ ID NO: 17).

In a particular embodiment, the nucleic acid construct of the invention comprises, in the 5′ to 3′ orientation, a promoter optionally preceded by an enhancer, a nucleic acid molecule encoding a therapeutic GAA polypeptide and a polyadenylation signal (such as the bovine growth hormone polyadenylation signal, the SV40 polyadenylation signal, or another naturally occurring or artificial polyadenylation signal). In a particular embodiment, the nucleic acid construct of the invention comprises, in the 5′ to 3′ orientation, a promoter optionally preceded by an enhancer, (such as the ApoE control region), an intron (in particular an intron as defined above), a nucleic acid molecule encoding a therapeutic GAA polypeptide, and a polyadenylation signal. In a further particular embodiment, the nucleic acid construct of the invention comprises, in the 5′ to 3′ orientation, an enhancer such as the ApoE control region, a promoter, an intron (in particular an intron as defined above), a nucleic acid molecule encoding a therapeutic GAA polypeptide, and a polyadenylation signal. In a further particular embodiment of the invention the nucleic acid construct comprises, in the 5′ to 3′ orientation, an ApoE control region, the hAAT-liver specific promoter, a HBB2 intron (in particular a modified HBB2 intron as defined above), a nucleic acid molecule encoding a therapeutic GAA polypeptide, and the bovine growth hormone polyadenylation signal.

According to the invention, the nucleic acid construct may be inserted into a vector, preferably a viral vector. The term “vector” according to the invention means a vector suitable for protein expression, preferably for use in gene therapy. In one embodiment, the vector is a plasmid vector. In another embodiment, the vector is a nanoparticle containing a nucleic acid molecule of the invention, in particular a messenger RNA encoding the GAA polypeptide of the invention. In another embodiment, the vector is a system based on transposons, allowing integration of the nucleic acid molecule or construct of the invention in the genome of the target cell, such as the hyperactive Sleeping Beauty (SB100X) transposon system [21]. In another embodiment, the vector is a viral vector suitable for gene therapy. The vector may target any cell of interest such as liver tissue or cells, muscle cells, CNS cells (such as brain cells or spinal cord cells), or hematopoietic stem cells such as cells of the erythroid lineage (such as erythrocytes). In this case, the nucleic acid construct of the invention also contains sequences suitable for producing an efficient viral vector, as it is well disclosed in the art. In a preferred embodiment, the nucleic acid construct is inserted in a retroviral vector, such as a lentiviral vector, or an AAV vector, such as a single-stranded or double-stranded self-complementary AAV vector. In a much preferred embodiment of the present invention, the viral vector is an AAV vector, such as an AAV vector suitable for transducing liver tissues or cells, more particularly an AAV-1, -2 and AAV-2 variants (such as the quadruple-mutant capsid optimized AAV-2 comprising an engineered capsid with Y44+500+730F+T491V changes, disclosed in Ling et al. [22]), -3 and AAV-3 variants (such as the AAV3-ST variant comprising an engineered AAV3 capsid with two amino acid changes, S663V+T492V, disclosed in Vercauteren et al. [23], -3B and AAV-3B variants, -4, -5, -6 and AAV-6 variants (such as the AAV6 variant comprising the triply mutated AAV6 capsid Y731F/Y705F/T492V form disclosed in Rosario et al. [24], -7, -8, -9, -10 such as -cy10 and -rh10, -rh74, -dj, Anc80, LK03, AAV2i8, porcine AAV serotypes such as AAVpo4 and AAVpo6, etc., vector or a retroviral vector such as a lentiviral vector and an alpha-retrovirus. As it is known in the art, depending on the specific viral vector considered for use, additional suitable sequences will be introduced in the nucleic acid construct of the invention for obtaining a functional viral vector. Suitable sequences include AAV ITRs for an AAV vector, or LTRs for lentiviral vectors. As such, the invention also relates to a nucleic acid construct as described above, flanked by an ITR or an LTR on each side.

In addition, other non-natural engineered variants and chimeric AAV can also be useful. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus. Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. AAV-based recombinant vectors lacking the Rep protein integrate with low efficacy into the host's genome and are mainly present as stable circular episomes that can persist for years in the target cells. Alternatively to using AAV natural serotypes, artificial AAV serotypes may be used in the context of the present invention, including, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid.

In the context of the present invention, the AAV vector comprises an AAV capsid able to transduce the target cells of interest, in particular hepatocytes. In a further particular embodiment, the AAV vector is a pseudotyped vector, i.e. its genome and capsid are derived from AAVs of different serotypes. For example, the pseudotyped AAV vector may be a vector whose genome is derived from one of the above mentioned AAV serotypes, and whose capsid is derived from another serotype. For example, the genome of the pseudotyped vector may have a capsid derived from the AAV8, AAV9, AAVrh74 or AAV2i8 serotype, and its genome may be derived from and different serotype. In a particular embodiment, the AAV vector has a capsid of the AAV8, AAV9 or AAVrh74 serotype, in particular of the AAV8 or AAV9 serotype, more particularly of the AAV8 serotype.

In a specific embodiment, wherein the vector is for use in delivering the transgene to muscle cells, the AAV vector may be selected, among others, in the group consisting of AAV8, AAV9 and AAVrh74.

In another specific embodiment, wherein the vector is for use in delivering the transgene to liver cells, the AAV vector may be selected, among others, in the group consisting of AAV5, AAV8, AAV9, AAV-LK03, AAV-Anc80 and AAV3B.

In another embodiment, the capsid is a modified capsid. In the context of the present invention, a “modified capsid” may be a chimeric capsid or capsid comprising one or more variant VP capsid proteins derived from one or more wild-type AAV VP capsid proteins.

In a particular embodiment, the AAV vector is a chimeric vector, i.e. its capsid comprises VP capsid proteins derived from at least two different AAV serotypes, or comprises at least one chimeric VP protein combining VP protein regions or domains derived from at least two AAV serotypes. Examples of such chimeric AAV vectors useful to transduce liver cells are described in Shen et al. [25] and in Tenney et al. [26]. For example a chimeric AAV vector can derive from the combination of an AAV8 capsid sequence with a sequence of an AAV serotype different from the AAV8 serotype, such as any of those specifically mentioned above. In another embodiment, the capsid of the AAV vector comprises one or more variant VP capsid proteins such as those described in WO2015013313, in particular the RHM4-1, RHM15-1, RHM15-2, RHM15-3/RHM15-5, RHM15-4 and RHM15-6 capsid variants, which present a high liver tropism.

In another embodiment, the modified capsid can be derived also from capsid modifications inserted by error prone PCR and/or peptide insertion (e.g. as described in Bartel et al. [27], or in Michelfelder et al. [28]. In addition, capsid variants may include single amino acid changes such as tyrosine mutants (e.g. as described in Zhong et al. [29]). Another example is the fusion of Anthopleurin-B to the N-terminus of AAV VP2 capsid protein [30].

In addition, the genome of the AAV vector may either be a single stranded or self-complementary double-stranded genome [31]. Self-complementary double-stranded AAV vectors are generated by deleting the terminal resolution site (trs) from one of the AAV terminal repeats. These modified vectors, whose replicating genome is half the length of the wild type AAV genome, have the tendency to package DNA dimers.

In particular, the AVV vector has an AAV-derived capsid, such as an AAV1, AAV2, variant AAV2, AAV3, variant AAV3, AAV3B, variant AAV3B, AAV4, AAV5, AAV6, variant AAV6, AAV7, AAV8, AAV9, AAV10 such as AAVcy10 and AAVrh10, AAVrh74, AAVdj, AAV-Anc80, AAV-LK03, AAV2i8, and porcine AAV, such as AAVpo4 and AAVpo6 capsid or with a chimeric capsid.

In a preferred embodiment, the AAV vector implemented in the practice of the present invention has a single stranded genome, and further preferably comprises an AAV8, AAV9, AAVrh74 or AAV2i8 capsid, in particular an AAV8, AAV9 or AAVrh74 capsid, such as an AAV8 or AAV9 capsid, more particularly an AAV8 capsid. In a particularly preferred embodiment, the invention relates to an AAV vector comprising, in a single-stranded or double-stranded, self-complementary genome (e.g. a single-stranded genome), the nucleic acid construct of the invention. In one embodiment, the AAV vector comprises an AAV8, AAV9, AAVrh74 or AAV2i8 capsid, in particular an AAV8, AAV9 or AAVrh74 capsid, such as an AAV8 or AAV9 capsid, more particularly an AAV8 capsid.

A therapeutic GAA polypeptide, a nucleic acid molecule encoding a therapeutic GAA polypeptide or a nucleic acid construct for expressing a nucleic acid molecule may be prepared by methods known in the art. For example, WO2018/046774 and WO2018/046775 provide methods for preparing a therapeutic GAA polypeptide, a nucleic acid molecule encoding a GAA polypeptide and a nucleic acid construct for expressing a nucleic acid molecule.

According to the invention, the term “uptake of GAA” or “uptake of therapeutic GAA polypeptide” refers to the absorption of GAA by a cell or a tissue. In one embodiment, the tissue is a muscle, such as heart, triceps, quadriceps and diaphragm. In another embodiment, the tissue is a tissue of the nervous system. The term “tissue of the nervous system” refers to a tissue which contains nervous cells such as for example motor neurons, glial cells. In other words, the nervous system consists of the central nervous system comprising the brain and spinal cord, and the peripheral nervous system comprising the branching peripheral nerves, for example a tissue of the central nervous system according to the present invention is the brain and/or spinal cord. The uptake of GAA can be measured by any means known in the art, such as by Western Blot as described in the examples.

The term “subject”, “patient” or “individual”, as used herein, refers to a human or non-human mammal (such as a rodent (mouse, rat), a feline, a canine, or a primate) affected or likely to be affected with GSD. Preferably, the subject is a human, man or woman.

The term “treating” or “treatment” means reversing, alleviating, inhibiting the progress of, or preventing the disorder or condition to which such term applies, or one or more symptoms of such disorder or condition. In particular, the treatment of the disorder may consist in treating the central nervous system (CNS) dysfunctions in GSD, preferably improving the respiratory neuromuscular function and/or decreasing respiratory impairments in a subject.

Kit of Parts

The invention relates to a kit of parts comprising (i) pharmacological chaperones or a pharmaceutically acceptable salt thereof and (ii) a therapeutic acid-alpha glucosidase (GAA) polypeptide or a nucleic acid molecule encoding a therapeutic GAA polypeptide, wherein said pharmacological chaperones are 1-deoxynojirimycin (DNJ) or a derivative thereof and ambroxol (ABX) or a derivative thereof.

The kit of parts of the invention may be used:
as a medicament;
in the treatment of glycogen storage disease (GSD);
in a method for improving the respiratory neuromuscular function and/or decreasing respiratory impairments;
in a method for treating the central nervous system (CNS) dysfunctions in a GSD; and/or
in a method of increasing the uptake of GAA in a tissue of the nervous system, preferably the central nervous system, most preferably the spinal cord, in particular in GSD. In this embodiment, the method may further stabilize GAA in a proper conformation for transporting said GAA to the lysosome.

The kit of parts may be used in a different way between the use of therapeutic GAA polypeptide (i.e. ERT) and the use of nucleic acid molecule encoding a therapeutic GAA polypeptide (i.e. gene therapy).

ERT Using a Therapeutic GAA Polypeptide

ERT increases the amount of GAA polypeptide by exogenously introducing therapeutic GAA polypeptide by way of infusion, preferably therapeutic GAA polypeptide without a signal peptide. After the infusion, the exogenous therapeutic GAA polypeptide is expected to be taken up by tissues through non-specific or receptor-specific mechanism.

According to the invention, the pharmacological chaperones and the therapeutic GAA polypeptide may be administered simultaneously or separately. The pharmacological chaperones may be administered before, simultaneously and/or after the therapeutic GAA polypeptide, as detailed hereafter.

The pharmacological chaperones increase the effectiveness of therapeutic GAA polypeptide, e.g. by increasing the stability of the therapeutic GAA polypeptide in vivo in GSD patients and/or by increasing the tissue uptake of the therapeutic GAA polypeptide in vivo in GSD patients, and in vitro in a formulation or composition. The pharmacological chaperones are useful for enhancing the treatment efficacy of conventional ERT, such as treatment with alglucosidase alpha.

The pharmacological chaperones may be administered by oral administration, nasal administration, transdermal administration or by parenteral injection such as intravenous infusion, subcutaneous injection or intraperitoneal injection, preferably by oral administration or intravenous infusion, more preferably by oral administration.

The therapeutic GAA polypeptide may be administered by oral administration, nasal administration, transdermal administration or by parenteral injection such as intravenous infusion, subcutaneous injection or intraperitoneal injection, preferably by intravenous infusion or subcutaneous injection. More preferably, the therapeutic GAA polypeptide is administered intravenously in a sterile solution for injection.

In one embodiment, the therapeutic GAA polypeptide and the pharmacological chaperones are formulated separately. In this embodiment, the pharmacological chaperones and the therapeutic GAA polypeptide may be administered according to the same route, e.g., intravenous infusion, or preferably, by different routes, e.g., intravenous infusion for the therapeutic GAA polypeptide, and oral administration for the pharmacological chaperones. The therapeutic GAA polypeptide is administered by any of the routes, but preferably administration is parenteral.

In another embodiment, the pharmacological chaperones and therapeutic GAA polypeptide are formulated in a single composition. Such a composition enhances stability of the therapeutic GAA polypeptide during storage and in vivo administration, thereby reducing costs and increasing therapeutic efficacy. The formulation is preferably suitable for parenteral administration, including intravenous subcutaneous, and intraperitoneal, however, formulations suitable for other routes of administration such as oral, intranasal, or transdermal are also contemplated.

The pharmacological chaperones may be formulated in the same composition or separately, preferably separately. For example, one of the pharmacological chaperone (DNJ or ABX) and the therapeutic GAA polypeptide may be formulated in one composition, and the other chaperone (ABX or DNJ) may be formulated in another composition. In another example, the chaperones (ABX and DNJ) and the therapeutic GAA polypeptide are formulated in three separate compositions.

In some embodiments, DNJ is formulated in a separate composition, e.g. the composition commercially available under the name Zavesca® (corresponding to miglustat (CAS number 72599-27-0)). Pharmaceutical compositions comprising DNJ are described in WO2006/125141 and WO2014/110270.

In some embodiments, ABX is formulated in a separate composition, e.g. the composition commercially available under the name Ambrobene®, Aponova®, Mucoangin®, Ambroxol Mylan®. Pharmaceutical compositions comprising ABX are described in EP1543826.

In some embodiments, the therapeutic GAA polypeptide is formulated in a separate composition, e.g. the composition commercially available under the name Lumizyme® or Myozyme®.

The timing of administration will vary based on several factors including, without limitation, the route of administration, the GSD treated or the subject's age. One skilled in the art can readily determine, based on its knowledge in this field, the timing of administration required based on these factors and others.

When the therapeutic GAA polypeptide and pharmacological chaperones are formulated separately, administration may be simultaneous, or the pharmacological chaperones may be administered prior to, or after the therapeutic GAA polypeptide. For example, where the therapeutic GAA polypeptide is administered intravenously, the pharmacological chaperones may be administered during a period from 0 hours to 6 hours later. Alternatively, the pharmacological chaperones may be administered from 0 to 6 hours prior to the therapeutic GAA polypeptide. In a preferred embodiment, where the pharmacological chaperones and therapeutic GAA polypeptide are administered separately, and where the pharmacological chaperones have a short circulating half-life {e.g., small molecule), the pharmacological chaperones may be orally administered continuously, such as daily, in order to maintain a constant level in the circulation. Such constant level will be one that has been determined to be non-toxic to the patient, and optimal regarding interaction with the therapeutic GAA polypeptide during the time of administration to confer a non-inhibitory, therapeutic effect. In another embodiment, the pharmacological chaperones are administered during the time period required for turnover of the therapeutic GAA polypeptide (which will be extended by administration of the pharmacological chaperones).

The dose of therapeutic GAA polypeptide administered to the subject in need thereof will vary based on several factors including, without limitation, the route of administration, the GSD treated or the subject's age. One skilled in the art can readily determine, based on its knowledge in this field, the dosage range required based on these factors and others. According to current methods, the concentration of therapeutic GAA polypeptide is generally between about 0.05-50.0 mg/kg of body weight, typically administered weekly or biweekly. The therapeutic GAA polypeptide can be administered at a dosage ranging from 0.1 mg/kg to about 30 mg/kg, preferably from about 0.1 mg/kg to about 20 mg/kg. Regularly repeated doses of the protein are necessary over the life of the patient. Subcutaneous injections maintain longer term systemic exposure to the therapeutic GAA polypeptide. The subcutaneous dosage is preferably 0.1-5.0 mg of the therapeutic GAA polypeptide per kg body weight biweekly or weekly. The therapeutic GAA polypeptide is also administered intravenously, e.g., in an intravenous bolus injection, in a slow push intravenous injection, or by continuous intravenous injection. Continuous IV infusion (e.g., over 2-6 hours) allows the maintenance of specific levels in the blood. For example, the therapeutic GAA polypeptide without signal peptide currently approved for the treatment of Pompe disease, named alglucosidase alfa (marketed by Genzyme, Inc. under the trademark Lumizyme® or Myozyme), is administered every 2 weeks by intravenous infusion at a dose of 20 mg per kg body weight.

The dose of pharmacological chaperones administered to the subject in need thereof will vary based on several factors including, without limitation, the route of administration, the GSD treated, the subject's age or the amount of therapeutic GAA polypeptide administered to the subject. One skilled in the art can readily determine, based on its knowledge in this field, the dosage range required based on these factors and others.

The dose of the pharmacological chaperones which will be effective in the treatment of a glycogen storage disease can be determined by standard clinical techniques. In addition, in vivo and/or in vitro assays may optionally be employed to help predict optimal dosage ranges. The precise dose to be employed in the formulation will also depend on the route of administration, and the seriousness of the disease, and should be decided according to the judgment of the practitioner and each patient's circumstances. The dosage of pharmacological chaperones administered to the subject in need thereof will vary based on several factors including, without limitation, the route of administration, the specific disease treated, the subject's age. One skilled in the art can readily determine, based on its knowledge in this field, the dosage range required based on these factors and others.

In case of a treatment comprising administering DNJ or a derivative thereof, preferably DNJ, the typical doses of DNJ or a derivative thereof would be 1 gram (g), 2 g, 3 g, 4 g, 5 g, 6 g or more, during for example, 1 to 10 days, preferably 3 to 7 days. The treatment can be interrupted by 1 to 10 days without treatment, preferably 3 to 7 days. After the days of interruption, the treatment can be administered following the previous typical doses and duration as mentioned above. For example, typical doses are 2.5 g during 3 days and 4 days without treatment, for example 5 g during 3 days and 4 days without treatment, or for example 5 g during 7 days and 7 days without treatment (clinical trial identifier number NCT00688597).

In case of a treatment comprising administering ABX or a derivative thereof, preferably ABX hydrochloride, the typical doses of ABX or a derivative thereof would be 50 to 500 mg, preferably 60 to 420 mg, repeated for example three times per day (clinical trial identifier number NCT02941822).

Gene Therapy Using a Nucleic Acid Molecule Encoding a Therapeutic GAA Polypeptide

The present invention also contemplates the use of pharmacological chaperones in combination with gene therapy in GSD. Such a combination will enhance the efficacy of gene therapy by increasing the level of expression of the therapeutic GAA polypeptide in vivo in GSD patients, by increasing the stability of the expressed therapeutic GAA polypeptide in vivo in GSD patients and/or by increasing the tissue uptake of the expressed therapeutic GAA polypeptide in vivo in GSD patients.

According to the invention, the pharmacological chaperones and the nucleic acid molecule encoding a therapeutic GAA polypeptide may be administered simultaneously or separately. The pharmacological chaperones may be administered before, simultaneously and/or after the nucleic acid molecule encoding a therapeutic GAA polypeptide, as detailed hereafter.

In a preferred embodiment, the pharmacological chaperones are administered after the nucleic acid molecule encoding a therapeutic GAA polypeptide. For example, the pharmacological chaperones can be administered one month, two months or more after the administration of the nucleic acid molecule encoding a therapeutic GAA polypeptide. According to this embodiment, the pharmacological chaperones and the nucleic acid molecule encoding a therapeutic GAA polypeptide are formulated separately.

The nucleic acid molecule delivery into a patient may be either direct, in which case the patient is directly exposed to the nucleic acid molecule, a nucleic acid construct or a vector, e.g. a viral vector, or indirect, in which case, cells are first transformed with the nucleic acid molecule, a nucleic acid construct or a vector, e.g. a viral vector, in vitro, then transplanted into the patient. These two approaches are known, respectively, as in vivo and ex vivo gene therapy. In case of delivery of liver cells, said cells may be cells previously collected from the subject and engineered by introducing therein a nucleic acid molecule or a nucleic acid construct encoding a therapeutic GAA polypeptide to thereby make them able to produce said therapeutic GAA polypeptide.

In some embodiments, the nucleic acid molecule is inserted into a nucleic acid construct for expressing said nucleic acid molecule, said nucleic acid construct being inserted into a viral vector selected from a retroviral vector, such as a lentiviral vector, or an AAV vector, such as a single-stranded or double-stranded self-complementary AAV vector, preferably an AAV vector having an AAV8, AAV9, AAVrh74 or AAV2i8 capsid, in particular an AAV8, AAV9 or AAVrh74 capsid, more particularly an AAV8 capsid.

The administration of the nucleic acid molecule, the nucleic acid construct or the viral vector encoding a therapeutic GAA polypeptide is for example but are not limited to intradermal, intramuscular, intraperitoneal, intravenous, subcutaneous, intranasal, epidural, and oral routes. In a particular embodiment, the administration is via the intravenous or intramuscular route. The nucleic acid molecule encoding a therapeutic GAA polypeptide, whether vectorized or not, may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In a specific embodiment, it may be desirable to administer the nucleic acid molecule, the nucleic acid construct or the viral vector encoding a therapeutic GAA polypeptide locally to the area in need of treatment, e.g. the liver. This may be achieved, for example, by means of an implant, said implant being of a porous, nonporous, or gelatinous material, including membranes, such as sialastic membranes, or fibers.

The amount of the nucleic acid construct, the vector molecule, the nucleic acid construct or the viral vector encoding a therapeutic GAA polypeptide which will be effective in the treatment of a glycogen storage disease can be determined by standard clinical techniques. In addition, in vivo and/or in vitro assays may optionally be employed to help predict optimal dosage ranges. The precise dose to be employed in the formulation will also depend on the route of administration, and the seriousness of the disease, and should be decided according to the judgment of the practitioner and each patient's circumstances. The dosage the nucleic acid construct, the vector molecule, the nucleic acid construct or the viral vector encoding a therapeutic GAA polypeptide administered to the subject in need thereof will vary based on several factors including, without limitation, the route of administration, the specific disease treated, the subject's age or the level of expression necessary to achieve the therapeutic effect. One skilled in the art can readily determine, based on its knowledge in this field, the dosage range required based on these factors and others. In case of a treatment comprising administering a viral vector, such as an AAV vector, to the subject, typical doses of the vector are of at least 1×10⁸ vector genomes per kilogram body weight (vg/kg), such as at least 1×10⁹ vg/kg, at least 1×10¹⁰ vg/kg, at least 1×10¹¹ vg/kg, at least 1×10¹² vg/kg at least 1×10¹³ vg/kg, or at least 1×10¹⁴ vg/kg. Thanks to the pharmacological chaperones, the doses of the nucleic acid construct, the vector molecule, the nucleic acid construct or the viral vector encoding a therapeutic GAA polypeptide according to the invention may be decreased compared to the typical doses.

In an aspect of the invention, the subject receives repeated administration of a nucleic acid molecule encoding a therapeutic GAA polypeptide. In this aspect, said administration may be repeated at least once or more, and may even be considered to be done according to a periodic schedule, such as once per year. The periodic schedule may also comprise an administration once every 2, 3, 4, 5, 6, 7, 8, 9 or 10 year, or more than 10 years. In another particular embodiment, administration of each administration of a viral vector is done using a different virus for each successive administration, thereby avoiding a reduction of efficacy because of a possible immune response against a previously administered viral vector. For example, a first administration of a viral vector comprising an AAV8 capsid may be done, followed by the administration of a vector comprising an AAV9 capsid, or even by the administration of a virus unrelated to AAVs, such as a retroviral or lentiviral vector. Alternatively, transient immunosuppression can be used to avoid immune responses against the capsid.

Methods for gene therapy are known in the art and some embodiments are also detailed above in the definition part.

The pharmacological chaperones may be administered by oral administration, nasal administration, transdermal administration or by parenteral injection such as intravenous infusion, subcutaneous injection, intraperitoneal injection preferably by oral administration or intravenous infusion, more preferably by oral administration.

The pharmacological chaperones may be formulated in the same composition or separately, preferably separately. DNJ may be formulated in a separate pharmaceutical composition, e.g. the composition commercially available under the name Zavesca® (corresponding to miglustat (CAS number 72599-27-0)). Pharmaceutical compositions comprising DNJ are described in WO2006/125141 and WO2014/110270. ABX may be formulated in a separate pharmaceutical composition, e.g. the composition commercially available under the name Ambrobene®, Aponova®, Mucoangin®, Ambroxol Mylan®. Pharmaceutical compositions comprising ABX are described in EP1543826.

The pharmacological chaperones may be orally administered continuously, such as daily, in order to maintain a constant level in the circulation. Such constant level will be one that has been determined to be non-toxic to the patient, and optimal regarding interaction with the expressed therapeutic GAA polypeptide during the time of administration to confer a non-inhibitory, therapeutic effect.

The dose of the pharmacological chaperones that may be effective in the treatment of a glycogen storage disease according to the invention is described above in “ERT using a therapeutic GAA polypeptide”.

In a preferred embodiment, the kit of parts comprises (i) duvoglustat and ambroxol hydrochloride and (ii) a viral vector in which a nucleic acid construct for expressing a nucleic acid molecule encoding a therapeutic GAA polypeptide is inserted.

In an even more preferred embodiment, the kit of parts comprises (i) duvoglustat and ambroxol hydrochloride and (ii) a viral vector in which a nucleic acid construct for expressing a nucleic acid molecule of SEQ ID NO: 25 or SEQ ID NO: 26 is inserted.

Composition

The invention also relates to a composition comprising pharmacological chaperones or a pharmaceutically acceptable salt thereof, for use in the treatment of glycogen storage disease (GSD) in a subject receiving a therapeutic acid-alpha glucosidase (GAA) treatment for treating said GSD, wherein said pharmacological chaperones are 1-deoxynojirimycin (DNJ) or a derivative thereof and ambroxol (ABX) or a derivative thereof.

The invention also relates to a composition comprising pharmacological chaperones or a pharmaceutically acceptable salt thereof, for use in a method of increasing the uptake of GAA in a tissue of the nervous system in a subject receiving a therapeutic GAA treatment for treating a GSD, wherein said pharmacological chaperones are DNJ or a derivative thereof and ABX or a derivative thereof. In some embodiments, the tissue of the nervous system is for example a tissue of the central nervous system, preferably spinal cord. In some embodiments, the method further stabilizes GAA in a proper conformation for transport to the lysosome.

The invention also relates to a composition comprising pharmacological chaperones or a pharmaceutically acceptable salt thereof, for use in a method of improving the respiratory neuromuscular function and/or decreasing respiratory impairments in a subject receiving a therapeutic GAA treatment for treating a GSD, wherein said pharmacological chaperones are DNJ or a derivative thereof and ABX or a derivative thereof.

The invention also relates to a composition comprising pharmacological chaperones or a pharmaceutically acceptable salt thereof, for use in a method for treating the central nervous system (CNS) dysfunctions in GSD in a subject receiving a therapeutic GAA treatment for treating a GSD, wherein said pharmacological chaperones are DNJ or a derivative thereof and ABX or a derivative thereof.

According to the invention, the composition is administered in a subject receiving a therapeutic GAA treatment. The therapeutic GAA treatment may be a therapeutic GAA polypeptide (ERT), preferably a therapeutic GAA polypeptide without signal peptide, or a nucleic acid molecule encoding a therapeutic GAA polypeptide (gene therapy), as detailed above. Preferably, the composition is administered in a subject receiving a nucleic acid molecule encoding a therapeutic GAA polypeptide. In some embodiments, the nucleic acid molecule is inserted into a nucleic acid construct for expressing said nucleic acid molecule, said nucleic acid construct being inserted into a viral vector selected from a retroviral vector, such as a lentiviral vector, or an AAV vector, such as a single-stranded or double-stranded self-complementary AAV vector, preferably an AAV vector having an AAV8, AAV9, AAVrh74 or AAV2i8 capsid, in particular an AAV8, AAV9 or AAVrh74 capsid, more particularly an AAV8 capsid.
The composition of the invention may be administered before, simultaneously and/or after the therapeutic GAA treatment, preferably before, simultaneously and/or after the nucleic acid molecule encoding a therapeutic GAA polypeptide. For example, where the therapeutic GAA polypeptide is administered intravenously, the composition may be administered during a period from 0 hours to 6 hours later. In a preferred embodiment, where the composition of the invention and therapeutic GAA polypeptide are administered separately, and where the pharmacological chaperones have a short circulating half-life {e.g., small molecule), the composition may be orally administered continuously, such as daily, in order to maintain a constant level in the circulation. Such constant level will be one that has been determined to be non-toxic to the patient, and optimal regarding interaction with the therapeutic GAA polypeptide during the time of administration to confer a non-inhibitory, therapeutic effect. In another embodiment, the composition is administered during the time period required for turnover of the therapeutic GAA polypeptide (which will be extended by administration of the pharmacological chaperones). For example, where the nucleic acid molecule encoding a therapeutic GAA polypeptide is administered intravenously or by intramuscular route, the composition of the invention may be administered two months after treatment and may be orally administered continuously, such as daily, in order to maintain a constant level in the circulation.

The composition of the invention may be administered by oral administration, nasal administration, transdermal administration or by parenteral injection such as intravenous infusion, subcutaneous injection, intraperitoneal injection preferably by oral administration or intravenous infusion, more preferably by oral administration.

Each of the pharmacological chaperones of the composition may be formulated separately or in the same composition. For example, one of the pharmacological chaperone DNJ may be formulated in one composition and the other chaperone ABX may be formulated in another composition.

In one embodiment, ABX and DNJ are formulated separately. In this embodiment, the separate compositions may be administered according to the same route or by different routes, preferably to the same route, more preferably by oral administration.

In another embodiment, ABX and DNJ are formulated in the same composition. In this embodiment, the composition is suitable for oral, intranasal, or transdermal administration, preferably for oral administration.

DNJ may be formulated in a separate pharmaceutical composition, e.g. the composition commercially available under the name Zavesca® (corresponding to miglustat (CAS number 72599-27-0)). Pharmaceutical compositions comprising DNJ are described in WO2006/125141 and WO2014/110270.

ABX may be formulated in a separate pharmaceutical composition, e.g. the composition commercially available under the name Ambrobene®, Aponova®, Mucoangin®, Ambroxol Mylan®. Pharmaceutical compositions comprising ABX are described in EP1543826.

The dose of pharmacological chaperones in the composition administered to the subject in need thereof will vary based on several factors including, without limitation, the route of administration, the GSD treated, the subject's age or the therapeutic GAA treatment (e.g. ERT or gene therapy). One skilled in the art can readily determine, based on its knowledge in this field, the dosage range required based on these factors and others. For example, the composition of the invention can comprise 1 g, 2 g, 3 g, 5 g, 6 g or more of DNJ or a derivative thereof, and 50 to 500 mg, for example 60 to 420 mg of ABX or a derivative thereof.

In a preferred embodiment, the composition comprises duvoglustat and ambroxol hydrochloride for use in the treatment of GSD in a subject receiving a therapeutic GAA treatment, in which the therapeutic GAA treatment is a viral vector in which a nucleic acid construct for expressing a nucleic acid molecule encoding a therapeutic GAA polypeptide is inserted, for treating said GSD.

In an even preferred embodiment, the composition comprises duvoglustat and ambroxol hydrochloride for use in the treatment of GSDII in a subject receiving a viral vector in which a nucleic acid construct for expressing a nucleic acid molecule of SEQ ID NO: 25 or SEQ ID NO: 26 is inserted, for treating said GSDII.

Method of Treatment

The invention also relates to a method for treating glycogen storage disease (GSD) in a subject receiving therapeutic GAA treatment for treating said GSD, comprising administering to the subject a composition comprising pharmacological chaperones, wherein the pharmacological chaperones are DNJ or a derivative thereof and ABX or a derivative thereof.

The invention also relates to a method for increasing the uptake of GAA in a tissue of the nervous system, in a subject receiving a therapeutic GAA treatment for treating a GSD, comprising administering to the subject a composition comprising pharmacological chaperones, wherein the pharmacological chaperones are DNJ or a derivative thereof and ABX or a derivative thereof.

The invention also relates to method for improving the respiratory neuromuscular function and/or decreasing respiratory impairments in a subject receiving a therapeutic GAA treatment for treating a GSD, comprising administering to the subject a composition comprising pharmacological chaperones, wherein the pharmacological chaperones are DNJ or a derivative thereof and ABX or a derivative thereof.

The invention also relates to a method for treating the central nervous system (CNS) dysfunctions of GSD in a subject receiving a therapeutic GAA treatment for treating a GSD, comprising administering to the subject a composition comprising pharmacological chaperones, wherein the pharmacological chaperones are DNJ or a derivative thereof and ABX or a derivative thereof.

Definitions and embodiments regarding administration of the pharmacological chaperones and the therapeutic GAA treatment are described here above, in particular in the “kit of parts” section.

Other Objects

The inventors have also shown that the pharmacological chaperones DNJ, ABX or NAC increase the uptake of GAA in tissue, in particular in GSD.

DNJ and a Nucleic Acid Molecule Encoding a Therapeutic GAA Polypeptide

The invention relates to kit of parts, comprising (i) a pharmacological chaperone or a pharmaceutically acceptable salt thereof and (ii) a nucleic acid molecule encoding a therapeutic GAA polypeptide, wherein said pharmacological chaperone is 1-deoxynojirimycin (DNJ) or a derivative thereof. Said kit of parts may be used:
as a medicament.
in the treatment of glycogen storage disease (GSD); and/or
in a method of increasing the uptake of GAA in a tissue of the nervous system, in particular in GSD.

The invention also relates to a composition comprising a pharmacological chaperone or a pharmaceutically acceptable salt thereof, for use in the treatment of glycogen storage disease (GSD) in a subject receiving a nucleic acid molecule encoding a therapeutic GAA polypeptide for treating said GSD, wherein said pharmacological chaperone is 1-deoxynojirimycin (DNJ) or a derivative thereof.

The invention also relates to a composition comprising a pharmacological chaperone or a pharmaceutically acceptable salt thereof, for use in a method of increasing the uptake of GAA in a tissue of the nervous system in a subject receiving a nucleic acid molecule encoding a therapeutic GAA polypeptide for treating a GSD, wherein said pharmacological chaperone is 1-deoxynojirimycin (DNJ) or a derivative thereof.

The invention also relates to a composition comprising a pharmacological chaperone or a pharmaceutically acceptable salt thereof, for use in a method of increasing the uptake of GAA in a tissue of the nervous system, preferably in the central nervous system, more preferably in spinal cord, in a subject receiving a nucleic acid molecule encoding a therapeutic GAA polypeptide for treating a GSD, wherein said pharmacological chaperone is 1-deoxynojirimycin (DNJ) or a derivative thereof.

The invention also relates to a composition comprising a pharmacological chaperone or a pharmaceutically acceptable salt thereof, for use in a method for treating the central nervous system (CNS) dysfunctions of GSD in a subject receiving a nucleic acid molecule encoding a therapeutic GAA polypeptide for treating a GSD, wherein said pharmacological chaperone is 1-deoxynojirimycin (DNJ) or a derivative thereof.

The invention also relates to a composition comprising a pharmacological chaperone or a pharmaceutically acceptable salt thereof, for use in a method of improving the respiratory neuromuscular function and/or decreasing respiratory impairments in a subject receiving a nucleic acid molecule encoding a therapeutic GAA polypeptide for treating a GSD, wherein said pharmacological chaperone is 1-deoxynojirimycin (DNJ) or a derivative thereof.

The invention also relates to a method for treating glycogen storage disease (GSD) in a subject receiving a nucleic acid molecule encoding a therapeutic GAA polypeptide for treating said GSD, comprising administering to the subject a composition comprising a pharmacological chaperone wherein the pharmacological chaperone is DNJ or a derivative thereof.

The invention also relates to a method for increasing the uptake of GAA in a tissue of the nervous system, in a subject receiving a nucleic acid molecule encoding a therapeutic GAA polypeptide for treating a GSD, comprising administering to the subject a composition comprising a pharmacological chaperone wherein the pharmacological chaperone is DNJ or a derivative thereof.

The invention also relates to a method for improving the respiratory neuromuscular function and/or decreasing respiratory impairments in a subject receiving a nucleic acid molecule encoding a therapeutic GAA polypeptide for treating a GSD, comprising administering to the subject a composition comprising a pharmacological chaperone wherein the pharmacological chaperone is DNJ or a derivative thereof.

The invention also relates to a method for treating the central nervous system (CNS) dysfunctions of GSD in a subject receiving a nucleic acid molecule encoding a therapeutic GAA polypeptide for treating a GSD, comprising administering to the subject a composition comprising a pharmacological chaperone wherein the pharmacological chaperone is DNJ or a derivative thereof.

NAC and a Nucleic Acid Molecule Encoding a Therapeutic GAA Polypeptide

The invention also relates to a kit of parts comprising (i) a pharmacological chaperone or a pharmaceutically acceptable salt thereof and (ii) a nucleic acid molecule encoding a therapeutic GAA polypeptide, wherein said pharmacological chaperone is N-acetylcysteine (NAC) or a derivative thereof. Said kit of parts may be used as a medicament, in particular in the treatment of glycogen storage disease (GSD).

The invention also relates to composition comprising a pharmacological chaperone or a pharmaceutically acceptable salt thereof, for use in the treatment of glycogen storage disease (GSD) in a subject receiving a nucleic acid molecule encoding a therapeutic GAA polypeptide for treating said GSD, wherein the pharmacological chaperone is N-acetylcysteine (NAC) or a derivative thereof.

The invention also relates to a method for treating glycogen storage disease (GSD) in a subject receiving a nucleic acid molecule encoding a therapeutic GAA polypeptide for treating said GSD, comprising administering to the subject a composition comprising a pharmacological chaperone wherein the pharmacological chaperone is NAC or a derivative thereof.

ABX and a Therapeutic GAA Polypeptide or a Nucleic Acid Molecule Encoding a Therapeutic GAA Polypeptide

The invention also relates to a kit of parts comprising (i) a pharmacological chaperone or a pharmaceutically acceptable salt thereof and (ii) a therapeutic GAA polypeptide or a nucleic acid molecule encoding a therapeutic GAA polypeptide, wherein the pharmacological chaperone is ambroxol (ABX) or a derivative thereof. Said kit of parts may be used as a medicament, in particular in the treatment of glycogen storage disease (GSD).

The invention also relates to composition comprising pharmacological chaperone or a pharmaceutically acceptable salt thereof, for use in the treatment of glycogen storage disease (GSD) in a subject receiving a therapeutic acid-alpha glucosidase (GAA) treatment for treating said GSD, wherein said pharmacological chaperone is ambroxol (ABX) or a derivative thereof.

The invention also relates to a method for treating glycogen storage disease (GSD) in a subject receiving therapeutic GAA treatment for treating said GSD, comprising administering to the subject a composition comprising a pharmacological chaperone wherein the pharmacological chaperone is ABX or a derivative thereof.

The invention will be further illustrated by the following figures and examples. However, these examples and figures should not be interpreted in any way as limiting the scope of the invention.

### EXAMPLES

### Example 1: Study on WT Mice

Materials and Methods

Construction of the AAV8-Sp7-Δ8-coGA Vector Expressing Therapeutic Human GAA Polypeptide (hGAA)

AAV Vector Production

AAV8 vectors were produced using an adenovirus-free transient transfection method (Matsushita et al., [32]) and purified as described earlier (Ayuso et al., [33]). Titers of the AAV vector stocks were determined using a quantitative real-time PCR (qPCR) and confirmed by SDS-PAGE followed by SYPRO® Ruby protein gel stain and band densitometry. A nucleic acid construct has been inserted into the AAV vector, especially into the expression cassette sequence comprising the two ITRs and the bGH polyA. Said nucleic acid construct comprises from 5′ to 3′: the ApoE control region SEQ ID NO: 15, the hAAT-liver specific promoter SEQ ID NO: 14, the modified HBB2 intron SEQ ID NO: 17 and the nucleic acid molecule SEQ ID NO: 25. The resulting AAV8 vector is called AAV8-sp7-Δ8-co1GAA vectors.

In Vivo Studies

Standard animal care and housing were in accordance with the national guidelines. Animal experiments were approved by the ethical committee of the CERFE (Approval Number: 2015008D) in accordance with the European Directive 2010/63/EU.

Six-eight-week-old C57Bl/6 male mice were divided into 9 groups of five mice. 5×10¹¹ vg/kg of AAV8-sp7-Δ8-co1GAA vectors were administered at day 0 intravenously in awake, restrained animals via tail vein injection. Two months after the AAV treatment, 7 pharmacological chaperone molecules or combination of them were orally administered to the mice using a “3 on/4 off” regimen (three consecutive days of treatment followed by four consecutive days with drinking water only) for four weeks. At the end of this four-week period, all mice were sacrificed and different organs and muscles were collected (liver, heart, brain, spinal cord, diaphragm, quadriceps, and triceps). C57Bl/6 mice were injected either with PBS (CTRL) or with 5×10¹¹ vg/kg of the AAV8 vector expressing secretable therapeutic hGAA polypeptide (AAV-GAA).

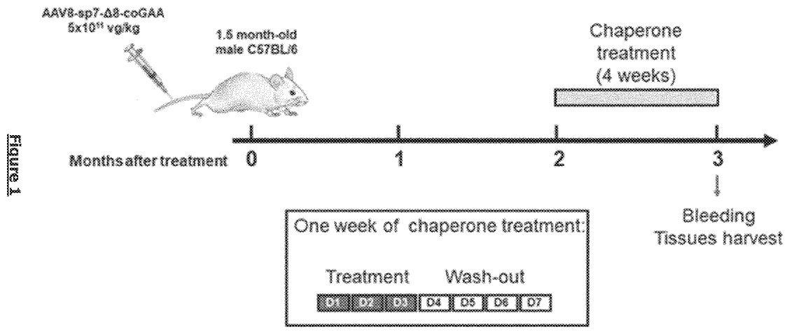

Two months after vector injection, mice were treated for four weeks with 100 mg/kg/die of duvoglustat (DNJ, AX61Q1, Interchim, San Diego, Calif.), 100 mg/kg/die of duvoglustat combined with 25 mg/kg/die of ambroxol (DNJ-ABX), 25 mg/kg/die of ambroxol hydrochloride (ABX, A9797, Sigma, Saint Louis, Mo.), 2 mg/kg/die of voglibose (VOGLIBOSE, S4101, Selleckchem, Houston, Tex.), 20 mg/kg/die of acarbose (ACARBOSE, S1271, Selleckchem, Houston, Tex.), 2 mg/kg/die of miglitol (MIGLITOL, S2589, Selleckchem, Houston, Tex.) or 4200 mg/kg/die of N-acetyl-cysteine (NAC, A7250, Sigma, Saint Louis, Mo.) dissolved in drinking water (FIG. 1). One group received normal drinking water (WATER).

Three months after vector injection, mice were sacrificed and the levels of human GAA (hGAA) in blood and tissues were analyzed by Western blot.

Plasma Collection

Blood samples were collected three months after vector injection by retro-orbital sampling into heparinized capillary tubes, followed by plasma isolation.

Tissue Collection

At the end of the study (3 months post-injection), animals were sacrificed by CO₂ inhalation. Liver, heart, brain, spinal cord, diaphragm, quadriceps, and triceps were collected and snap-frozen in liquid nitrogen for biochemical analysis. Frozen samples were stored at −80° C. until processing.

Western-Blot Analyses

Mouse tissues were collected at sacrifice and homogenized in PBS. Protein concentration was determined using the BCA Protein Assay (Thermo Fisher Scientific, Waltham, Mass.). Plasma and tissues samples were diluted 1:20 in water (except for brain, where samples were diluted 1:200). SDS-page electrophoresis was performed in a 4-12% gradient polyacrylamide gel. After transfer, the membrane was blocked with Odyssey buffer (Li-Cor Biosciences, Lincoln, Nebr.), incubated with an anti-human GAA antibody (rabbit monoclonal antibody, Abcam, Cambridge, UK) and an anti-GAPDH antibody (rabbit polyclonal antibodies, PA1-988, Life Technologies, Carlsbad, Calif.). The membrane was washed and incubated with the appropriate secondary antibody (Li-Cor Biosciences, Lincoln, Nebr.), and visualized by Odyssey imaging system (925-32213, Li-Cor Biosciences, Lincoln, Nebr.). To measure the uptake of GAA in tissues, the 70 KDa band (mature form), visualized by the anti-GAA antibody was quantified and normalized to the levels of expression of GAPDH used as loading control.

Animals presented low GAA levels in plasma before the start of the pharmacological chaperone treatments were removed from the analysis.

Results

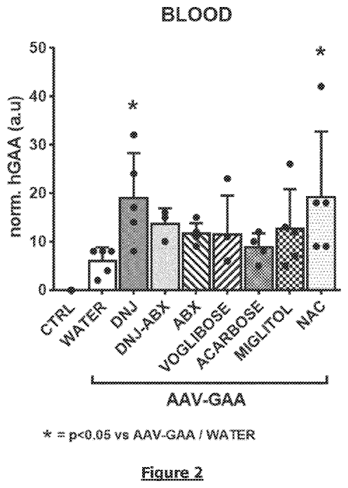

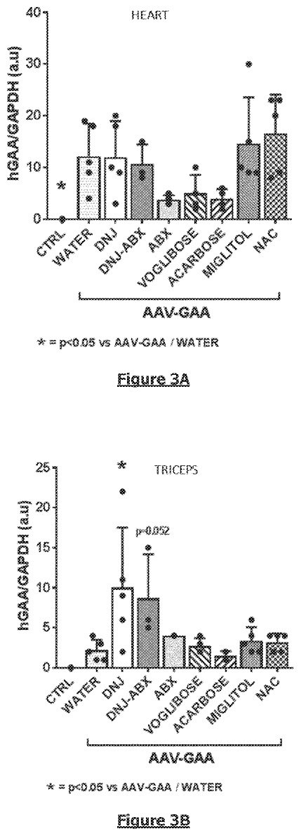

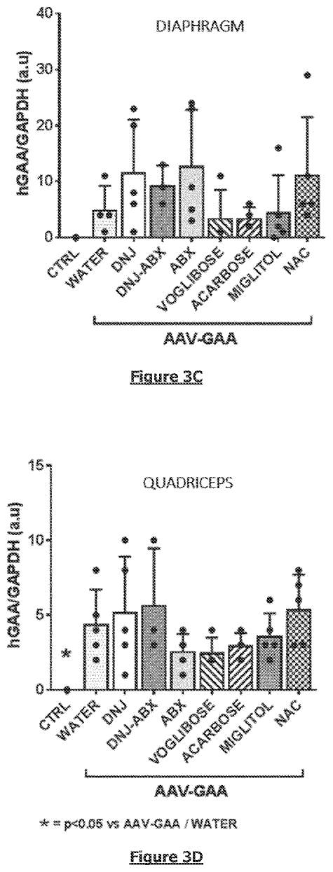

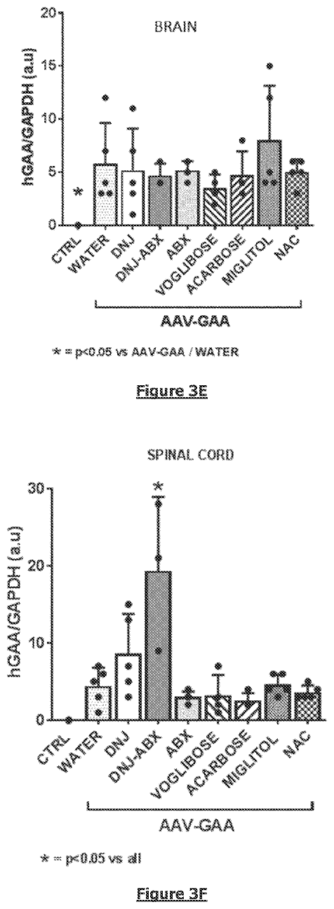

We observed a significant increase in the levels of circulating hGAA in mice treated with DNJ and NAC as measured by Western blot (FIG. 2). In tissues, after uptake, the immature form of hGAA secreted by the liver is taken up and modified by proteolytic cleavage to a 70 KDa form. The 70 KDa form (mature form) is obtained only after proteolytic digestion of the precursor in lysosomes. Therefore, this form is intracellular and its quantification allows estimating the uptake in tissues. The quantification of the mature lysosomal form of hGAA in tissues (by measurement of the band density of the Western Blot) showed increased levels of the enzyme in diaphragm, triceps and spinal cord of mice treated with DNJ and DNJ-ABX although it reached the significance in triceps only for DNJ treated group (FIG. 3).

No differences were observed in heart, quadriceps and brain.

Interestingly the combination of DNJ and ABX led to a significant increase in the levels of hGAA in spinal cord (FIG. 3) compared to levels measured in mice drinking DNJ alone or water. These data support the synergic effect of the combined administration of DNJ and ABX to enhance gene therapy efficacy.

### Example 2: Study on GAA KO (Knock-Out or Deficient) Mice

Materials and Methods

AAV Vector Production

AAV8 vectors were produced as described above in example 1.

In Vivo Studies

Standard animal care and housing were in accordance with the national guidelines. Animal experiments were approved by the ethical committee of the CERFE (Approval Number: 2017-11-B #13643) in accordance with the European Directive 2010/63/EU.

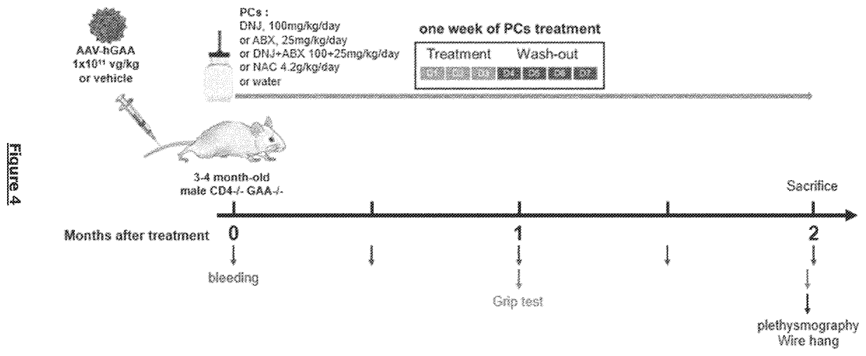

Three to four-month-old GAA deficient male mice were divided into six groups and one control group was composed by wild-type littermates. Each group was composed by eight mice in maximum two cages, to facilitate the administration of the drugs dissolved in drinking water. Starting from day 0, GAA deficient (KO) mice (mouse strain B6; 129-Gaa^{tm1Rabn}/J) were injected with AAV-hGAA at 1×10¹¹ vg/kg via tail vein injection in combination with the different pharmacological chaperones (PC) dissolved in the drinking water. In parallel, untreated GAA wild-type mice and two groups of GAA deficient mice injected with the AAV-hGAA vector or PBS were used as controls. PC molecules were orally administered to the mice, using a “3 days on/4 days off” regimen, consisting in three consecutive days of treatment followed by four consecutive days with drinking water only, as described in FIG. 4. Mice receive 100 mg/kg/die of duvoglustat (DNJ, AX61Q1, Interchim, San Diego, Calif.), 100 mg/kg/die of duvoglustat combined with 25 mg/kg/die of ambroxol hydrochloride (DNJ-ABX), 25 mg/kg/die of ambroxol hydrochloride (ABX, A9797, Sigma, Saint Louis, Mo.), 4200 mg/kg/die of N-acetyl-cysteine (NAC, A7250, Sigma, Saint Louis, Mo.) dissolved in drinking water (FIG. 4) or normal drinking water.

Circulating hGAA activity and levels were monitored over a period of two months. Mice were sacrificed and different organs and muscles were collected (heart, diaphragm, quadriceps, and triceps) to measure the uptake of GAA in tissues and the glycogen clearance.

Plasma Collection

Blood samples were collected by retro-orbital sampling into heparinized capillary tubes, followed by plasma isolation.

Tissue Collection

At the end of the study (2 months post-injection), animals were sacrificed by CO₂ inhalation. Heart, diaphragm, quadriceps, and triceps were collected and snap-frozen in liquid nitrogen for biochemical analysis. Frozen samples are stored at −80° C. until processing.

Measurement of GAA Activity

GAA activity was assessed in plasma and tissue by measurement of 4-methyl-umbelliferyl-α-d-glucoside (4-MU, Sigma, St Louis, Mo.) cleavage at pH 4.3. To this end, mouse tissues collected at sacrifice was homogenized in PBS. Insoluble proteins were removed by centrifugation. The protein content of the resultant lysates was quantified via the BCA Protein Assay (Thermo Fisher Scientific, Waltham, Mass.). Plasma and tissues samples were diluted in water and 10 μL of each sample were incubated with 20 μL of reconstituted substrate for 1 hr at 37° C. After stopping the reaction, the released fluorescence was measured with EN-SPIRE® fluorimeter (Perkin Elmer, Waltham, Mass.). GAA activity was normalized against total protein content or plasma volume.

Western-Blot Analyses

Mouse tissues were collected at sacrifice and homogenized in PBS. Western-Blot analyses were conducted following methods described in Example 1.

Analysis of Glycogen Content

Glycogen content was measured indirectly in tissue homogenates as the glucose released after total digestion with Aspergillus Niger amyloglucosidase (Sigma Aldrich). Samples were incubated for 5 min at 95° C. and then cooled at 4° C. 25 μl of amyloglucosidase diluted 1:50 in 0.1M potassium acetate pH5.5 was then added to each sample. A control reaction without amyloglucosidase was prepared for each sample. Both sample and control reactions were incubated at 37° C. for 90 minutes. The reaction was stopped by incubating samples for 5 min at 95° C. The glucose released was determined using a glucose assay kit (Sigma Aldrich) and by measuring resulting absorbance on an EnSpire alpha plate reader (Perkin-Elmer) at 540 nm.

Results

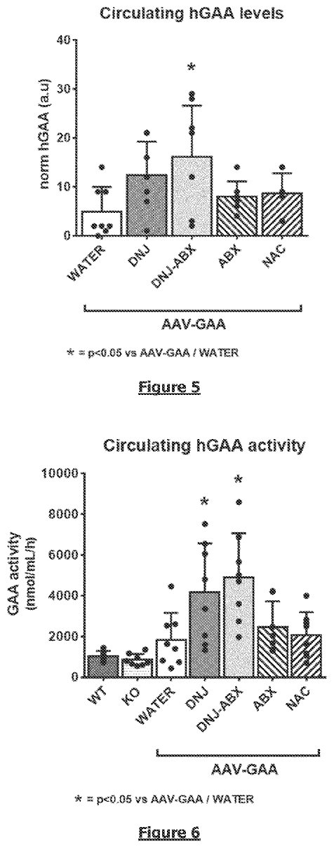

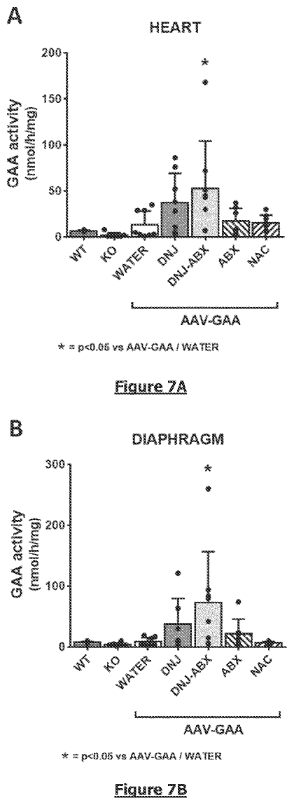

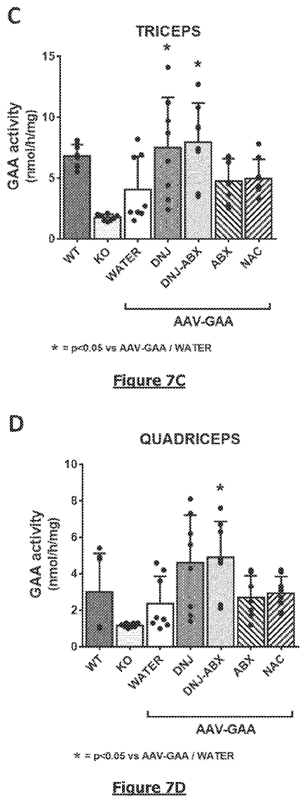

We observed a significant increase in the levels of circulating hGAA in mice treated with DNJ and the combination DNJ-ABX, as measured by Western blot (FIG. 5). These results are in line with those of circulating GAA activity (FIG. 6). In tissues, the GAA activity measurement indicated an increased enzyme activity in heart, diaphragm, triceps and quadriceps of mice treated with DNJ and DNJ-ABX, although it reached the significance only in the group of mice treated with both DNJ and ABX (FIGS. 7A to 7D). No significant differences were observed in mice injected with ABX only or with NAC.

These data obtained in GAA deficient mice, confirm those previously obtained in wild-type mice, and support the synergic effect of the combined administration of DNJ and ABX to enhance gene therapy efficacy.

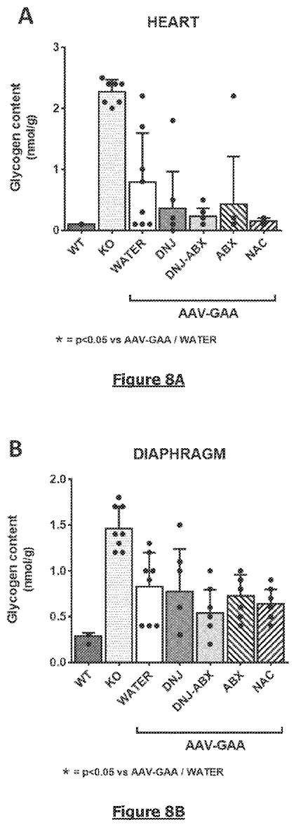

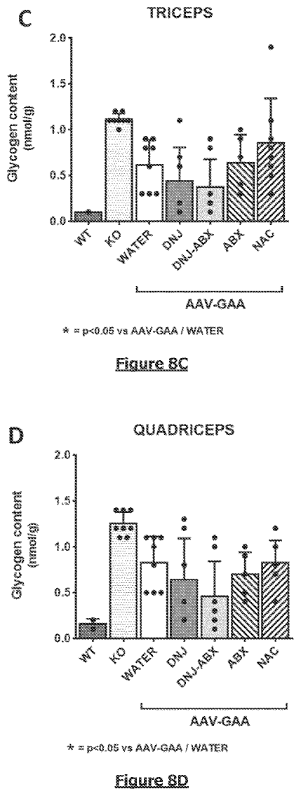

GAA activity resulted in reduced glycogen accumulation in tissue, particularly in heart, where glycogen levels were very similar to those observed to wild-type mice (FIG. 8A). A partial normalization of glycogen content was observed in the other tissues (FIGS. 8B-D). Even if the mice treated by DNJ-ABX presented lower levels of glycogen than mice treated by AAV only, the difference did not reached the significance.

### Example 3: Study of the Effect of Pharmacological Chaperones in Combination with Alglucosidase Alfa (ERT)

Materials and Methods

In Vivo Studies

Standard animal care and housing were in accordance with the national guidelines. Animal experiments were approved by the ethical committee of the CERFE (Approval Number: 2017-11-B #13643) in accordance with the European Directive 2010/63/EU.

Three to four-month-old GAA deficient male mice were divided into three groups and one control group was composed by wild-type littermates.

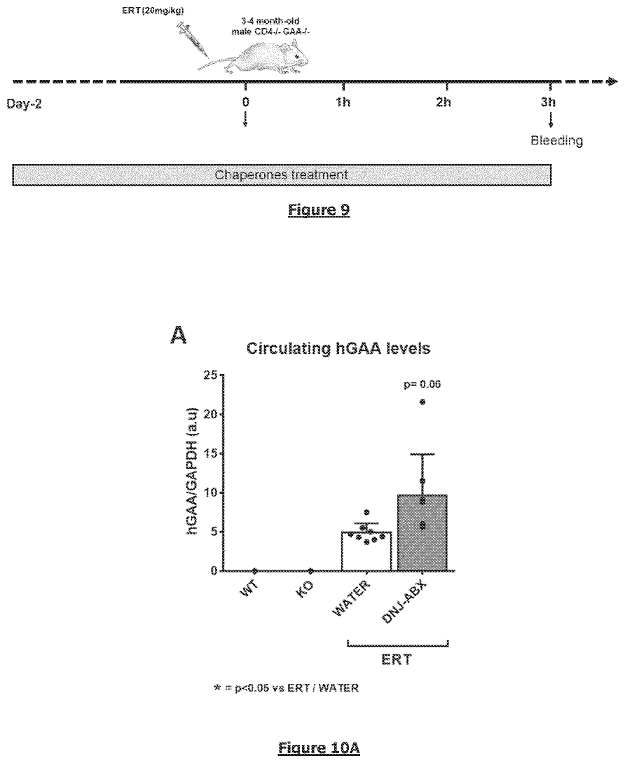

Starting from day −2, one group of GAA deficient (KO) mice (mouse strain B6; 129-Gaa^{tm1Rabn}/J) received pharmacological chaperones (PC) treatment, dissolved in the drinking water, as described in FIG. 9. Mice received 100 mg/kg/die of duvoglustat (DNJ, AX61Q1, Interchim, San Diego, Calif.) combined with 25 mg/kg/die of ambroxol hydrochloride (ABX, A9797, Sigma, Saint Louis, Mo.). In parallel, GAA wild-type mice and two groups of GAA deficient mice were used as controls, receiving normal drinking water. At day 0, GAA deficient mice that were treated with PC and one control group of GAA deficient mice were injected with alglucosidase alpha (ERT, Myozyme, Genzyme, Cambridge, Mass.) at 20 mg/kg via tail vein injection. Circulating GAA activity and levels were monitored three hours after ERT.

Plasma Collection

Blood samples were collected by retro-orbital sampling into heparinized capillary tubes, followed by plasma isolation.

Measurement of GAA Activity

GAA activity was assessed in plasma and tissue by measurement of 4-methyl-umbelliferyl-α-d-glucoside (4-MU, Sigma, St Louis, Mo.) cleavage at pH 4.3. To this end, mouse tissues collected at sacrifice was homogenized in PBS. Insoluble proteins were removed by centrifugation. The protein content of the resultant lysates was quantified via the BCA Protein Assay (Thermo Fisher Scientific, Waltham, Mass.). Plasma and tissues samples were diluted in water and 10 μL of each sample were incubated with 20 μL of reconstituted substrate for 1 hr at 37° C. After stopping the reaction, the released fluorescence was measured with EN-SPIRE® fluorimeter (Perkin Elmer, Waltham, Mass.). GAA activity was normalized against total protein content or plasma volume.

Western-Blot Analyses

Western-Blot analyses were conducted following methods described in Example 1.

Results

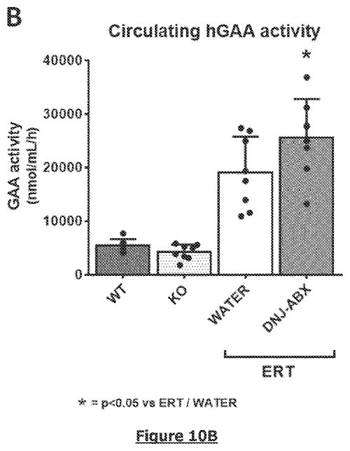

The half-life of recombinant hGAA (ERT) is relatively short. For this reason, the measurement of levels and activity were done at 3 hours post infusion. Compared to mice treated by ERT only, we observed an increase of circulating GAA levels and activity in mice co-treated with the combination DNJ-ABX (FIG. 10).

These data support the synergic effect of the combined administration of DNJ and ABX to enhance GAA bioavailability. Moreover, it shows that the chaperones of the invention are useful for enhancing the treatment efficacy of conventional ERT such as treatment with alglucosidase alpha.

### REFERENCES CITED UNDER THE FORM “[REFERENCE NUMBER]”

1. Hirschhorn, R. and Reuser, A. J. 2001 In The Metabolic and Molecular Basis for Inherited Disease (Scriver, C. R., Beaudet, A. L., Sly, W. S. & Valle, D. Eds.), pp. 3389-3419. McGraw-Hill, New York, see pages 3403-3405.
2. Van der Ploeg and Reuser, 2008 Lancet 372: 1342-1351,
3. Van den Hout et al., 2003, Pediatrics; 112:332-340 [PubMed: 12897283]
4. DeRuisseau et al., 2009; Proc Natl Acad Sci USA, 106:9419-9424. [PubMed:19474295]
5. Amalfitano, A. et al., 2001 Genet. In Med. 3:132-138
6. Kikuchi et al., 1998, Clinical and metabolic correction of Pompe disease by enzyme therapy in acid maltase-deficient quail. The Journal of Clinical Investigation; 101:827-833. [PubMed: 9466978]
7 Raben et al., 2003, Molecular Genetics and Metabolism; 80:159-169. [PubMed: 14567965]
8. Xu et al., JCI Insight 2019; 4(5):e125358
9. Lukas et al, The American society of Gene and Cell Therapy 2015, vol. 23 no. 3, 456-464)
10. Hoefsloot et al., 1988 EMBO J. 7: 1697;
11. Martiniuk et al., 1990 DNA and Cell Biology 9: 85)
12. Hoefsloot et al., 1990 Biochem. J. 272: 485;
13. Wisselaar et al., 1993 J. Biol. Chem. 268: 2223;
14. Hermans et al., 1993 Biochem. J. 289: 681).
15. Van Hove et al., 1996 Proc. Natl. Acad. Sci. USA 93: 65 (human)
16. Kunita et al., 1997 Biochemica et Biophysica Acta 1362: 269;
17 Ill, C. R., et al. (1997). Optimization of the human factor VIII complementary DNA expression plasmid for gene therapy of hemophilia A. Blood Coag. Fibrinol. 8: S23-S30
18. Rincon et al., Mol Ther. 2015 January; 23(1):43-52,
19. Chuah et al., Mol Ther. 2014 September; 22(9):1605-13
20. Nair et al., Blood. 2014 May 15; 123(20):3195-9.
21. Mates et al. 2009 Nat Genet. 2009 June; 41(6):753-61. doi: 10.1038/ng.343
22. Ling et al., 2016 Jul. 18, Hum Gene Ther Methods.
23. Vercauteren et al., 2016, Mol. Ther. Vol. 24(6), p. 1042
24. Rosario et al., 2016, Mol Ther Methods Clin Dev. 3, p. 16026
25. Shen et al., 2007 Molecular Therapy, volume 15, issue 11, pages 1955-1962
26. Tenney et al., 2014, Virology, volumes 454-455, April 2014, pages 227-236
27. Bartel et al., 2011, Front. Microbiol., 4 Oct. 2011 https://doi.org/10.3389/fmicb.2011.00204
28. Michelfelder et al. (PLoS ONE, 2009, 4, e5122
29. Zhong et al., 2008 PNAS Jun. 3, 2008 105 (22) 7827-7832;
https://doi.org/10.1073/pnas.0802866105
30. Finet et al., Virology, 2018, 513, 43-51).
31. McCarty et al., 2003 Gene Therapy December; 10(26):2112-8.
32. Matsushita et al., Gene Therapy, 1998, 5, 938-945
33. Ayuso et al., Gene Therapy, 2010, 17, 503-510
34. Zhang et al, Hum Gen Ther, 2012, Volume 23, number 5:460-472.

Sequence listing

| SEQ ID NO: | NAME |
|---|---|
| SEQ ID NO: 1 | hGAAwt w:o sp 925 (PRT) |
| SEQ ID NO: 2 | PRT 952 homosapiens (PRT) |
| SEQ ID NO: 3 | Sp7 (PRT) |
| SEQ ID NO: 4 | Sp1 (PRT) |
| SEQ ID NO: 5 | Sp2 (PRT) |
| SEQ ID NO: 6 | Sp6 (PRT) |
| SEQ ID NO: 7 | Sp8 (PRT) |
| SEQ ID NO: 8 | Homo sapiens hGAA (nt) |
| SEQ ID NO: 9 | hGAA wt w/o sp (nt) |
| SEQ ID NO: 10 | hGAA co1 w/o sp (nt) |
| SEQ ID NO: 11 | hGAA co2 w/o sp (nt) |
| SEQ ID NO: 12 | delta 8 co1 GAA (nt) |
| SEQ ID NO: 13 | delta 8 co2 GAA (nt) |
| SEQ ID NO: 14 | hAAT promoter (nt) |
| SEQ ID NO: 15 | ApoE control region (nt) |
| SEQ ID NO: 16 | HBB2 (nt) |
| SEQ ID NO: 17 | modified HBB2 (nt) |
| SEQ ID NO: 18 | FIX intron (nt) |
| SEQ ID NO: 19 | Modified FIX intron (nt) |
| SEQ ID NO: 20 | chicken beta globin intron (nt) |
| SEQ ID NO: 21 | Modified chicken beta globin intron (nt) |
| SEQ ID NO: 22 | Sp2 + hGAAwt-delta8 (nt) |
| SEQ ID NO: 23 | Sp2 + hGAAcol-delta8 (nt) |
| SEQ ID NO: 24 | Sp2 + hGAAco2-delta8 (nt) |
| SEQ ID NO: 25 | sp7 + hGAA co1-delta 8 (nt) |
| SEQ ID NO: 26 | Sp7 + hGAA co1-delta 42 (nt) |
| SEQ ID NO: 27 | hGAA-delta-8 (PRT) |
| SEQ ID NO: 28 | hGAA-delta-42 (PRT) |
| SEQ ID NO: 29 | GAA 952aa homosapiens (PRT) |
| SEQ ID NO: 30 | GAA 952aa homosapiens (PRT) |
| SEQ ID NO: 31 | GAA 952aa homosapiens (PRT) |
| SEQ ID NO: 32 | GAA 952aa homosapiens (PRT) |
| SEQ ID NO: 33 | variant hGAAwt w/o sp 925 aa (PRT) |
| SEQ ID NO: 34 | hGAA-delta-29 (PRT) |
| SEQ ID NO: 35 | hGAA-delta-43 (PRT) |
| SEQ ID NO: 36 | hGAA-delta-47 (PRT) |
| SEQ ID NO: 37 | Sp7 + hGAAwt-delta 29 (nt) |
| SEQ ID NO: 38 | Sp7 + hGAAco1-delta 29 (nt) |
| SEQ ID NO: 39 | Sp7 + hGAAco2-delta 29 (nt) |
| SEQ ID NO: 40 | Sp7 + hGAAwt-delta 42 (nt) |
| SEQ ID NO: 41 | Sp7 + hGAAco2-delta 42 (nt) |
| SEQ ID NO: 42 | Sp7 + hGAAwt-delta 43 (nt) |
| SEQ ID NO: 43 | Sp7 + hGAAco1-delta 43 (nt) |
| SEQ ID NO: 44 | Sp7 + hGAAco2-delta 43 (nt) |
| SEQ ID NO: 45 | Sp7 + hGAAwt-delta 47 (nt) |
| SEQ ID NO: 46 | Sp7 + hGAAco1-delta 47 (nt) |
| SEQ ID NO: 47 | Sp7 + hGAAco2-delta 47 (nt) |
| SEQ ID NO: 48 | hGAAco1 delta 42 w/o sp (nt) |
| SEQ ID NO: 49 | hGAAco2 delta 42 w/o sp (nt) |
| SEQ ID NO: 50 | hGAAco1 delta 43 w/o sp (nt) |
| SEQ ID NO: 51 | hGAAco2 delta 43 w/o sp (nt) |
| SEQ ID NO: 52 | hGAAco1 delta 47 w/o sp (nt) |
| SEQ ID NO: 53 | hGAAco2 delta 47 w/o sp (nt) |
| SEQ ID NO: 54 | Sp7 (nt) |
| SEQ ID NO: 55 | Promoter LIMP (nt) |

1. A kit of parts comprising (i) pharmacological chaperones or a pharmaceutically acceptable salt thereof and (ii) a therapeutic acid-alpha glucosidase (GAA) polypeptide or a nucleic acid molecule encoding a therapeutic GAA polypeptide,
wherein said pharmacological chaperones are 1-deoxynojirimycin (DNJ) or a derivative thereof and ambroxol (ABX) or a derivative thereof.

2. A method of treating a glycogen storage disease (GSD) in a subject in need thereof, the method comprising administering the kit of parts according to claim 1 to the subject in need thereof.

3. A method of increasing the uptake of GAA in a tissue of the nervous system in a subject in need thereof, the method comprising administering the kit of parts according to claim 1 to the subject in need thereof.

4. A method for treating central nervous system (CNS) dysfunctions in GSD in a subject in need thereof, the method comprising administering the kit of parts according to claim 1 to the subject in need thereof.

5. The method according to claim 2, wherein the kit of parts comprises (i) duvoglustat and ambroxol hydrochloride and (ii) a viral vector in which a nucleic acid construct for expressing the nucleic acid molecule encoding a therapeutic GAA polypeptide is inserted.

6. A method of treating a GSD in a subject receiving a therapeutic GAA treatment for treating said GSD, the method comprising administering a composition comprising pharmacological chaperones or a pharmaceutically acceptable salt thereof, wherein said pharmacological chaperones are 1-deoxynojirimycin (DNJ) or a derivative thereof and ambroxol (ABX) or a derivative thereof.

7. A method of increasing the uptake of GAA in a tissue of the nervous system in a subject receiving a therapeutic GAA treatment for treating a GSD, the method comprising administering a composition comprising pharmacological chaperones or a pharmaceutically acceptable salt thereof, wherein said pharmacological chaperones are DNJ or a derivative thereof and ABX or a derivative thereof.

8. A method for treating CNS dysfunctions in GSD in a subject receiving a therapeutic GAA treatment for treating said GSD, the method comprising administering a composition comprising pharmacological chaperones or a pharmaceutically acceptable salt thereof, wherein said pharmacological chaperones are DNJ or a derivative thereof and ABX or a derivative thereof.

9. The method according to claim 6, wherein the therapeutic GAA treatment is a nucleic acid molecule encoding a therapeutic GAA polypeptide or a viral vector in which a nucleic acid construct for expressing said nucleic acid molecule is inserted.

10. The method according to claim 6, wherein the therapeutic GAA treatment is a viral vector in which a nucleic acid construct for expressing said nucleic acid molecule is inserted, wherein the pharmacological chaperones are duvoglustat and ambroxol hydrochloride.

11. The kit of parts according to claim 1, wherein:
the DNJ derivative is selected from N-methyl-DNJ, N-butyl-DNJ, N-cyclopropylmethyl-DNJ, N-(2-(N,N-dimethylamido)ethyloxy-DNJ, N-4-t-butyloxycarbonyl-piperidnylmethyl-DNJ, N-2-R-tetrahydrofuranylmethyl-DNJ, N-2-R-tetrahydrofuranylmethyl-DNJ, N-(2-(2,2,2-trifluoroethoxy)ethyl-DNJ, N-2-methoxyethyl-DNJ, N-2-ethoxyethyl-DNJ, N-4-trifluoromethylbenzyl-DNJ, N-alpha-cyano-4-trifluoromethylbenzyl-DNJ, N-4-trifluoromethoxybenzyl-DNJ, N-4-n-pentoxybenzyl-DNJ, N-4-n-butoxybenzyl-DNJ or Cl-nonyl DNJ,
the DNJ is duvoglustat, duvoglustat hydrochloride, miglustat or miglustat hydrochloride, and/or
ABX is ambroxol hydrochloride.

12. The method according to claim 2, wherein the GSD is selected from GSDI, GSDII, GSDIII, GSDIV, GSDV, GSDVI, GSDVII, GSDVIII or lethal congenital glycogen storage disease of the heart.

13. The method of claim 2, wherein the pharmacological chaperones are administered before, simultaneously and/or after the nucleic acid molecule encoding a therapeutic GAA polypeptide.

14. The kit of parts of claim 1, wherein the nucleic acid molecule encoding a therapeutic GAA polypeptide is inserted into a nucleic acid construct for expressing said nucleic acid molecule, said nucleic acid construct being inserted into a viral vector selected from a retroviral vector or an AAV vector.

15. The kit of parts of claim 1, wherein the therapeutic GAA polypeptide comprises a GAA polypeptide moiety and a signal peptide moiety fused to the N-terminal of the GAA polypeptide moiety, wherein said signal peptide moiety fused to the N-terminal of the GAA polypeptide moiety is selected from SEQ ID NO: 3, SEQ ID NO: 4, SEQ ID NO: 5, SEQ ID NO: 6 or SEQ ID NO: 7, and said GAA polypeptide moiety is selected from SEQ ID NO: 27, SEQ ID NO: 28, SEQ ID NO: 34, SEQ ID NO: 35 or SEQ ID NO: 36.

16. The kit of parts of claim 1, wherein said nucleic acid molecule is selected from SEQ ID NO: 8, SEQ ID NO: 22, SEQ ID NO: 23, SEQ ID NO: 24, SEQ ID NO: 25, SEQ ID NO: 26, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 or SEQ ID NO: 47.

17. The method of claim 7, wherein the therapeutic GAA treatment is a nucleic acid molecule encoding a therapeutic GAA polypeptide.

18. The method of claim 17, wherein the pharmacological chaperones are administered before, simultaneously and/or after the nucleic acid molecule encoding a therapeutic GAA polypeptide.

19. The method of claim 7, wherein the therapeutic GAA treatment is a viral vector in which a nucleic acid construct for expressing a nucleic acid molecule encoding a therapeutic GAA polypeptide is inserted, wherein the pharmacological chaperones are duvoglustat and ambroxol hydrochloride.

20. The method of claim 7, wherein the method increases the uptake of GAA in spinal cord.
